SEQUENCE LISTING (20 sequences)

SEQ ID NO: 1 - protein, 388 aa, artificial
  protein sequence of human Fc-MDL-1 fusion protein (NH-Fc-MDL-1-COOH)
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKPQIFNKSNDGFTTTRSYGTVSQIFGSSSPSPNGFITTRSYGTVCPKDWEFYQARCFFLSTSESSWNESRDFCKGKGSTLAIVNTPEKLKFLQDITDAEKYFIGLIYHREEKRWRWINNSVFNGNVTNQNQNFNCATIGLTKTFDAASCDISYRRICEKNAK

SEQ ID NO: 2 - DNA, 1167 bp, artificial
  nucleotide sequence of human Fc-MDL-1 fusion protein (5′-Fc-MDL-1-3′)
gacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaccacagatttttaacaaaagtaacgatggtttcaccaccaccaggagctatggaacagtctcacagatttttgggagcagttccccaagtcccaacggcttcattaccacaaggagctatggaacagtctgccccaaagactgggaattttatcaagcaagatgttttttcttatccacttctgaatcatcttggaatgaaagcagggacttttgcaaaggaaaaggatccacattggcaattgtcaacacgccagagaaactgaagtttcttcaggacataactgatgctgagaagtattttattggcttaatttaccatcgtgaagagaaaaggtggcgttggatcaacaactctgtgttcaatggcaatgttaccaatcagaatcagaatttcaactgtgcgaccattggcctaacaaagacatttgatgctgcatcatgtgacatcagctaccgcaggatctgtgagaagaatgccaaatga

SEQ ID NO: 3 - protein, 388 aa, artificial
  protein sequence of human Fc-MDL-1 fusion protein (NH-MDL-1-Fc-COOH)
PQIFNKSNDGFTTTRSYGTVSQIFGSSSPSPNGFITTRSYGTVCPKDWEFYQARCFFLSTSESSWNESRDFCKGKGSTLAIVNTPEKLKFLQDITDAEKYFIGLIYHREEKRWRWINNSVFNGNVTNQNQNFNCATIGLTKTFDAASCDISYRRICEKNAKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - DNA, 1167 bp, artificial
  nucleotide sequence of human Fc-MDL-1 fusion protein (5′-MDL-1-Fc-3′)
ccacagatttttaacaaaagtaacgatggtttcaccaccaccaggagctatggaacagtctcacagatttttgggagcagttccccaagtcccaacggcttcattaccacaaggagctatggaacagtctgccccaaagactgggaattttatcaagcaagatgttttttcttatccacttctgaatcatcttggaatgaaagcagggacttttgcaaaggaaaaggatccacattggcaattgtcaacacgccagagaaactgaagtttcttcaggacataactgatgctgagaagtattttattggcttaatttaccatcgtgaagagaaaaggtggcgttggatcaacaactctgtgttcaatggcaatgttaccaatcagaatcagaatttcaactgtgcgaccattggcctaacaaagacatttgatgctgcatcatgtgacatcagctaccgcaggatctgtgagaagaatgccaaagacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 5 - protein, 392 aa, artificial sequence
  protein sequence of mouse Fc-MDL-1 fusion protein (NH-Fc-MDL-1-COOH)
VPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFRCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGKASPQVFGKSNDGFVPTESYGTTSVQNVSQIFGRNDESTMPTRSYGTVCPRNWDFHQGKCFFFSFSESPWKDSMDYCATQGSTLAIVNTPEKLKYLQDIAGIENYFIGLVRQPGEKKWRWINNSVFNGNVTNQDQNFDCVTIGLTKTYDAASCEVSYRWICEMNAK

SEQ ID NO: 6 - DNA, 1179 bp, artificial
  nucleotide sequence of mouse Fc-MDL-1 fusion protein (5′-Fc-MDL-1-3′)
gtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcagatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaagctagcccacaggtttttggcaaaagtaatgatggcttcgtccccacggagagctacggaaccactagtgtgcagaatgtctcacagatctttgggagaaatgacgaaagtaccatgcctacaaggagctatggaacagtctgtcccagaaactgggattttcaccaaggaaaatgctttttcttctccttctccgaatcaccttggaaagacagcatggattattgtgcaacacaaggatccacactggcaattgtcaacactccagagaaactgaagtatcttcaggacatagctggtattgagaattactttattggtttggtacgtcagcctggagagaaaaagtggcgctggatcaacaactctgtgttcaatggcaatgttaccaatcaggaccagaacttcgactgtgtcactataggtctgacgaagacatatgatgctgcatcatgtgaagtcagctatcgctggatctgcgaaatgaatgccaaatga

SEQ ID NO: 7 - protein, 227 aa, artificial
  protein sequence of human Fc portion
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - DNA, 681 bp, artificial
  nucleotide sequence of human Fc portion
gacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 9 - protein, 227 aa, artificial
  protein sequence of mouse Fc
VPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFRCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 10 - DNA, 681 bp, artificial
  nucleotide sequence of mouse Fc
gtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcagatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaa

SEQ ID NO: 11 - protein, 161 aa, artificial
  protein sequence of extracellular domain of human MDL-1
PQIFNKSNDGFTTTRSYGTVSQIFGSSSPSPNGFITTRSYGTVCPKDWEFYQARCFFLSTSESSWNESRDFCKGKGSTLAIVNTPEKLKFLQDITDAEKYFIGLIYHREEKRWRWINNSVFNGNVTNQNQNFNCATIGLTKTFDAASCDISYRRICEKNAK

SEQ ID NO: 12 - DNA, 486 bp, artificial
  nucleotide sequence of extracellular domain of human MDL-1
ccacagatttttaacaaaagtaacgatggtttcaccaccaccaggagctatggaacagtctcacagatttttgggagcagttccccaagtcccaacggcttcattaccacaaggagctatggaacagtctgccccaaagactgggaattttatcaagcaagatgttttttcttatccacttctgaatcatcttggaatgaaagcagggacttttgcaaaggaaaaggatccacattggcaattgtcaacacgccagagaaactgaagtttcttcaggacataactgatgctgagaagtattttattggcttaatttaccatcgtgaagagaaaaggtggcgttggatcaacaactctgtgttcaatggcaatgttaccaatcagaatcagaatttcaactgtgcgaccattggcctaacaaagacatttgatgctgcatcatgtgacatcagctaccgcaggatctgtgagaagaatgccaaatga

SEQ ID NO: 13 - protein, 163 aa, artificial
  protein sequence of extracellular domain of mouse MDL-1
PQVFGKSNDGFVPTESYGTTSVQNVSQIFGRNDESTMPTRSYGTVCPRNWDFHQGKCFFFSFSESPWKDSMDYCATQGSTLAIVNTPEKLKYLQDIAGIENYFIGLVRQPGEKKWRWINNSVFNGNVTNQDQNFDCVTIGLTKTYDAASCEVSYRWICEMNAK

SEQ ID NO: 14 - DNA, 492 bp, artificial
  nucleotide sequence of extracellular domain of mouse MDL-1
ccacaggtttttggcaaaagtaatgatggcttcgtccccacggagagctacggaaccactagtgtgcagaatgtctcacagatctttgggagaaatgacgaaagtaccatgcctacaaggagctatggaacagtctgtcccagaaactgggattttcaccaaggaaaatgctttttcttctccttctccgaatcaccttggaaagacagcatggattattgtgcaacacaaggatccacactggcaattgtcaacactccagagaaactgaagtatcttcaggacatagctggtattgagaattactttattggtttggtacgtcagcctggagagaaaaagtggcgctggatcaacaactctgtgttcaatggcaatgttaccaatcaggaccagaacttcgactgtgtcactataggtctgacgaagacatatgatgctgcatcatgtgaagtcagctatcgctggatctgcgaaatgaatgccaaatga

SEQ ID NO: 15 - protein, 188 aa, human
MNWHMIISGLIVVVLKVVGMTLFLLYFPQIFNKSNDGFTTTRSYGTVSQIFGSSSPSPNGFITTRSYGTVCPKDWEFYQARCFFLSTSESSWNESRDFCKGKGSTLAIVNTPEKLKFLQDITDAEKYFIGLIYHREEKRWRWINNSVFNGNVTNQNQNFNCATIGLTKTFDAASCDISYRRICEKNAK

SEQ ID NO: 16 - DNA, 570 bp, human
catgaactggcacatgatcatctctgggcttattgtggtagtgcttaaagttgttggaatgaccttatttctactttatttcccacagatttttaacaaaagtaacgatggtttcaccaccaccaggagctatggaacagtctcacagatttttgggagcagttccccaagtcccaacggcttcattaccacaaggagctatggaacagtctgccccaaagactgggaattttatcaagcaagatgttttttcttatccacttctgaatcatcttggaatgaaagcagggacttttgcaaaggaaaaggatccacattggcaattgtcaacacgccagagaaactgaagtttcttcaggacataactgatgctgagaagtattttattggcttaatttaccatcgtgaagagaaaaggtggcgttggatcaacaactctgtgttcaatggcaatgttaccaatcagaatcagaatttcaactgtgcgaccattggcctaacaaagacatttgatgctgcatcatgtgacatcagctaccgcaggatctgtgagaagaatgccaaatgatc

SEQ ID NO: 17 - protein, 190 aa, muscus
MNWHMIISGLIVVVIKVVGMTFFLLYFPQVFGKSNDGFVPTESYGTTSVQNVSQIFGRNDESTMPTRSYGTVCPRNWDFHQGKCFFFSFSESPWKDSMDYCATQGSTLAIVNTPEKLKYLQDIAGIENYFIGLVRQPGEKKWRWINNSVFNGNVTNQDQNFDCVTIGLTKTYDAASCEVSYRWICEMNAK

SEQ ID NO: 18 - DNA, 590 bp, muscus
cccatcatcatgaactggcacatgatcatctcggggcttatcgtagtagtgatcaaagttgttggaatgaccttttttctgctgtatttcccacaggtttttggcaaaagtaatgatggcttcgtccccacggagagctacggaaccactagtgtgcagaatgtctcacagatctttgggagaaatgacgaaagtaccatgcctacaaggagctatggaacagtctgtcccagaaactgggattttcaccaaggaaaatgctttttcttctccttctccgaatcaccttggaaagacagcatggattattgtgcaacacaaggatccacactggcaattgtcaacactccagagaaactgaagtatcttcaggacatagctggtattgagaattactttattggtttggtacgtcagcctggagagaaaaagtggcgctggatcaacaactctgtgttcaatggcaatgttaccaatcaggaccagaacttcgactgtgtcactataggtctgacgaagacatatgatgctgcatcatgtgaagtcagctatcgctggatctgcgaaatgaatgccaaatgatcatagat

SEQ ID NO: 19 - DNA, 9276 bp, artificial
  Expression vector pPEP1-hFc-MDL-1, encoding human Fc-MDL-1 fusionprotein
ggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatctctagtcaaggcactatacatcaaatattccttattaacccctttacaaattaaaaagctaaaggtacacaatttttgagcatagttattaatagcagacactctatgcctgtgtggagtaagaaaaaacagtatgttatgattataactgttatgcctacttataaaggttacagaatatttttccataattttcttgtatagcagtgcagctttttcctttgtggtgtaaatagcaaagcaagcaagagttctattactaaacacagcatgactcaaaaaacttagcaattctgaaggaaagtccttggggtcttctacctttctcttcttttttggaggagtagaatgttgagagtcagcagtagcctcatcatcactagatggcatttcttctgagcaaaacaggttttcctcattaaaggcattccaccactgctcccattcatcagttccataggttggaatctaaaatacacaaacaattagaatcagtagtttaacacattatacacttaaaaattttatatttaccttagagctttaaatctctgtaggtagtttgtccaattatgtcacaccacagaagtaaggttccttcacaaagatctaaagccagcaaaagtcccatggtcttataaaaatgcatagctttaggaggggagcagagaacttgaaagcatcttcctgttagtctttcttctcgtagacttcaaacttatacttgatgcctttttcctcctggacctcagagaggacgcctgggtattctgggagaagtttatatttccccaaatcaatttctgggaaaaacgtgtcactttcaaattcctgcatgatccttgtcacaaagagtctgaggtggcctggttgattcatggcttcctggtaaacagaactgcctccgactatccaaaccatgtctactttacttgccaattccggttgttcaataagtcttaaggcatcatccaaacttttggcaagaaaatgagctcctcgtggtggttctttgagttctctactgagaactatattaattctgtcctttaaaggtcgattcttctcaggaatggagaaccaggttttcctacccataatcaccagattctgtttaccttccactgaagaggttgtggtcattctttggaagtacttgaactcgttcctgagcggaggccagggtaggtctccgttcttgccaatccccatattttgggacacggcgacgatgcagttcaatggtcgaaccatgatggcagcggggataaaatcctaccagccttcacgctaggattgccgtcaagtttggcgcgaaatcgcagccctgagctgtccccccccccaagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgaagcctatagagtacgagccatagatagaataaaagattttatttagtctccagaaaaaggggggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtaccgagctcgaattgatctcgaggaactgaaaaaccagaaagttaactggtaagtttagtctttttgtcttttatttcaggtcccggatcggaattcccatcgccgcggcatcgatcgggcccgagatctcgcgcgcctgggtaacatgcttggggtcctggtccttggcgcgctggccctggcaggcctggtcttccccgaggcagacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaccacagatttttaacaaaagtaacgatggtttcaccaccaccaggagctatggaacagtctcacagatttttgggagcagttccccaagtcccaacggcttcattaccacaaggagctatggaacagtctgccccaaagactgggaattttatcaagcaagatgttttttcttatccacttctgaatcatcttggaatgaaagcagggacttttgcaaaggaaaaggatccacattggcaattgtcaacacgccagagaaactgaagtttcttcaggacataactgatgctgagaagtattttattggcttaatttaccatcgtgaagagaaaaggtggcgttggatcaacaactctgtgttcaatggcaatgttaccaatcagaatcagaatttcaactgtgcgaccattggcctaacaaagacatttgatgctgcatcatgtgacatcagctaccgcaggatctgtgagaagaatgccaaatgagcggccgctgcagatcgatgccgcggcgatatcgctcgaggaagcttcgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatccggctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtattcgagctcggacattgattattgactagtggaccccgtccctaacccacggggcccgtggctatggcagggcttgccgccccgacgttggctgcgagccctgggccttcacccgaacttggggggtggggtggggaaaaggaagaaacgcgggcgtattggccccaatggggtctcggtggggtatcgacagagtgccagccctgggaccgaaccccgcgtttatgaacaaacgacccaacaccgtgcgttttattctgtctttttattgccgtcatagcgcgggttccttccggtattgtctccttccgtgtttcagttagcctcccccatctccccagatctgcacccaatcggcaggcacgggcggcgatctccaatctgcgggatcagtcagatcacccgagtgcgtgggcatgacaatcgtgccctggggaccaacacaatccagaagggcctgaatcactgcgaccggccctcccgcgacccagccgagcgagcttagcgaactgtggacgagaactgtgccaccaagcgtaaggccgttctctcgcatttgccttgctaggctcgcgcgagttgctggctgaggcgttctcgaaatcagctcttgttcggtcggcatctactctattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatggcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatatctcccggatctgcggcacgctgttgacgctgttaagcgggtcgctgcagggtcgctcggtgttcgaggccacacgcgtcaccttaatatgcgaagtggacctgggaccgcgccgccccgactgcatctgcgtgttcgaattcgccaatgacaagacgctgggcggggtttgtgtcatcatagaactaaagacatgcaaatatatttcttccggggacaccgccagcaaacgcgagcaacgggccacggggatgaagcagttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatcaacaaaaagcaggatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattctcatgtttgacagcttatcatcgat

SEQ ID NO: 20 - DNA, 11025 bp, artificial
  Expression Vector pPEP1-mFc-MDL-1, encoding mouse Fc-MDL-1 fusionprotein
tcaataagtcttaaggcatcatccaaacttttggcaagaaaatgagctcctcgtggtggttctttgagttctctactgagaactatattaattctgtcctttaaaggtcgattcttctcaggaatggagaaccaggttttcctacccataatcaccagattctgtttaccttccactgaagaggttgtggtcattctttggaagtacttgaactcgttcctgagcggaggccagggtaggtctccgttcttgccaatccccatattttgggacacggcgacgatgcagttcaatggtcgaaccatgatggcagcggggataaaatcctaccagccttcacgctaggattgccgtcaagtttggcgcgaaatcgcagccctgagctgtccccccccccaagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgaagcctatagagtacgagccatagatagaataaaagattttatttagtctccagaaaaaggggggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagatggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactcccctattggtgacgatactttccattactaatccataacatggctctttgccacaactatctctattggctatatgccaatactctgtccttcagagactgacacggactctgtatttttacaggatggggtcccatttattatttacaaattcacatatacaacaacgccgtcccccgtgcccgcagtttttattaaacatagcgtgggatctccacgcgaatctcgggtacgtgttccggacatgggctcttctccggtagcggcggagcttccacatccgagccctggtcccatgcctccagcggctcatggtcgctcggcagctccttgctcctaacagtggaggccagacttaggcacagcacaatgcccaccaccaccagtgtgccgcacaaggccgtggcggtagggtatgtgtctgaaaatgagcgtggagattgggctcgcacggctgacgcagatggaagacttaaggcagcggcagaagaagatgcaggcagctgagttgttgtattctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttcgccaccatgcttggggtcctggtccttggcgcgctggccctggcaggcctggtcttccccgaggtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcagatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaagctagcccacaggtttttggcaaaagtaatgatggcttcgtccccacggagagctacggaaccactagtgtgcagaatgtctcacagatctttgggagaaatgacgaaagtaccatgcctacaaggagctatggaacagtctgtcccagaaactgggattttcaccaaggaaaatgctttttcttctccttctccgaatcaccttggaaagacagcatggattattgtgcaacacaaggatccacactggcaattgtcaacactccagagaaactgaagtatcttcaggacatagctggtattgagaattactttattggtttggtacgtcagcctggagagaaaaagtggcgctggatcaacaactctgtgttcaatggcaatgttaccaatcaggaccagaacttcgactgtgtcactataggtctgacgaagacatatgatgctgcatcatgtgaagtcagctatcgctggatctgcgaaatgaatgccaaatgagcggccgctgcagatcgatgccgcggcgatatcgctcgaggaagcttcgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatccggctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtattcgagctcggacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccacccagcggaccccgtccctaacccacggggccagtggctatggcagggcctgccgccccgacgttggctgcgagccctgggccttcacccgaacttggggggtggggtggggaaaaggaagaaacgcgggcgtattggccccaatggggtctcggtggggtatcgacagagtgccagccctgggaccgaaccccgcgtttatgaacaaacgacccaacacccgtgcgttttattctgtctttttattgccgtcatagcgcgggttccttccggtattgtctccttccgtgtttcagttagcctcccccatctccccagatctgcacccaatcggcaggcacgggcggcgatctccaatctgcgggatcagtcagatcacccgagtgcgtgggcatgacaatcgtgccctggggaccaacacaatccagaagggcctgaatcactgcgaccggccctcccgcgacccagccgagcgagcttagcgaactgtggacgagaactgtgccaccaagcgtaaggccgttctctcgcatttgccttgctaggctcgcgcgagttgctggctgaggcgttctcgaaatcagctcttgttcggtcggcatctactctattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatggcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatatctcccggatctgcggcacgctgttgacgctgttaagcgggtcgctgcagggtcgctcggtgttcgaggccacacgcgtcaccttaatatgcgaagtggacctcggaccgcgccgccccgactgcatctgcgtgttcgaattcgccaatgacaagacgctgggcggggtttgtgtcatcatagaactaaagacatgcaaatatatttcttccggggacaccgccagcaaacgcgagcaacgggccacggggatgaagcagttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggcccgtggccgggggactgttgggcgccatctccttgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcgaattcaggccatgtttgacagcttatcatcgatggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatctctagtcaaggcactatacatcaaatattccttattaacccctttacaaattaaaaagctaaaggtacacaatttttgagcatagttattaatagcagacactctatgcctgtgtggagtaagaaaaaacagtatgttatgattataactgttatgcctacttataaaggttacagaatatttttccataattttcttgtatagcagtgcagctttttcctttgtggtgtaaatagcaaagcaagcaagagttctattactaaacacagcatgactcaaaaaacttagcaattctgaaggaaagtccttggggtcttctacctttctcttcttttttggaggagtagaatgttgagagtcagcagtagcctcatcatcactagatggcatttcttctgagcaaaacaggttttcctcattaaaggcattccaccactgctcccattcatcagttccataggttggaatctaaaatacacaaacaattagaatcagtagtttaacacattatacacttaaaaattttatatttaccttatagctttaaatctctgtaggtagtttgtccaattatgtcacaccacagaagtaaggttccttcacaaagatctaaagccagcaaaagtcccatggtcttataaaaatgcatagctttaggaggggagcagagaacttgaaagcatcttcctgttagtctttcttctcgtagacttcaaacttatacttgatgcctttttcctcctggacctcagagaggacgcctgggtattctgggagaagtttatatttccccaaatcaatttctgggaaaaacgtgtcactttcaaattcctgcatgatccttgtcacaaagagtctaaggtggcctggttgattcatggcttcctggtaaacagaactgcctccgactatccaaaccatgtctactttacttgccaattccggttgt

(19) United States
(12) Patent Application Publication
(10) Pub. No.: US 2007/0179094 A1
(43) Pub. Date: Aug. 2, 2007

(54) Modulation of MDL-1 activity for treatment of inflammatory disease

(75) Inventors: Daryl Faulds, Mill Valley, CA (US); Stefano Fiorucci, Perugia (IT); Ying-Ping Jiang, Lafayette, CA (US); Yi Liu, Tracy, CA (US); John Parkinson, Martinez, CA (US)

Correspondence Address: FOLEY AND LARDNER LLP;SUITE 500, 3000 K STREET NW, WASHINGTON, DC, 20007, US

(73) Assignee: BAYER SCHERING PHARMA AG, Berlin (DE)

(21) Appl. No.: 11/698,449

(22) Filed: Jan. 26, 2007

Related U.S. Application Data
(60) Provisional application No. 60/764,124, filed on Jan. 31, 2006.

(51) Int. Cl. A61K 38/17; C07H 21/04; C12P 21/06; C07K 14/705

(52) U.S. Cl. 514/12; 530/350; 435/69.1; 435/320.1; 435/325; 536/23.5

(57) ABSTRACT

The present invention provides compositions and methods that modulate MDL-1 activity in a cell, in vivo or in vitro. In particular, the present invention provides methods for treatment of inflammatory diseases using synthetic or recombinant compositions that modulate MDL-1 activity in a mammalian cell, in vivo or in vitro. More particularly, the present invention provides protein compositions useful for the treatment of diseases having an inflammatory process mediated by MDL-1.

This application claims the benefit of U.S. Provisional Patent Application No. 60/764,124, filed Jan. 31, 2006, which is incorporated by reference herein in its entirety.

### FIELD OF THE INVENTION

The present invention relates to compositions and methods that modulate MDL-1 activity in a cell, in vivo or in vitro. In particular, the present invention relates to the treatment of inflammatory disease using synthetic or recombinant compositions that modulate MDL-1 activity in a mammalian cell, in vivo or in vitro. More particularly, the present invention relates to protein compositions useful for the treatment of diseases having an inflammatory process mediated by MDL-1.

### BACKGROUND OF THE INVENTION

Macrophages and monocytes are critical in the regulation of immune responses. They are involved in various immune processes, including antigen presentation, chemokine and cytokine production, and inflammation (e.g., Gordon (1998) In: Paul W E. Ed. Fundamental Immunology, 4^{th} ed. Lippincott-Raven Publishers). The inflammatory activity of macrophages and monocytes is regulated by a variety of activating cell surface receptors with different structures and specificities.

The cell surface receptor DAP12 is predominantly expressed in resting human peripheral blood mononuclear cells, dendritic cells, peripheral blood monocytes and NK cell lines and clones (e.g., Lanier et al. 1998). Polycystic lipomembranous osterodysplasia with sclerosing leukoencephalopathy (PLOSL) or Nasu-Hakola disease is a recessively inherited disease characterized by a combination of psychotic symptoms rapidly progressing to presenile dementia and bone cysts restricted to wrists and ankles. Recent study by Paloneva's group has identified genetic mutations in both DAP12 and TREM-2 in PLOSL patients (e.g., Kiialainen et al. (2005) Neurobiol. Dis. 18:314-322). Thus, it has been suggested that the DAP12 signaling pathway in the monocyte/macrophage lineage may provide a link between lesions in the brain and bone cysts, and in the inflammatory process in disease.

For example, DAP12 deficient mice are reportedly resistant to EAE and that the resistance was associated with a significantly diminished production of IFN-γ due to reduced antigen-specific T cell proliferation, although the mechanism for this decrease was not explored directly (e.g., Bakker et al. (2000) Immunity 13:345-353). Further, DAP12 functionally deficient mice, generated by transgenic, nonfunctional DAP12 ITAM, reportedly altered innate immune responses (Tomasello et al. (2000) Immunity 13:355-364). Additionally, brain and bone damage, which is caused by defective microglial and osteoclast differentiation, were reported in DAP12 functional deficient mice (e.g., Nataf et al. (2005) Am. J. Pathol. 166:275-286).

Studies with DAP12 transgenic mice further implicate DAP12 as an important molecule in inflammation. Mice over-expressing DAP12 in both myeloid and lymphoid cells reportedly demonstrate a significant inflammatory syndrome associated with neutrophilia and lung infiltration by macrophages (e.g., Lukas et al. (2002) Eur. J. Immunol. 32:2653-2663). Thus, the DAP12 pathway may play a pivotal role in regulating the inflammatory process.

Myeloid expressing activating cell surface receptors that utilized the DAP12 pathway have been identified, e.g., the Signal-Regulatory Protein b1 (SIRP-b1) (e.g., Tomasello et al. (200a) Eur. J. Immunol. 30:2147-2156), and the Triggering Receptor Expressed on Myeloid Cells (TREM-1, 2 & 3) (e.g., Bouchon et al. (2000) J. Immunol. 164:4991-4995); Chung et al. (2002) Eur. J. Immunol. 32:59-66). The engagement of the activating receptor with an activating antibody has revealed functional roles of individual receptors in myeloid cells. The antibody cross-linking of TREM-1 induces monocyte secretion of pro-inflammatory chemokines and cytokines such as IL-8, MCP-1, and TNF-a, neutrophil degranulation, and upregulation of adhesion molecules such as CD40, CD86(B7-2), and CD54 (ICAM) on monocytes (e.g., Bouchon et al. (2001) Nature 410: 1103-1107). Engagement of TREM-2 receptor promotes upregulation of CCR7 receptor, partial dendritic cell (DC) maturation and survival (e.g., Bouchon et al. (2001) Nature 410: 1103-1107). Activation of SIRPb receptor triggers Fcg receptor-dependent and -independent phagocytosis in macrophages (e.g., Hayashi et al. (2004) J. Biol. Chem. 279: 29450-29460).

In particular, DAP12 reportedly associates with the cell surface receptor, the Myeloid DAP12-Associating Lectin (MDL-1), which may play an important role in the modulation of the inflammatory process, particularly, via association with DAP12. The association between MDL-1 and DAP12 is reportedly via ionic interaction between two oppositely charged amino acid residues, i.e., lysine in MDL-1 and aspartic acid in DAP12, located in the TM domains of these receptors (e.g., Bakker et al. (1999) Proc. Natl. Acad. Sci. USA 96:9792-9796). Further, the engagement of the MDL-1 receptor by a cognate ligand is proposed to result in phosphorylation of the ITAM tyrosine residues of DAP12 and recruitment of the SH2 domain-containing protein tyrosine kinase Syk to the receptor complex. Activation of the Syk kinase transduces signals to downstream signaling pathways (e.g., Tomasello et al. (2000a) Eur. J. Immunol. 30:2147-2156). The ITAM-bearing DAP12 adaptor protein therefore couples surface receptors to intracellular effectors, which induces a cascade of events leading to cell activation (FIG. 1).

MDL-1 is a type II transmembrane receptor first described in monocytes, macrophages and myeloid cell lines (Bakker et al. (1999) Proc. Natl. Acad. Sci. USA 96:9792-9796). Assignment of MDL-1 as a monocyte/macrophage activating immunoreceptor was based on indirect evidence via heterologous expression of the extracellular domains of CD69 fused to the intracellular, transmembrane and stalk domains of human MDL-1. In murine J774 macrophage cells, anti-CD69 cross-linking antibodies mediated an ion pair-dependent, non-covalent association of the CD69/MDL-1 chimera with the signal-transducing adaptor protein DAP12. The CD69/MDL-1 chimera recruited DAP12 to the cell surface and facilitated intracellular calcium mobilization (Bakker et al. (1999) Proc. Natl. Acad. Sci. USA 96:9792-9796). MDL-1 expression in mature myeloid cells was subsequently confirmed (Gingras et al. (2002) 38:817-824) and MDL-1 mRNA was also detected in human CD1a+ and CD14+dendritic cell (DC) subsets (Ahn et al. (2002) Blood 100:1742-1754). DAP12, MDL-1 and another DAP12-associating immunoreceptor, TREM-1, are co-expressed during Mycobacterium bovis-induced lung granuloma formation in mice (Aoki et al. (2004) Infect. Immun. 72:2477-2483). MDL-1 expression in granulomas was TNF-dependent and down-regulated by IFN-γ, suggesting a potential role for MDL-1 in regulating T-lymphocyte and macrophage-dependent granuloma formation, as has been established for TREM-1 (Nochi et al. (2003) 162:1191-1201). However, MDL-1 remains an orphan immunoreceptor with no known endogenous ligand and for which no surrogate ligand/agonist has been reported. Moreover, except for the findings of the present inventors described herein, there has been no known association of MDL-1 with a specific human disease or a defined functional role for MDL-1 in animal models of human disease.

MDL-1 belongs to the super family of Ca²⁺-dependent (C type) lectins. C type lectins are a family of glycoproteins that contain characteristic amino acid consensus sequences encoding evolutionarily conserved carbohydrate recognition domains (CRD) (e.g., Drickamer (1993) Curr. Opin. Struct. Biol. 3:393-400). CRDs bind to selected carbohydrates in a Ca2+ dependent manner; however, carbohydrate moieties do not necessarily serve as the only natural ligands for C type lectins. Some C type lectins recognize polypeptide sequences as their natural ligands such as the mouse NKG2D receptor, which binds to polypeptide sequences of its cognate ligands, H60 and RAE-1s, or the human NKG2D receptor and its ligand MICA (e.g., Cerwenka et al. (2000) Immunity 12:721-727; Diefenbach et al. (2000) Nature Immunol. 1:119-126; Li, et al. (2001) Nat. Immun. 2:443-451; Li et al. (2002) Immunity 16:77-86). Recently crystal structures of several C type lectin receptors have revealed that both peptide binding and carbohydrate binding receptors have a loop structure that allows the binding of their ligands. However, the loop in peptide binding receptor is shorter than that of carbohydrate binding receptor. It has been proposed that the shorter loop structure is no longer capable of accommodating the Ca2+ ion that is necessary for carbohydrate binding (e.g., Natarajan et al. (2000) Biochemistry 39:14779-14786; Li et al. (2002) Immunity 16:77-86).

MDL-1 may play an important role in mediating the inflammatory process in disease, particularly, via association with DAP12. The ability to modulate the inflammatory process by targeting key signaling components of that response pathway provide an important and specific means for treatment of inflammatory diseases e.g., for controlling undesirable or inappropriate physiological and developmental responses in the immune system, as described herein by the present inventors.

### SUMMARY OF THE INVENTION

The present invention provides compositions for modulating MDL-1 activity, and methods for treatment of inflammatory diseases using such compositions. More particularly, the present invention provides compositions and methods useful for treatment of a disease having an inflammatory process mediated by MDL-1. The present invention further provides compositions and methods for diagnosis, and monitoring the progression or treatment, of such inflammatory diseases. Examples of such diseases include, but are not limited to, multiple sclerosis (MS), inflammatory bowel disease (IBD), and arthritis.

In particular, the present invention provides compositions that modulate an MDL-1 activity in a mammalian cell, in vivo or in vitro, for example a human, primate, or rodent cell. In one aspect, the MDL-1 activity is the binding of MDL-1 to a cognate ligand, and in another aspect, the MDL-1 activity is the modulation of a DAP-12 activity. A composition of the present invention can modulate MDL-1 activity by, e.g., increasing or decreasing MDL-1 activity.

In some aspects, the composition of the present invention decreases MDL-1 activity. In one aspect, the decrease in MDL-1 activity results in a decrease in DAP-12 activity. In one aspect, the composition binds to an MDL-1 cognate ligand and, thereby, modulates MDL-1 activity. More particularly, in one aspect the composition binds to the MDL-1 binding site of an MDL-1 cognate ligand and, thereby, modulates MDL-1 activity.

In some aspects, the composition of the present invention is a synthetic or recombinant composition. In one aspect, the composition is a protein. In one aspect, the composition is a polyclonal or monoclonal antibody. In another aspect, the antibody decreases MDL-1 activity. In yet another aspect, the antibody increases MDL-1 activity.

In some aspects, the composition of the present invention is a fusion protein. In one aspect, the fusion protein comprises an Fc portion and an MDL-1 portion. In a particular aspect, the Fc-MDL-1 fusion protein of the present invention comprises a sequence encoding an Fc fragment and an amino acid sequence encoding an extracellular domain of MDL-1. Examples of such fusion proteins include, but are not limited to, the Fc-MDL-1 fusion protein encoded by: the amino acid sequence of SEQ ID NO: 1 (hFc-MDL-1); the nucleic acid sequence of SEQ ID NO: 2 (hFc-MDL-1); the amino acid sequence of SEQ ID NO: 3 (hFc-MDL-1); the nucleic acid sequence of SEQ ID NO: 4 (hFc-MDL-1); the amino acid sequence of SEQ ID NO: 5 (mFc-MDL-1); and the nucleic acid sequence of SEQ ID NO: 6 (mFc-MDL-1).

More particularly, examples of an Fc portion suitable for use in an Fc-MDL-1 fusion protein of the present invention includes, but is not limited to, the Fc portion encoded by: the amino acid sequence of SEQ ID NO: 7 (hFc); the nucleic acid sequence of SEQ ID NO: 8 (hFc); and the amino acid sequence of SEQ ID NO: 9 (mFc); and the nucleic acid sequence of SEQ ID NO: 10 (mFc). More particularly, examples of an MDL-1 portion suitable for use in an Fc-MDL-1 fusion protein of the present invention includes, but is not limited to, the MDL-1 extracellular domain (ECD) encoded by: the amino acid sequence of SEQ ID NO: 11 (hECD); the nucleic acid sequence of SEQ ID NO: 12 (hECD); and the amino acid sequence of SEQ ID NO: 13 (mECD); and the nucleic acid sequence of SEQ ID NO: 14 (mECD).

An MDL-1 of the present invention can comprise the full-length sequence of a wild-type or naturally occurring MDL-1, and MDL-1 like molecules, including fragments, analogs, variants, mutants, and derivatives MDL-1. Examples of an MDL-1 full-length sequence include, but are not limited to, the MDL-1 encoded by: the amino acid sequence of SEQ ID NO: 15 (hMDL-1); the nucleic acid sequence of SEQ ID NO: 16 (hMDL-1); and the amino acid sequence of SEQ ID NO: 17 (mMDL-1); and the nucleic acid sequence of SEQ ID NO: 18 (mMDL-1).

The present invention further provides pharmaceutical compositions for treatment of a disease in a patient. The pharmaceutical compositions comprise a composition of the present invention described herein and above for modulating MDL-1 activity. In some aspects, the patient is a mammal and, more particularly, is a human, primate, or rodent.

The present invention further provides methods for treatment of an MDL-1 mediated disease in a patient, using a pharmaceutical composition of the present invention. Examples of such diseases include, but are not limited to, multiple sclerosis (MS), inflammatory bowel disease (IBD), and arthritis. In some aspects, the patient is a mammal and, more particularly, is a human, primate, or rodent.

The present invention further provides methods for diagnosing, or monitoring the progression of or treatment of, an MDL-1 mediated disease in a patient, using a composition of the present invention. Examples of such diseases include, but are not limited to, multiple sclerosis (MS), inflammatory bowel disease (IBD), and arthritis. In some aspects, the patient is a mammal and, more particularly, is a human, primate, or rodent.

The present invention further provides uses of a composition of the present invention in the manufacture of a medicament for treatment of an MDL-1 mediated disease in a patient. Examples of such diseases include, but are not limited to, multiple sclerosis (MS), inflammatory bowel disease (IBD), and arthritis. In some aspects, the patient is a mammal and, more particularly, is a human, primate, or rodent.

The present invention further provides compositions for diagnosing, and/or monitoring the progression or treatment of, an MDL-1 mediated disease in a patient, using a composition of the present invention that targets MDL-1 and/or an MDL-1 binding partner (e.g., and MDL-1 ligand or DAP12). Examples of such compositions include, but are not limited to a diagnostic or therapeutic imaging agent comprising a targeting moiety directed to MDL-1 and/or an MDL-1 binding partner (e.g., an anti-MDL-1 polyclonal or monoclonal antibody, and/or Fc-MDL-1 fusion protein of the present invention).

The present invention further provides kits for diagnosing, or monitoring the progression or treatment of, an MDL-1 mediated disease in a patient, using a composition of the present invention, e.g., an anti-MDL-1 polyclonal or monoclonal antibody, and/or Fc-MDL-1 fusion protein of the present invention. Examples of such diseases include, but are not limited to, multiple sclerosis (MS), inflammatory bowel disease (IBD), and arthritis. In some aspects, the patient is a mammal and, more particularly, is a human, primate, or rodent.

The present invention also provides expression vectors comprising a sequence encoding a composition of the present invention that modulates MDL-1 activity as described herein. In some aspects, the expression vector encodes a fusion protein and, more particularly, an Fc-MDL-1 fusion protein. Examples of such vectors include, but are not limited to, the vector exemplified in the nucleic acid sequence of SEQ ID NO: 19, encoding a human Fc-MDL-1 fusion protein; and the nucleic acid sequence of SEQ ID NO: 20, encoding a mouse Fc-MDL-1 fusion protein.

The present invention also provides cells comprising an expression vector encoding a protein of the present invention and expresses the encoded protein. In some aspects, the cell is a mammalian cell and, more particularly, a Chinese Hamster Ovary (CHO) cell. In a particular aspect the cell comprises the vector exemplified in the nucleic acid sequence of SEQ ID NO: 19, encoding a human Fc-MDL-1 fusion protein and expresses the fusion protein; and the nucleic acid sequence of SEQ ID NO: 20, encoding a mouse Fc-MDL-1 fusion protein and expresses the fusion protein.

### BRIEF DESCRIPTION OF THE DRAWINGS

The foregoing and other objects of the present invention, the various features thereof, as well as the invention itself may be more fully understood from the following description, when read together with the accompanying drawings in which:

FIG. 1 illustrates a non-limiting example of a signaling mechanism of MDL-1 and DAP12, where MDL-1 associates with DAP12 through the interaction between lysine and aspartic acid residues.

FIG. 2 illustrates a sequence alignment between MDL-1 and other C-type lectin proteins. Conserved residues are in bold, and conserved cystein residues are in italic. The predicted trans-membrane (TM) regions are underlined and the charged lysine residues in the TM region are in bold and underlined. hMDL-1 (human MDL-1); mMDL-1 (mouse MDL-1)

FIG. 3 illustrates human MDL-1 protein expression during macrophage differentiation, where (A) is a Western blot analysis of the time course of human MDL-1 protein expression after treatment with M-CSF (20 ng/ml); (B) is a FACS analysis of hMDL-1 surface expression after 7 days of treatment with M-CSF; (C) is IHC staining of human macrophages with preimmune and α-hMDL-1 antibody.

FIG. 4 illustrates the expression of mMDL-1 in a murine chronic EAE model, where (A) is a Northern blot analysis of mMDL-1 expression at different times during the course of mcEAE (and total RNA was extracted from spinal cords and β-actin was used as an RNA loading control); (B) is the quantitative analysis of mMDL-1 mRNA expression level, indicated as fold change relative to background.

FIG. 5 illustrates the interaction between human MDL-1 and DAP12, where (A) is a Western blot analysis of human MDL-1 proteins (20 ug of total cell lysates from untransfected HEK293 (lane 1) and human MDL-1/DAP12 transfected 293 (lane 2), and 0.5 ng and 2.5 ng (lanes 3 and 4) of baculovirus expressed hMDL-1ECD were run on SDS-PAGE and blotted with rabbit α-hMDL-1 Abs; (B) is the immunoprecipitation of human MDL-1 and DAP12 (where lysates from untransfected 293 cells (lane 1) and human MDL-1/DAP12 transfected 293 cells (lane 2) were incubated with rabbit α-hMDL-1 Abs and blotted with α-human DAP12 Abs. LC (IgG light chain)

FIG. 6 illustrates one non-limiting example of a serotonin release assay where ITAM in both Fcε1γ and DAP12 transduce intracellular signals through the Syk kinase pathway.

FIG. 7 illustrates the specificity of the serotonin release assay where (A) shows the results of parental cells and transfectants incubated with ³H serotonin for 20 hours (The cells were incubated with antibodies or ionophore (loading control) for 20 min. Supernatants were collected for determination of ³H serotonin.) RPMI (cell culture medium), CL (IgEmAb cross-linker), Ca Iono (Ca ionophore), IgG control (rabbit IgG control Abs), IgG α-hMDL-1 (rabbit α-hMDL-1 Abs); and (B) shows the results of rabbit α-hMDL-1 Abs preincubated with different concentrations of hMDL-1-Fc or mMDI-1-Fc for 20 min. (Antibodies were added to RBL transfectants for serotonin release.).

FIG. 8 illustrates TNFα production in human macrophages after activation by α-hMDL-1 Abs where (A) is TNFα production in monocytes and macrophages (CD14⁺ monocytes were treated with LPS and different Abs. Macrophages were differentiated from monocytes with GM-CSF treatment for 6 days. Cells were treated with LPS and Abs for 3 hrs. Supernatants were tested with ELISA. PI (protein A agarose purified rabbit preimmune IgG), α-MDL-1 (was protein A agarose purified α=hMDL-1 IgG); and (B) is the time course of TNFα production in macrophages (Macrophages were treated with antibodies for 3 hrs.).

FIG. 9 illustrates the FACS analysis of rabbit α-mMDL-1 Ab in mMDL-1/mDAP12-293 cell transfectants. PI (protein A purified rabbit preimmune sera), Ab (Protein A purified rabbit α-mMDL-1 Ab)

FIG. 10 illustrates IHC staining of mouse MDL-1, where (A) shows mMDL-1/mDAP12-293 cell transfectants that were grown on glass chamber slides (The cells were fixed and stained with rabbit preimmune and α-mMDL-1 sera.); and (B) shows mMDL-1/mDAP12-293 cell transfectants paraffin-embedded and stained with different antibodies.

FIG. 11 illustrates a non-limiting example of β-galactosidase reporter system in a BWZ cell line, where (A) shows that IL-2 and β-galactosidase production in a BWZ cell line can be induced by activation via the CD3ζ chain or DAP12; and (B) shows MDL-1 specific signaling in a CD3zH912Z1 stable cell line.

FIG. 12 illustrates the activation of BWZ transfected reporter cells with α-MDL-1 pAb (The stable bulk line expressing both the CD3ζMDL1 chimera and DAP12 (CD3zH912Z1) was able to signal specifically upon ligation with α-MDL-1. Both the chimera only transfected line (CD3zMDL1) and the line transfected with the full-length MDL-1 and DAP12 (V5H9/DAP12) were comparable to the parental line (BWZ)).

FIG. 13 illustrates that soluble MDL-1 protein inhibits α-hMDL-1 pAb activation of CD3zH912Z1 (Exogenous proteins included human MDI-1 extracellular domain protein expressed in baculovirus (hMDL1-ECD baculo). Murine MDL-1 ECD fused to IgG Fc domain expressed in CHO cells (mMDL-1Fc). Human Trem2-ECD fused to IgG Fc domain and expressed in baculovirus (hTREM2-Fc baculo), and human ICAM fused to IgG Fc domain and expressed in baculovirus (ICAM-Fc baculo).

FIG. 14 illustrates the MDL-1 fusion protein constructs, where (A) is a schematic diagram an non-limiting example of an MDL-1 fusion protein and of MDL-1; TM (MDL-1 transmembrane domain), ECD (MDL-1 extracellular domain), VH (IgG1 heavy chain variable region), CH (IgG1 heavy chain constant region); (B) is a schematic diagram of an unlimiting example of an expression vector, pPEP1.hFchH9 (9282 bp), which encodes and expresses human MDL-1/Fc fusion protein (hFc-hMDL-1); and (C) illustrates the amino acid sequence of hFc-hMDL-1 (The first letter “A” (blue) at the beginning of the sequence is an additional alanine due to signal cleavage, the bolded (black) letters (following the first letter “A”) is the sequence of human Fc, and the unbolded (green) letters (following the bolded letters of the human Fc sequence) is the sequence of human MDL-1 extracellular domain (ECD).

FIG. 15 Illustrates in (A) a non-limiting example for blocking MDL-1 function with an MDL-1 decoy protein, where in (1) activation of MDL-1 releases pro-inflammatory chemokines and cytokines; and (2) MDL-1 fusion protein binds to MDL-1 ligand and blockis MDL-1 activity or function (although MDL-1 ligand had not been identified, the ligand can be for example, soluble or membrane bound as illustrated in (1) and (2);

and in (B) the properties of mFc-mMDL-1 fusion protein, where (1) is an SDS-PAGE analysis of the fusion protein at natured (left panel) and denatured (right panel) conditions; (2) illustrates the results of a FACS analysis to validate the inhibitory activity of the fusion protein. (α-mMDL-1 Ab was incubated with different concentration of fusion protein for 30 min, and the mixed solution was then added to 293-mMDL-1/mDAP12 cells and analyzed by FACS.).

FIG. 16 illustrates the pharmacokinetic study of mFc-mMDL fusion protein, where the fusion protein at 100 ug/mouse was administered i.v. or i.p. to SJL mice, and the fusion protein was measured by ELISA assay.

FIG. 17 illustrates MDL-1 expression in Crohn's disease and colitis tissue, where (A) illustrates the results of an RT-PCR analysis of human MDL-1 expression in Crohn's disease tissues; and (B) illustrates the results of an RT-PCR analysis of mouse MDL-1 expression in TNBS-induced colitis tissues. *(P<0.05 versus control)

FIG. 18 illustrates human MDL-1 in Crohn's disease (A) and Ulcerative colitis (B) tissues, where left panels are H and E staining of normal and disease tissues at 10× magnification; right panels are IHC staining of MDL-1 of normal and disease tissues at 60× magnification. In the IHC staining, a total of 12 CD tissues (9 SI and 3 colon tissues) with transmural inflammation, ulceration, and granuloma formation were stained with similar result. A total of 4 UC tissues with mucosal ulceration, chronic active inflammation, crypt abscesses, and follicle formation were stained with similar result. A total of 8 normal tissues (4 SI and 4 colon tissues) are used as control in IHC staining. The arrows in the lower right panels of both (A) and (B) represent the membrane staining of MDL-1.

FIG. 19 illustrates in (A) IHC staining of mouse MDL-1 in normal colonic tissue; in (B) and (C) IHC staining of mouse MDL-1 in colitis colonic tissue of TNBS-induced colitis mouse; and in (D) IHC staining of MDL-1 in colitis colonic tissue of mouse treated with mFc-mMDL-1 fusion protein. The dark spots represent the staining of mMDL-1.

FIG. 20 illustrates that mFc-mMDL-1 reduces mortality in TNBS colitis, where colitis was induced in Balb/c mice by intrarectal administration of 2 mg/mous TNBS. Mouse control IgG1 (cIgG1) and mFc-mMDL-1 fusion protein (FP) were administrated intraperitoneally at day 0. Mice were followed for 7 days. P<0.05 FP treated versus TNBS. n=10 mice per group.

FIG. 21 illustrates that mFc-mMDL-1 reduces mortality in a mild colitis model (with 0.5 mg TNBS/mouse).

FIG. 22 illustrates that mFc-mMDL-1 attenuates mouse TNBS colitis, where in (A) disease scores are presented as the weight of animal at day 7; (B) is the disease activity index; (C) is the histology score; and (D) I the MPO activity. The data represent mean±SEM; n=10; *(P<0.05 versus control); and **(P<0.05 versus TNBS).

FIG. 23 illustrates in (A) that mFc-mMDL-1 reduces the level of proinflammatory cytokine, TNF-α, in both plasma and intestinal mucus; in (B) that mFc-mMDL-1 reduces the level of inflammatory mediators in intestinal mucus; and in (C) the results of a quantitative RT-PCR (qRT-PCR) analysis of cytokine and chemokine in intestinal mucus; n=10; *(P<0.05 versus control); and **(P<0.05 versus TNBS).

FIG. 24 illustrates that in (A) mFc-mMDL-1 reduces mortality similar to prednisolone treatment in TNBS colitis; and in (B) mFc-mMDL-1 fusion protein attenuates TNBS colitis at a similar degree as Prednisolone.

FIG. 25 illustrates that in (A) the mortality in DSS-induced colitis;

in (B) mFc-mMDL-1 fusion protein in DSS colitis, wherein in (1) mFc-mMDL-1 fusion protein attenuates DSS induced colitis (Colitis was induced in Balb/c mice by 5% DSS added to the drinking water. Control IgG1 (cIgG1) and fusion protein (FP) were administrated i.p at day 0; and in (2) mFc-mMDL-1 fusion protein reduces the production of inflammatory mediators in intestinal mucus;

in (C) the results of a quantitative RT-PCR (qRT-PCR) analysis of cytokine and chemokine in intestinal mucus; and

in (D) the histopathological analysis of DSS-induced colitis.

FIG. 26 illustrates in (A) that the DAP12 expression level affects the macroscopic and microscopic scores and MPO levels of mouse colitis. Wt (wild type), DAP12−/− (DAP12 knock-out mice), DAP12tg (DAP12 transgenic mice), n=6-12, *(P<0.05 versus control), **(P<0.05 versus DSS);

in (B), that DAP12 expression levels affects mortality in DSS colitis;

in (C), DSS-induced colitis in DAP12−/− and DAP12tg mice;

in (D), quantitative RT-PCR (qRT-PCR) analysis of cytokine and chemokine in intestinal mucus.

Wt (wild type), DAP12−/− (DAP12 knock-out mice), DAP12tg (DAP12 transgenic mice), n=6-12, *(P<0.05 versus control), **(P<0.05 versus DSS)

FIG. 27 illustrates in (A) and (B) the results of treatment of TNBS colitis with mFc-mMDL-1, where (A) shows the change of body weight (Colitis was induced in Balb/c mice by intrarectal administration of 1 mg/mouse TNBS. Treatment was started on day 5 after administration of the TNBS. The animals were followed for 14 days.) n=8-12; and (B) shows the disease scores. *(P<0.05 versus control), **(P<0.05 versus TNBS);

in (C) that treatment of TNBS colitis with mFc-mMDL-1 reduces mortality;

in (D) that mFC-mMDL-1 treats TNBS induced colitis; and

in (E) that treatment of mFc-mMDL-1 reduces the level of proinflammatory mediators in TNBS colitis.

### DETAILED DESCRIPTION OF THE INVENTION

The present invention provides novel compositions and methods for treatment of inflammatory diseases, particularly those mediated by Myeloid DAP12-associating lectin (MDL-1). The present invention further provides compositions and methods for diagnosis, and monitoring the progression or treatment, of such inflammatory diseases. Examples of such diseases include, but are not limited to, multiple sclerosis (MS), inflammatory bowel disease (IBD), and arthritis.

The present inventors demonstrate herein that MDL-1 is an attractive and innovative therapeutic target for the treatment of inflammatory diseases, particularly in view of the role of MDL-1 in macrophage-mediated inflammation and its association with inflammatory diseases. MDL-1, is a surface protein expressed primarily in myeloid cells. A direct interaction between MDL-1 and the adaptor protein DAP12 is reportedly necessary for its signaling function. The data provided herein demonstrate a direct interaction between MDL-1 and DAP12, and further demonstrate a direct correlation between an increase in MDL-1 expression and clinical disease score in animal models. Additionally, the data provided herein demonstrate that activation of MDL-1 with specific antibodies generated against MDL-1 triggers the release of a variety of pro-inflammatory chemokines and cytokines from macrophages. Further, as described herein, MDL-1 expression is found in brain pons and choroid plexus from a patient with Hungtington's disease, brain hippocampus from a patient with multiple microinfarcts (Incyte database), and in disease tissue of MS and IBD. Thus, these findings strongly implicate MDL-1 in inflammatory processes.

Additionally, the present inventors have generated and characterized anti-MDL-1 antibodies and MDL-1 fusion proteins for FACS and IHC analysis. As described herein, MDL-1 expression can be studied by IHC and FACS in both the CNS and the periphery to further define the role of MDL-1 in inflammatory disease pathology, e.g., in MS, IBD, and arthritis. As shown in the Examples herein, the expression of MDL-1 can be tested by IHC and FACS in the tissue of patients with an inflammatory disease. Such studies provide critical information on the role of MDL-1 and its ligand in such inflammatory diseases and as therapeutic targets for treatment of MDL-1 mediated inflammatory diseases.

Further, both pharmacological and genetic approaches demonstrated herein by the present inventors establish that MDL-1 and its signal-transducing adaptor protein DAP-12 contribute to the pathology of two experimental colitis models with distinct etiologies (DSS and TNBS colitis). In particular, the present inventors demonstrate herein that: 1) DAP-12^{-/-} mice are protected from colitis whereas overexpressing DAP-12 transgenic mice have exacerbated disease; 2) a mouse MDL-1 fusion decoy protein (mFc-MDL-1) ameliorates disease; 3) mFc-MDL-1 efficacy correlates to decreased mucosal TNF-α, MCP-1 and IFN-γ; 4) mFc-MDL-1 prevents colitis onset and treats established colitis and due to a robust PK profile provides efficacy when given as a single dose in 7 to 14 day studies. Thus, the MDL-1 fusion protein is effective in both prophylactic and therapeutic treatment regimens, and the action of MDL-1 fusion protein may be mediated by reducing the production of pro-inflammatory chemokines and cytokines.

Taken together, the findings of the present inventors as described herein, indicate that MDL-1 plays an important role in the pathogenesis of inflammatory diseases, and that MDL-1 is a novel therapeutic target for treatment of such inflammatory diseases. Based on these findings, the present invention provides novel pharmaceutical compositions that modulate MDL-1 activity, and methods for the treatment of inflammatory diseases (e.g., MS, IBD, and arthritis), using such compositions. In one embodiment, the compositions and methods of the present invention inhibit the activity of MDL-1 and, thereby, modulate and, more particularly, decrease or inhibit an inflammatory process or response (e.g., myeloid cell activation).

Abbreviations

α-hMDL-1 Abs (Rabbit anti-human MDL-1 polyclonal antibodies)

α-mMDL-1 Abs (rabbit anti-mouse MDL-1 polyclonal antibodies)

aAb or ACT Ab (anti-MDL-1 activating antibody)

AS linker (alanine and serine linker between fusion protein)

5-ASA (5-aminosalicylic acid)

CD (Crohn's disease)

CH2 (IgG constant domain 2 of the heavy chain)

CHO (chinese hamster ovary cell)

DAP12 (DNAX activation protein MW12,000 kDa)

DAP12tg (DAP12 transgenic mice)

DAP-12^{-/-} (DAP12 knock out mice)

DG44 (dhfr-deficient CHO cell)

DHFR (dihydrofolate reductase)

DSS (dextran sulfate sodium)

DXB11 (dhfr-deficient CHO cell)

EAE (experimental autoimmune encephalomyelitis)

ECD (extracellular domain)

ELISA (enzyme linked immunosorbent assay)

FACS (fluorescence-activated cell sorting)

Fc (constant fragment or fragment from constant region)

FcεRI or Fc epsilon RI (IgE receptor)

FFF-LALS (field flow fractionation-Low angle light scattering)

Fig. (figure)

Figs. (figures)

GLP (good laboratory practice)

GMP (good manufacturing practice)

hECD (human ECD)

HEK293 (human embryonic kidney 293 cell)

hFc (human Fc)

hFc-MDL-1 (human Fc-MDL-1)

hMDL-1 (human MDL-1)

HPLC-SEC (high performance liquid chromatography-Size exclusion chromatography)

IBD (inflammatory bowel disease)

IFN (interferon)

IFN-γ or IFN-g (interferon γ)

IHC (immunohistochemistry)

IL-2 (interleukin-2)

IP (immunoprecipitation)

ITAM (immunoreceptor tyrosine-based activation motif)

LFA-3 (leukocyte function antigen-3)

LPS (lipopolysaccharide)

MDL-1 (myeloid DAP12-Associating lectin)

MCP-1 (macrophage chemoattractant protein 1)

mFc (mouse Fc)

mFc-MDL-1 (mouse Fc-MDL-1)

mMDL-1 (mouse MDL-1)

MIP-1α (macrophage inflammatory protein 1α)

MPO (myeloperoxidase)

MS (multiple sclerosis)

MTX (methotrexate)

PK (pharmacokinetics)

PLOSL (polycystic lipomembranous osterodysplasia with sclerosing leukoencephalopathy)

RBL-2H3 (rat basophilic leukemia cell

RBL-hMDL-1/hDAP12 (RBL-2H3 transfectant stably expressing both human MDL-1 and DAP12)

PMA (phorbol 12-myristate 13-acetate)

SDS-PAGE (SDS polyacrylamide gel electrophoresis)

SH2 domain (Src homology 2 domain)

SIRPα1 (signal-regulatory protein α1)

Syk kinase (spleen tyrosine kinase)

THP-1 (human monocyte-like cell)

TM (transmembrane)

TNBS (trinitrobenzene sulfonic acid)

TNF-α or TNF-a (tumor necrosis factor α)

TNFR (tumor necrosis factor receptor)

TREM (triggering receptor expressed on myeloid cells)

WB (western blot)

UV (ultraviolet)

Definitions

‘A’ and ‘an’, as used herein, means ‘one or more’ unless otherwise specified.”

“Activity” as used herein with reference to MDL-1 activity, refers to a cellular, biological, and/or therapeutic activity or function of an MDL-1 of the present invention. Examples of such activities include, but are not limited to, signal transduction, interacting or associating with an MDL-1 ligand or other binding partner (e.g., DAP12) or cellular component, modulating an inflammatory response or process, including myeloid cell activation.

“Amino acid” as used herein with reference to an amino acid sequence or composition encompasses a protein (e.g., a protein sequence or composition).

“Disease” as used herein encompasses a stage, symptom, condition, or pathology of a disease, or genetic predisposition for a disease. An example of a disease is an inflammatory disease, e.g., MS, IBD, or arthritis, and encompasses a state or condition of inflammation.

“MDL-1 mediated” as used herein with reference to a disease, refers to an activity or signal transduction pathway of MDL-1 (or an activity or signal transduction pathway that is mediated by MDL-1). Thus, an MDL-1 mediated disease is a disease where an MDL-1 activity or signal transduction pathway can be modulated for treatment of the disease. More particularly, the present invention encompasses compositions and methods that modulate an MDL-1 activity and/or signal transduction pathway to provide a therapeutic benefit or therapeutic activity for treatment of a disease, e.g., an inflammatory disease.

“Modified” as used herein, with reference to a composition of the present invention refers to any reaction or manipulation resulting in a change or alteration of a reference nucleic acid, amino acid, or chemical molecule to arrive at a desired composition or molecule of the present invention (e.g., mutation of a wild-type protein or nucleic acid to arrive at a desired variant thereof having a biological and/or therapeutic activity; mutation of a protein or nucleic acid sequence to arrive at a desired humanized sequence; or alteration of a chemical compound to arrive at a desired chemical structure, and/or biological and/or therapeutic activity. In particular, the compositions of the present invention can be modified or optimized to achieve a particular biological or therapeutic activity or a particular specificity (e.g., specific to a particular tissue, condition, disease, or binding partner or other cellular component).

“Modulate” as used herein with reference to MDL-1 activity refers to a change in MDL-1 activity and can be an increase or decrease in MDL-1 activity, as compared to a wild-type or naturally-occurring MDL-1.

“Nucleic acid” as used herein with reference to a nucleic acid sequence or composition of the present invention encompasses a nucleic acid molecule, e.g., a DNA or RNA, or fused, chimeric, modified, isolated, synthetic, or recombinant form thereof. Examples of suitable nucleic acids include, but are not limited to, a wild-type, full-length DNA or RNA (e.g., mRNA) encoding a protein, or other nucleic acid molecule having a biological or therapeutic activity (e.g., shRNA, siRNA, ribozyme, antisense RNA or DNA, RNA or DNA oligonucleotide) or encoding a protein having a biological or therapeutic activity, or an analog, derivative, or variant thereof. More particularly, nucleic acid variants of the present invention can be muteins, i.e., comprising a mutation, e.g., a single or multiple nucleic acid substitution, deletion, or addition such that the varian retains or has a biological or therapeutic activity.

As used herein, “patient” refers to a subject, more particularly a mammal (e.g., a human, non-human primate, or rodent), and even more particularly, a mammal in need of treatment for a disease.

“Protein” or “amino acid” used herein with reference to an amino acid sequence or composition of the present invention encompasses a peptide, full-length protein, or fragment or portion of a full-length protein. Further, a protein of the present invention can be a fused, chimeric, heterologous, modified, isolated, synthetic, or recombinant amino acid molecule. In particular, examples of suitable proteins include, but are not limited to, a wild-type, full-length protein (including a secreted or soluble form thereof), or an analog, derivative, functional equivalent, or biologically active form, thereof. More particularly, protein variants of the present invention can be muteins (or mutants), ie.e., comprising a mutation e.g., a single or multiple amino acid substitution, deletion, or addition such that the variant retains or has a biological or therapeutic activity. Sequences encoding a protein may include, e.g., codon-optimized version fo wild-type protein sequences, or humanized sequences. Optimal codon usage in humans can be identified from codon usage frequencies for expressed human genes and may be determined by methods known in the art, e.g., program “Human High.codN” from the Wisconsin Sequence Analysis Package, version 8.1, Genetics Computer Group, Madison, Wis. For example, codons that are most frequently used in highly expressed human genes may be optimal codons for expression in the cells of a human subject and, thus, can be used as a basis for constructing a synthetic coding system.

“Inflammatory process” as used herein may be used interchangeably with “inflammatory response”.

“Pharmaceutical composition” as used herein refers to a composition having a therapeutic benefit or therapeutic activity for treatment of a disease.

“Subject” as used herein refers to a mammal, e.g., a human, non-human primate, or rodent.

“Targeting moiety” as used herein with reference to a diagnostic and/or therapeutic composition of the present invention, and more particularly a diagnostic and/or therapeutic imaging agent, refers to a composition of the present invention that directly or indirectly binds to, interacts with, or otherwise associates with or is directed to MDL-1 and/or an MDL-1 binding partner (e.g., an MDL-1 ligand or DAP12). Examples of such targeting moieties include, but are not limited to, an anti-MDL-1 antibody, MDL-1 fusion protein, and more particularly an MDL-1 competitor or decoy fusion protein.

“Therapeutic benefit” or “therapeutic activity” as used herein refers to an effect which provides a therapeutic benefit to a patient for a disease (including a stage, symptom or condition of a disease), e.g., slowing, inhibiting, or preventing the onset or progression of a disease, stabilizing or ameliorating a disease.

“Treatment”, “treating”, “treat”, or grammatical equivalents thereof, refers to providing a therapeutic benefit to a patient for a disease, including a stage, symptom or condition of a disease, including the slowing, inhibiting, or preventing the progression of a disease, ameliorating a disease, or having a beneficial consequence for a subject treated (e.g., a patient).

Technical and scientific terms used herein have the meanings commonly understood by one of ordinary skill in the art to which the present invention pertains, unless otherwise defined. Reference is made herein to various methodologies known to those of ordinary skill in the art. Publications and other materials setting forth such known methodologies to which reference is made are incorporated herein by reference in their entireties as though set forth in full. Standard reference works setting forth the general principles of recombinant DNA technology include Sambrook, J., et al. (1989) Molecular Cloning: A Laboratory Manual, 2d Ed., Cold Spring Harbor Laboratory Press, Planview, N.Y.; McPherson, M. J. Ed. (1991) Directed Mutagenesis: A Practical Approach, IRL Press, Oxford; Jones, J. (1992) Amino Acid and Peptide Synthesis, Oxford Science Publications, Oxford; Austen, B. M. and Westwood, O. M. R. (1991) Protein Targeting and Secretion, IRL Press, Oxford. Any suitable materials and/or methods known to those of ordinary skill in the art can be utilized in carrying out the present invention. However, preferred materials and methods are described. Materials, reagents and the like to which reference is made in the following description and examples are obtainable from commercial sources, unless otherwise noted.

The references cited herein, including patents, patent applications and journal articles, are incorporated herein by reference, in their entirety.

Compositions

The present invention provides compositions that modulate MDL-1 activity in a cell, in vivo or in vitro. In particular, the present invention provides compositions that modulate MDL-1 activity and are useful as pharmaceutical compositions for treatment of inflammatory disease in a patient (e.g., a human, non-human primate, or rodent). More particularly, the present invention provides protein compositions that modulate MDL-1 and are useful as pharmaceutical compositions for the treatment of diseases having an inflammatory process mediated by MDL-1. Preferably, the patient is a mammal (e.g., human, non-human primate, or rodent) and more preferably, the patient is a human.

The compositions of the present invention can modulate MDL-1 activity directly or indirectly. For example, a composition of the present invention can modulate MDL-1 activity in a cell, by interacting with, associating with, or binding to MDL-1 and/or a ligand of MDL-1. Further, MDL-1 activity can be modulated, e.g., by increasing or decreasing the quantity, binding affinity, level of activity, and/or other characteristics of a composition of the present invention. More particularly, a composition of the present invention can modulate MDL-1 activity by disrupting or enhancing signal transduction by MDL-1 e.g., by disrupting or enhancing the formation of or activity of the DAP12/MDL-1 complex and/or MDL-1/MDL-1 ligand complex.

In particular embodiments, the compositions of the present invention include e.g., competitive inhibitors or antagonists of MDL-1 activity and/or MDL-1 ligand activity, that decrease MDL-1 activity in a cell, in vitro or in vivo. Examples of such compositions include, but are not limited to, an anti-MDL-1 polyclonal or monoclonal antibody, Fc-MDL-1 fusion protein that functions as a decoy protein or competitor for MDL-1 ligand, soluble MDL-1 receptor or other MDL-1-like molecule that decreases MDL-1 activity in a cell.

In other embodiments, the compositions of the present invention include, e.g., activators or agonists of MDL-1 activity and/or MDL-1 ligand activity, that increase MDL-1 activity in a cell, in vitro or in vivo. Examples of such compositions include, but are not limited to, an anti-MDL-1 polyclonal or monoclonal antibody, other protein or MDL-1 like molecule that increases MDL-1 activity in a cell, in vitro or in vivo. An MDL-1 “activation antibody” or “activating antibody” (aAb or ACT Ab) as used herein is an antibody that increases MDL-1 activity in a cell, in vitro or in vivo.

Another embodiment of the present invention relates to a soluble MDL-1 that modulates MDL-1 activity in a cell, in vitro or in vivo. For example, in a particular embodiment, a soluble MDL-1 receptor is an MDL-1 protein lacking the MDL-1 transmembrane domain. In a particular embodiment, the soluble MDL-1 receptor is a competitive inhibitor or antagonist of MDL-1 activity. Examples of a soluble MDL-1 receptor includes, but is not limited to the protein encoded by: the amino acid sequence of SEQ ID NO: 11 (hMDL-1 ECD); the nucleic acid sequence of SEQ ID NO: 12 (hMDL-1 ECD); the amino acid sequence of SEQ ID NO: 13 (mMDL-1 ECD); and the nucleic acid sequence of SEQ ID NO: 14 (mMDL-1 ECD).

Suitable compositions of the present invention are synthetic or recombinant compositions and further nucleic acid, amino acid, or chemical compositions. In particular, in some embodiments, the composition is a protein, e.g., a polyclonal or monoclonal antibody and more particularly, a fusion protein. Suitable compositions of the present invention also encompass a heterologous, chimeric, or hybrid protein. Biologically-active fragments, analogs, variants, conjugates, mutants (or muteins), and derivatives of a wild-type or naturally-occurring protein are also encompassed.

A fusion protein of the present invention has at least two portions fused together directly or indirectly (e.g., via a linker). Further, an advantage of a fusion protein of the present invention, has an extended or increased serum half-life, stability, and/or biological activity, e.g., as compared to a naturally-occurring or wild-type protein. For example, and advantage of an MDL-1 fusion protein of the present invention, e.g., an Fc-MDL-1 fusion protein, is that it has an increased serum half-life and/or stability in a cell, as compared to a naturally-occurring or wild-type protein. Such fusion proteins of the present invention can be made using known methods, e.g., via chemical modification.

Chemical modification of biologically active peptides, proteins, oligonucleotides and other drugs for purposes of extending the serum half-life of such bioactive agents has been extensively studied. The ability to extend the serum half-life of such agents allows for the therapeutic potential of the agent to be realized without the need for high dosages and frequent administration, which is a further advantage of the compositions of the present invention.

Examples of chemical modifications used to extend the half-lives of proteins in vivo include, but are not limited to, the chemical conjugation of a water soluble polymer, such as polyethylene glycol (PEG), to the protein of interest. A variety of approaches have been used to attach the polyethylene glycol molecules to the protein (PEGylation) (see e.g., U.S. Ser. No. 09/817,725 and EP1564219 regarding N-terminally monopegylated polypeptides and a process for their preparation; U.S. Pat. No. 4,002,531 regarding reductive alkylation used for attachment of polyethylene glycol molecules to an enzyme; U.S. Pat. No. 4,179,337 regarding PEG:protein conjugates involving, e.g., enzymes and insulin; U.S. Pat. No. 4,904,584 regarding the modification of the number of lysine residues in proteins for the attachment of polyethylene glycol molecules via reactive amine groups; U.S. Pat. No. 5,834,594 regarding substantially non-immunogenic water soluble PEG:protein conjugates, involving for example, the proteins IL-2, interferon alpha, and IL-1ra which disclose the use of unique linkers to connect the various free amino groups in the protein to PEG; and U.S. Pat. Nos. 5,824,784 and 5,985, 265 regarding methods allowing for selectively N-terminally chemically modified proteins and analogs thereof, including G-CSF and consensus interferon).

Other approaches designed to extend the serum half-life of bioactive agents include: conjugation or fusion of a biologically active protein (e.g., MDL-1 or fragment thereof) to a large, stable protein which is too large to be filtered through the kidneys (e.g., serum albumin); use of low- and high-density lipoproteins as transport vehicles and to increase serum half-life; or the use of the Fc domain of immunoglobulins to produce Fc-fusion proteins (see e.g., G. D. Mao et al., Biomat., Art. Cells, Art. Org. 17:229-244 (1989); Smidt et al. (1991) Nuc. Acids. Res. 19(17):4695-4700; PCT WO 98/28427, and PCT WO 00/24782).

In some embodiments, the fusion protein is an Fc-fusion protein, e.g., and Fc-MDL-1 fusion protein, having extended or increased serum half-life, stability, and/or biological activity, e.g., as compared to a naturally-occurring or wild-type MDL-1. Immunoglobulins of IgG class are among the most abundant proteins in human blood. Their circulation half-lives can reach as long as 21 days. Fusion proteins have been reported to combine the Fc regions of IgG with the domains of another protein, such as various cytokines and soluble receptors (see, e.g., Capon et al., Nature, 337:525-531, 1989; Chamow et al., Trends Biotechnol., 14:52-60, 1996); U.S. Pat. Nos. 5,116,964 and 5,541,087). The prototype fusion protein is a homodimeric protein linked through cysteine residues in the hinge region of IgG Fc, resulting in a molecule similar to an IgG molecule without the CHI domains and light chains. Due to the structural homology, Fc fusion proteins exhibit in vivo pharmacokinetic profile comparable to that of human IgG with a similar isotype. This approach has been applied to several therapeutically important cytokines, such as IL-2 and IFN-α₂ₐ, and soluble receptors, such as TNF-Rc and IL-5-Rc (see, e.g., U.S. Pat. Nos. 5,349,053 and 6,224,867). Thus, to extend the circulating half-life of an MDL-1 fusion protein of the present invention and/or to increase its biological activity, it is desirable to make fusion proteins containing MDL-1 linked to the Fc portion of the human IgG protein as described herein.

“Fc-MDL-1 fusion protein” as used herein refers to a protein having at least an Fc portion fused directly or indirectly to an MDL-1 portion. Examples of such proteins include, but are not limited to the Fc-MDL-1 fusion protein encoded by: the amino acid sequence of SEQ ID NO: 1 (hFc-MDL-1); the nucleic acid sequence of SEQ ID NO: 2 (hFc-MDL-1); the amino acid sequence of SEQ ID NO: 3 (hFc-MDL-1); the nucleic acid sequence of SEQ ID NO: 4 (hFc-MDL-1); the amino acid sequence of SEQ ID NO: 5 (mFc-MDL-1); and the nucleic acid sequence of SEQ ID NO: 6 (mFc-MDL-1). More particularly, in one embodiment th Fc-MDL-1 fusion protein is a human Fc-MDL-1 fusion protein comprising a human Fc portion and a human MDL-1 portion (e.g., as encoded by: the amino acid sequence of SEQ ID NO: 1 (hFc-MDL-1); the nucleic acid sequence of SEQ ID NO: 2 (hFc-MDL-1); the amino acid sequence of SEQ ID NO: 3 (hFc-MDL-1); and the nucleic acid sequence of SEQ ID NO: 4 (hFc-MDL-1)). In another embodiment the Fc-MDL-1 fusion protein is a mouse Fc-MDL-1 fusion protein comprising a mouse Fc portion and a mouse MDL-1 portion (e.g., as encoded by the amino acid sequence of SEQ ID NO: 5 (mFc-MDL-1); the nucleic acid sequence of SEQ ID NO: 6 (mFc-MDL-1)). However, as described herein, chimeric and heterologous fusion proteins are encompassed by the compositions of the present invention.

Examples of suitable Fc portions for use in the fusion proteins of the present invention include, but are not limited to, the Fc portion encoded by: the amino acid sequence of SEQ ID NO: 7 (hFC); the nucleic acid sequence of SEQ ID NO: 8 (hFc); the amino acid sequence of SEQ ID NO: 9 (mFC); and the nucleic acid sequence of SEQ ID NO: 10 (mFc). Examples of suitable MDL-1 portions for use in the fusion proteins of the present invention include, but are not limited to, the MDL-1 extracellular domain (ECD) encoded by: the amino acid sequence of SEQ ID NO: 11 (hMDL-1 ECD); the nucleic acid sequence of SEQ ID NO: 12 (hMDL-1 ECD); the amino acid sequence of SEQ ID NO: 13 (mMDL-1 ECD); and the nucleic acid sequence of SEQ ID NO: 14 (mMDL-1 ECD).

In some embodiments of the present invention, the proteins having an Fc portion and an MDL-1 portion form homodimers. For example, in some embodiments, an Fc-MDL-1 fusion protein is a dimeric protein held together by one or more disulfide bonds, each protein chain containing an Fc portion and an MDL-1 portion. Further, the Fc-MDL-1 fusion proteins of the present invention can have any configuration allowing the MDL-1 portions to stably associate with Fc portions while maintaining MDL-1 activity. For example, such configurations include, but are not limited to, a single protein containing two Fc portions and two MDL-1 portions, a single protein containing two Fc portions and one MDL-1 portion, a heterodimeric protein including one protein containing an Fc portion and an MDL-1 portion and another protein containing an Fc portion, and other suitable configurations.

The MDL-1 portion can be directly or indirectly fused to the Fc portion in various configurations. For example, in some embodiments, the MDL-1 portion is indirectly fused to the Fc portion via a linker. In particular, in some embodiments, the Fc-MDL-1 fusion protein includes a linker between the Fc portion and the MDL-1 portion. In one embodiment, the MDL-1 portion is fused to the carboxyl-terminus (COOH-terminus or C-terminus) of the Fc portion through a linker. Examples of such Fc-MDL-1 proteins include, but are not limited to, those encoded by the: amino acid sequence of SEQ ID NO: 1 (hFc-MDL-1); nucleic acid sequence of SEQ ID NO: 2 (hFc-MDL-1); amino acid sequence of SEQ ID NO: 5 (mFc-MDL-1); and nucleic acid sequence of SEQ ID NO: 6 (mFc-MDL-1). In another embodiment, the MDL-1 portion is fused to the N-terminus of the Fc portion through a linker. Examples of such Fc-MDL-1 proteins include, but are not limited to, those encoded by the amino acid sequence of SEQ ID NO: 3 (hFc-MDL-1); and nucleic acid sequence of SEQ ID NO: 2 (hFc-MDL-4).

In other embodiments, the MDL-1 portion is directly fused to the Fc portion through a covalent bond. For example, in some embodiments, the MDL-1 portion is fused directly to the Fc portion at either its C-terminus or its N-terminus. In one embodiment, the C-terminus of the Fc portion is fused to the N-terminus of the MDL-1 portion. In this configuration, the Fc portion is towards the N-terminus of the Fc-MDL-1 fusion protein and the MDL-1 portion is towards the C-terminus. In another embodiment, the C-terminus of MDL-1 is fused to the N-terminus of the Fc portion. In this configuration, the MDL-1 portion is towards the N-terminus of the Fc-MDL-1 fusion protein and the Fc portion is towards the C-terminus.

As used herein, “MDL-1 portion” with reference to an MDL-1 fusion protein of the present invention refers to a portion of the fusion protein comprising a sequence of an MDL-1, including biologically-active fragments, analogs, variants, mutants or derivatives thereof.

The MDL-1 of the present invention encompasses wild-type or naturally-occurring MDL-1 from a mammalian species (e.g., human, non-human primate, or rodent), recombinant or synthetic MDL-1, and MDL-1-like molecules, including biologically-active fragments, analogs, variants, mutants, and derivatives of an MDL-1.

Examples of suitable MDL-1 sequences for use in the compositions of the present include, but are not limited to fragments, analogs, variants, mutants and derivatives of a sequence encoding a full-length MDL-1, e.g., the amino acid sequence of SEQ ID NO:15 (hMDL-1); the nucleic acid sequence of SEQ ID NO: 16 (hMDL-1); the amino acid sequence of SEQ ID NO: 17 (mMDL-1); and the nucleic acid sequence of SEQ ID NO: 18 (mMDL-1). Examples of suitable biologically active fragments for use in the compositions of the present invention include, but are not limited to, the MDL-1 extracellular domain encoded by: the amino acid sequence of SEQ ID NO: 11 (hMDL-1 ECD); the nucleic acid sequence of SEQ ID NO: 12 (hMDL-1 ECD); the amino acid sequence of SEQ ID NO: 13 (mMDL-1 ECD); and the nucleic acid sequence of SEQ ID NO: 14 (mMDL-1 ECD).

Biologically active variants of MDL-1 encompassed by the invention retain an MDL-1 activity. Examples of such activities include, but are not limited to, signal transduction, interacting or associating with an MDL-1 ligand or other binding partner (e.g., DAP12) or cellular component, modulating an inflammatory response or process, including myeloid cell activation. In some embodiments, the MDL-1 variant of the present invention retains at least about 25%, about 50%, about 75%, about 85%, about 90%, about 95%, about 98%, about 99%, or more of the biological or therapeutic activity of the reference MDL-1 (e.g., a naturally-occurring or wild-type MDL-1).

In particular embodiments, the compositions of the present invention comprise a full-length sequence or partial sequence (e.g., a fragment) of a wild-type or naturally-occurring MDL-1 of a mammalian species (e.g., human, non-human primate, or rodent), or MDL-1-like molecule as described herein, or as known in the art (e.g., see the mouse MDL-1 sequence and the human MDL1 sequence of Bakker, A. et al. (1999) 96:9792-9796). Examples of a full-length mammalian MDL-1 protein include, but are not limited to the protein encoded by: the amino acid sequence of SEQ.ID.NO: 15 (hMDL-1); the nucleic acid sequence of SEQ ID NO: 16 (hMDL-1); the amino acid sequence of SEQ ID NO: 17 (mMDL-1); the nucleic acid sequence of SEQ ID NO: 18 (mMDL-1); and the full-length human MDL-1 or mouse MDL-1 described in U.S. Pat. No. 6,416,973.

A partial MDL-1 sequence can be e.g., a fragment or truncation of a full-length sequence. More particularly, an MDL-1 fragment and truncation of the present invention comprise a sequence that is a partial sequence or portion of a full-length MDL-1 sequence, and retains a biological activity or function of the MDL-1. The fragments and truncations of the present invention can be produced by methods known in the art. For example, the MDL-1 fragments of the present invention can be produced by a deletion of amino- and/or carboxy-terminal residues or portion, and/or an internal deletion of residues or portion, of an MDL-1. Also, MDL-1 truncations of the present invention can be produced by the removal or deletion of amino- and/or carboxy-terminal residues of MDL-1. An MDL-1 of the present invention also encompasses hybrid or chimeric forms of MDL-1.

Chimeric or hybrid constructs may be made from combining similar functional domains from other proteins. For example, partner-binding or other segments may be “swapped” between different new fusion polypeptides or fragments. See, e.g., Cunningham, et al. (1989) Science 243:1330-1336; and O'Dowd, et al. (1988) J. Biol. Chem. 263:15985-15992. Thus, new chimeric polypeptides exhibiting new combinations of specificities can result from the functional linkage of partner-binding specificities and other functional domains.

In some embodiments, a biologically-active or functionally-active MDL-1-like molecule of the present invention has substantial amino acid sequence similarity or identity with a corresponding sequence of a wild-type, or naturally-occurring MDL-1 and has one or more of the functions or activities of a wild-type MDL-1 or naturally-occurring MDL-1. In some embodiments, a biologically- or functionally-active MDL-1-like molecule of the present invention has 85-100% sequence identity to a mammalian MDL-1 e.g., to the MDL-1 encoded by: the amino acid sequence of SEQ ID NO: 15 (hMDL-1); the nucleic acid sequence of SEQ ID NO: 16 (hMDL-1); the amino acid sequence of SEQ ID NO: 17 (mMDL-1); or the nucleic acid sequence of SEQ ID NO: 18 (mMDL-1). In some embodiments, the sequence identity is at least about 85%,-90%, or 90%-95%, and more particularly at least about 95%-99%. In particularly preferred embodiments, the sequence identity is at least about 99% or more

Thus, the MDL-1 of the present invention encompasses analogs of MDL-1. An analog of the present invention may be a form with structural modifications, or may be a wholly unrelated molecule which has a molecular shape which interacts with the appropriate surface binding determinants. The analogs can be agonists or antagonists, see, e.g., Goodman, et al. (eds. 1990) Goodman & Gilman's: The Pharmacological Bases of Therapeutics (8th ed.) Pergamon Press. In some embodiments, the analogs have an amino acid sequence with sufficient similarity to the amino acid sequence of a wild-type or naturally occurring MDL-1 to retain a biological function or activity of a wild-type or naturally occurring MDL-1. For example, an analog of MDL-1 can contain one or more amino acid changes in the amino acid sequence of wild-type MDL-1, and retain the ability to interact with an MDL-1 ligand, and/or DAP12, and further, to signal transduce. Examples of such amino acid changes include, but are not limited to, additions, deletions or substitutions of amino acid residues. The MDL-1 of the present invention also encompasses mutant proteins that exhibit a greater, lesser, or altered biological activity or function than a wild-type or naturally occurring MDL-1. For example, an MDL-1 protein of the present invention can be mutated or constructed in such a manner as described herein to be a competitive inhibitor and, thereby, have an altered or different MDL-1 activity or function as compared to a wild-type or naturally-occurring MDL-1.

Using methods known in the art, amino acid modifications can be introduced into an MDL-1 or MDL-1 portion of the present invention to alter a function or activity of the MDL-1, e.g., to alter the binding affinity; protein stability; conformation; pharmacokinetic properties; synthesis or expression; or to provide other advantageous features. Methods for introducing such mutations are known in the art and include, but are not limited to site-directed, random, and semi-random mutagenesis.

The present invention also includes, but is not limited to, variants or biological functional equivalents of MDL-1. Such substitutions are those that substitute a given amino acid in a protein by another amino acid of like characteristics. Amino acid sequence identity can be determined by optimizing residue matches. Conservative substitutions typically include substitutions within the following groups: glycine, alanine; valine, isoleucine, leucine; aspartic acid, glutamic acid; asparagine, glutamine; serine, threonine; lysine, arginine; and phenylalanine, tyrosine. Similar amino acid sequences are intended to include natural allelic variations in each respective protein sequence.

Homologous nucleic acid sequences, when compared, exhibit significant sequence similarity. The standards for homology in nucleic acids are either measures for homology generally used in the art by sequence comparison or based upon hybridization conditions. The hybridization conditions are described in greater detail below.

Homologous proteins or peptides of the present invention encompass those having 85-100% sequence identity. In some embodiments, the sequence identity is at least about 85%,-90%, or 90%-95%, and more particularly at least about 95%-99%. In particularly preferred embodiments, the sequence identity is at least about 99% or more. With reference to methods for determining sequence identity, see e.g., Needleham, et al. (1970) J. Mol. Biol. 48:443-453; Sankoff, et al. (1983) Chapter One in Time Wars, String Edits, and Macromolecules: The Theory and Practice of Sequence Comparison Addison-Wesley, Reading, Mass.; and software packages from IntelliGenetics, Mountain View, Calif.; and the University of Wisconsin Genetics Computer Group, Madison, Wis.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optical alignment of sequences for comparison can be conducted, e.g., by the local homology algorithm of Smith and Waterman (1981) Adv. Appl. Math. 2:482, by the homology alignment algorithm of Needleman and Wunsch (1970) J. Mol. Biol. 48:443, by the search for similarity method of Pearson and Lipman (1988) Proc. Nat'l Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., supra).

One example of a useful algorithm is PILEUP. PILEUP creates a multiple sequence alignment from a group of related sequences using progressive, pairwise alignments to show relationship and percent sequence identity. The algorithm also plots a tree or dendogram showing the clustering relationships used to create the alignment. PILEUP uses a simplification of the progressive alignment method of Feng and Doolittle (1987) J. Mol. Evol. 35:351-360. The method used is similar to the method described by Higgins and Sharp (1989) CABIOS 5:151-153. The program can align up to 300 sequences, each of a maximum length of 5,000 nucleotides or amino acids. The multiple alignment procedure begins with the pairwise alignment of the two most similar sequences, producing a cluster of two aligned sequences. This cluster can then aligned to the next most related sequence or cluster of aligned sequences. Two clusters of sequences are aligned by a simple extension of the pairwise alignment of two individual sequences. The final alignment can be achieved by a series of progressive, pairwise alignments. The program is run by designating specific sequences and their amino acid or nucleotide coordinates for regions of sequence comparison and by designating the program parameters. For example, a reference sequence can be compared to other test sequences to determine the percent sequence identity relationship using the following parameters: default gap weight (3.00), default gap length weight (0.10), and weighted end gaps.

Another example of algorithm that is suitable for determining percent sequence identity and sequence similarity is the BLAST algorithm, which is described Altschul, et al. (1990) J. Mol. Biol. 215:403-410. Software for performing BLAST analyses is publicly available through the National Center for Biotechnology Information (at the ncbi.nlm.nih.gov Web link). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul, et al., supra). These initial neighborhood word hits act as seeds for initiating searches to find longer HSPs containing them. The word hits are then extended in both directions along each sequence for as far as the cumulative alignment score can be increased. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLAST program uses as defaults a wordlength (W) of 11, the BLOSUM62 scoring matrix (see Henikoff and Henikoff (1989) Proc. Nat'l Acad. Sci. USA 89:10915) alignments (B) of 50, expectation (E) of 10, M=5, N=4, and a comparison of both strands.

In addition to calculating percent sequence identity, the BLAST algorithm also performs a statistical analysis of the similarity between two sequences (see, e.g., Karlin and Altschul (1993) Proc. Nat'l Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

A further indication that two nucleic acid sequences of polypeptides are substantially identical is that the polypeptide encoded by the first nucleic acid is immunologically cross reactive with the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules hybridize to each other under stringent conditions.

Stringent conditions, in referring to identity in the hybridization context, can be stringent combined conditions of salt, temperature, organic solvents, and other parameters typically controlled in hybridization reactions. Stringent temperature conditions will usually include temperatures in excess of about 30° C., more usually in excess of about 37° C., typically in excess of about 45° C., more typically in excess of about 55° C., preferably in excess of about 65° C., and more preferably in excess of about 70° C. Stringent salt conditions will ordinarily be less than about 500 mM, usually less than about 350 mM, more usually less than about 200 mM, typically less than about 150 mM, preferably less than about 100 mM, and more preferably less than about 50 mM. However, the combination of parameters is much more important than the measure of any single parameter (see, e.g., Wetmur and Davidson (1968) J. Mol. Biol. 31:349-370). Hybridization under stringent conditions should give a background of at least 2-fold over background, preferably at least 3-5 or more.

Further, MDL-1 variants may comprise a full-length or partial (i.e., fragment) MDL-1 sequence, in which several, 5 to 10, 1 to 5, 1 to 3, 2, 1 or no amino acid residues are substituted, deleted or added, in any combination. Such variants of MDL-1 encompass biological functional equivalents, such as those retaining similar or altered (e.g., decreased or increased) MDL-1 ligand or DAP12 binding affinity or specificity.

Derivatives of MDL-1 include amino acid sequence mutants, glycosylation variants, and covalent or aggregate conjugates with other chemical moieties. Covalent derivatives can be prepared by linkage of functionalities to groups which are found in the MDL-1 amino acid side chains or at the N- or C-termini, by means which are well known in the art. These derivatives can include, without limitation, aliphatic esters or amides of the carboxyl terminus, or of residues containing carboxyl side chains, O-acyl derivatives of hydroxyl group-containing residues, and N-acyl derivatives of the amino terminal amino acid or amino-group containing residues, e.g., lysine or arginine. Acyl groups are selected from the group of alkyl-moieties including C3 to C18 normal alkyl, thereby forming alkanoyl aroyl species.

In particular, glycosylation alterations are encompassed, e.g., made by modifying the glycosylation patterns of a protein during its synthesis and processing, or in further processing steps. While there are no natural N-linked sites on the protein, there may be O-linked sites, or variants with such sites may be produced. Particularly preferred means for accomplishing this are by exposing the polypeptide to glycosylating enzymes derived from cells which normally provide such processing, e.g., human glycosylation enzymes. Deglycosylation enzymes are also contemplated. Also encompassed are versions of the same primary amino acid sequence which have other minor modifications, including phosphorylated amino acid residues, e.g., phosphotyrosine, phosphoserine, or phosphothreonine.

Derivatives can be e.g., covalent conjugates of MDL-1 or fragments thereof. These derivatives can be synthesized in recombinant culture such as N- or C-terminal fusions or by the use of agents known in the art for their usefulness in cross-linking proteins through reactive side groups. Preferred derivatization sites with cross-linking agents are at free amino groups, carbohydrate moieties, and cysteine residues.

Fusion proteins may be constructed to exhibit a combination of properties or activities of the derivative proteins. An example of a heterologous fusion protein is a reporter protein, e.g., luciferase fused to an MDL-1 or fragment thereof. Methods of constructing fusion proteins are known (see, e.g., Dull, et al., U.S. Pat. No. 4,859,609). Other gene fusion partners include bacterial beta-galactosidase, trpE, Protein A, beta-lactamase, alpha amylase, alcohol dehydrogenase, and yeast alpha mating factor. See, e.g., Godowski, et al. (1988) Science 241:812-816. The phosphoramidite method described by Beaucage and Carruthers (1981) Tetra. Lefts. 22:1859-1862, can be used to produce suitable synthetic DNA fragments.

The present invention also contemplates the use of derivatives that involve covalent or aggregative association with chemical moieties. These derivatives generally fall into three classes: (1) salts, (2) side chain and terminal residue covalent modifications, and (3) adsorption complexes, for example with cell membranes. Such covalent or aggregative derivatives are useful as immunogens, as reagents in immunoassays, or in purification methods such as for affinity purification of binding partners. For example, an MDL-1 of the present invention can be immobilized by covalent bonding to a solid support such as cyanogen bromide-activated Sepharose, by methods which are well known in the art, or adsorbed onto polyolefin surfaces, with or without glutaraldehyde cross-linking, for use in the assay or purification of anti-MDL-1 antibodies or its binding partners. The MDL-1 can also be labeled with a detectable group, for example radioiodinated onto a tyrosine, e.g., incorporated into the natural sequence, by the chloramine T procedure, covalently bound to rare earth chelates, or conjugated to another fluorescent moiety for use in diagnostic assays. A solubilized MDL-1 of the present invention can be used as an immunogen for the production of antisera or antibodies specific for the antigen or many fragments thereof. The purified antigens can be used to screen monoclonal antibodies or antigen-binding fragments prepared by immunization with various forms of impure preparations containing the protein.

The present invention contemplates the isolation of additional closely related variants. It is highly likely that allelic variations exist in different individuals exhibiting, e.g., better than 90-97% identity to the embodiment described herein. Dissection of the critical structural elements which effect the various differentiation functions provided by receptor binding is possible using standard techniques of modern molecular biology, particularly in comparing members of the related class. See, e.g., the homolog-scanning mutagenesis technique described in Cunningham, et al. (1989) Science 243:1339-1336; and approaches used in O'Dowd, et al. (1988) J. Biol. Chem. 263:15985-15992; and Lechleiter, et al. (1990) EMBO J. 9:4381-4390.

In particular, receptor partner binding segments can be substituted between species variants to determine what structural features are important in both binding affinity and specificity. An array of different, e.g., MDL-1 variants, can be used to screen for partners exhibiting combined properties of interaction with different species variants. Intracellular functions would probably involve segments of the antigen which are normally accessible to the cytosol. The specific segments of interaction of MDL-1 with other intracellular components may be identified by mutagenesis or direct biochemical means, e.g., cross-linking, affinity, or genetic methods. Structural analysis by crystallographic or other physical methods can also be applied. Further investigation of the mechanism of signal transduction can include study of associated components which may be isolatable by known affinity methods,

The present invention also encompasses structurally similar compounds formulated to mimic the key portions of the proteins of the present invention. Such compounds, e.g., peptidomimetics, may be used in the same manner as, and as functional equivalents of, the proteins or peptides of the present invention. Mimetics that mimic elements of protein secondary and tertiary structure are known the art (e.g., see Johnson et al., (1993). An underlying rationale concerning the use of peptide mimetics is that the peptide backbone of proteins orient amino acid side chains in such a way as to facilitate molecular interactions, such as those of antibody and/or antigen. A peptide mimetic can thus be designed to permit molecular interactions similar to the natural molecule. Some successful applications of the peptide mimetic concept have focused on mimetics of β-turns within proteins, which are known to be highly antigenic. A β-turn structure within a protein can be predicted by computer-based algorithms, and once the component amino acids of the turn are determined, mimetics can be constructed to achieve a similar spatial orientation of the essential elements of the amino acid side chains.

Other approaches include the use of small, multidisulfide-containing proteins as structural templates for producing biologically active conformations that mimic the binding sites of large proteins (Vita et al., 1998). A structural motif that is reportedly evolutionarily conserved in certain toxins is 30-40 amino acids, stable, and highly permissive for mutation. The motif is reportedly composed of a beta sheet and an alpha helix bridged in the interior core by three disulfides. Further, beta II turns have been reportedly mimicked successfully using cyclic L-pentapeptides and those with D-amino acids. Weisshoff et al., (1999). Further, Johannesson et al., (1999) reported bicyclic tripeptides with reverse turn inducing properties.

Methods for generating specific structures are known in the art. For example, alpha-helix mimetics are reported in U.S. Pat. Nos. 5,446,128; 5,710,245; 5,840,833; and 5,859,184. Theses structures reportedly render the peptide or protein more thermally stable, increase resistance to proteolytic degradation. Further, methods for generating conformationally restricted beta turns and beta bulges are known (see e.g., U.S. Pat. Nos. 5,440,013; 5,618,914; and 5,670,155). Beta-turns permit changed side substituents without having changes in corresponding backbone conformation, and have appropriate termini for incorporation into peptides by standard synthesis procedures. Other types of mimetic turns include reverse and gamma turns. Reverse turn mimetics are disclosed in U.S. Pat. Nos. 5,475,085 and 5,929,237, and gamma turn mimetics are described in U.S. Pat. Nos. 5,672,681 and 5,674,976.

The protein compositions of the present invention may also have amino acid residues which have been chemically modified by phosphorylation, sulfonation, biotinylation, or the addition or removal of other moieties, particularly those which have molecular shapes similar to phosphate groups. In some embodiments, the modifications can be useful labeling reagents, or serve as purification targets, e.g., affinity reagents.

In particular, the term “antibodies” also encompasses antigen binding fragments of natural antibodies. A purified MDL-1 can also be used as a reagent to detect antibodies generated in response to the presence of elevated levels of MDL-1 or cell fragments containing the MDL-1, both of which may be diagnostic of an abnormal or specific physiological or disease condition. Additionally, MDL-1 fragments may also serve as immunogens to produce the antibodies of the present invention, as described immediately below. In particular, the present invention contemplates antibodies having binding affinity to or being raised against specific fragments which are predicted to lie outside of the lipid bilayer, either extracellular or intracellular domains. Additionally, various constructs may be produced from fusion of a membrane associating segment to the otherwise extracellular exposed portion of the molecule. Other antigenic complexes may be used, including complexes of MDL-1 with a receptor partner or ligand.

The compositions of the present invention also include polyclonal or monoclonal antibodies directed against MDL-1, and fusion proteins comprising an antibody portion and an MDL-1 portion (e.g., and Fc-MDL-1 fusion protein). As used herein with reference to an Fc-MDL-1 fusion protein of the present invention, “Fc portion”, encompasses domains derived from the constant region of a mammalian immunoglobulin (e.g. a human, non-human primate, or rodent immunoglobulin), including a fragment, analog, variant, mutant or derivative of the constant region. Suitable immunoglobulins (IgG) include, but are not limited to, IgG1, IgG2, IgG3, IgG4, and other classes. The constant region of an immunoglobulin is defined as a naturally-occurring or synthetically-produced polypeptide homologous to the immunoglobulin C-terminal region, and can include a CH1 domain, a hinge, a CH2 domain, a CH3 domain, or a CH4 domain, separately or in combination. According to Paul, (1999) Fundamental Immunology 4.sup.th Ed., Lippincott-Raven, CH1 domain includes amino acids 118-215; hinge region includes amino acids 216-230; CH2 domain includes amino acids 231-340; and CH3 domain includes amino acids 341-447 (the amino acid positions are based on IgG1 sequence). The hinge region joins the CH1 domain to the CH2 and CH3 domains.

In some embodiments, the Fc portion of the present invention comprises at least a CH2 domain. For example, in one embodiment the Fc portion includes hinge-CH2-CH3. In another embodiment, the Fc portion includes all or a portion of the hinge region, the CH2 domain and/or the CH3 domain.

The constant region of an immunoglobulin is responsible for many important antibody functions including Fc receptor (FcR) binding and complement fixation. There are five major classes of heavy chain constant region, classified as IgA, IgG, IgD, IgE, IgM, each with characteristic effector functions designated by isotype. For example, IgG is separated into four gamma subclasses: IgG1, IgG2, IgG3, and IgG4.

IgG molecules interact with multiple classes of cellular receptors including three classes of Fc.gamma. receptors (Fc.gamma.R) specific for the IgG class of antibody, namely Fc.gamma.RI, Fc.gamma.RII, and Fc.gamma.RIII. The important sequences for the binding of IgG to the Fc.gamma.R receptors have been rMDL-1rted to be located in the CH2 and CH3 domains. The serum half-life of an antibody is influenced by the ability of that antibody to bind to an Fc receptor (FcR). Similarly, the serum half-life of immunoglobulin fusion proteins is also influenced by the ability to bind to such receptors (Gillies S D et al., (1999) Cancer Res. 59:2159-66). Compared to those of IgG1, CH2 and CH3 domains of IgG2 and IgG4 have biochemically undetectable or reduced binding affinity to Fc receptors. It has been rMDL-1rted that immunoglobulin fusion proteins containing CH2 and CH3 domains of IgG2 or IgG4 had longer serum half-lives compared to the corresponding fusion proteins containing CH2 and CH3 domains of IgG1 (U.S. Pat. No. 5,541,087; Lo et al., (1998) Protein Engineering, 11:495-500). Accordingly, preferred CH2 and CH3 domains for the present invention are derived from an antibody isotype with reduced receptor binding affinity and effector functions, such as, for example, IgG2 or IgG4. More preferred CH2 and CH3 domains are derived from IgG2.

The hinge region is normally located C-terminal to the CH1 domain of the heavy chain constant region. In the IgG isotypes, disulfide bonds typically occur within this hinge region, permitting the final tetrameric molecule to form. This region is dominated by prolines, serines and threonines. When included in the present invention, the hinge region is typically at least homologous to the naturally-occurring immunoglobulin region that includes the cysteine residues to form disulfide bonds linking the two Fc moieties.

Representative sequences of hinge regions for human and mouse immunoglobulins can be found in Borrebaeck, C. A. K., ed., (1992) ANTIBODY ENGINEERING, A PRACTICAL GUIDE, W.H. Freeman and Co. Suitable hinge regions for the present invention can be derived from IgG1, IgG2, IgG3, IgG4, and other immunoglobulin classes. The IgG1 hinge region has three cysteines, two of which are involved in disulfide bonds between the two heavy chains of the immunoglobulin. These same cysteines permit efficient and consistent disulfide bonding formation between Fc portions. Therefore, a preferred hinge region of the present invention is derived from IgG1, more preferably from human IgG1. In some embodiments, the first cysteine within the human IgG1 hinge region is mutated to another amino acid, preferably serine. The IgG2 isotype hinge region has four disulfide bonds that tend to promote oligomerization and possibly incorrect disulfide bonding during secretion in recombinant systems. A suitable hinge region can be derived from an IgG2 hinge; the first two cysteines are each preferably mutated to another amino acid. The hinge region of IgG4 is known to form interchain disulfide bonds inefficiently. However, a suitable hinge region for the present invention can be derived from the IgG4 hinge region, preferably containing a mutation that enhances correct formation of disulfide bonds between heavy chain-derived moieties (Angal S, et al. (1993) Mol. Immunol., 30:105-8).

In accordance with the present invention, the Fc portion can contain CH2 and/or CH3 domains and a hinge region that are derived from different antibody isotypes, i.e., a hybrid Fc portion. For example, in one embodiment, the Fc portion contains CH2 and/or CH3 domains derived from IgG2 or IgG4 and a mutant hinge region derived from IgG1. Alternatively, a mutant hinge region from another IgG subclass is used in a hybrid Fc portion. For example, a mutant form of the IgG4 hinge that allows efficient disulfide bonding between the two heavy chains can be used. A mutant hinge can also be derived from an IgG2 hinge in which the first two cysteines are each mutated to another amino acid. Such hybrid Fc portions facilitate high-level expression and improve the correct assembly of the Fc-MDL-1 fusion proteins. Assembly of such hybrid Fc portions has been described in U.S. Patent Publication No. 20030044423 (i.e., U.S. application Ser. No. 10/093,958), the disclosure of which is hereby incorporated by reference.

In some embodiments, the Fc portion contains amino acid modifications that generally extend the serum half-life of an Fc fusion protein. Such amino acid modifications include mutations substantially decreasing or eliminating Fc receptor binding or complement fixing activity. For example, the glycosylation site within the Fc portion of an immunoglobulin heavy chain can be removed. In IgG1, the glycosylation site is Asn297. In other immunoglobulin isotypes, the glycosylation site corresponds to Asn297 of IgG1. For example, in IgG2 and IgG4, the glycosylation site is the asparagine within the amino acid sequence Gln-Phe-Asn-Ser. Accordingly, a mutation of Asn297 of IgG1 removes the glycosylation site in an Fc portion derived from IgG1. In one embodiment, Asn297 is replaced with Gln. Similarly, in IgG2 or IgG4, a mutation of asparagine within the amino acid sequence Gln-Phe-Asn-Ser removes the glycosylation site in an Fc portion derived from IgG2 or IgG4 heavy chain. In one embodiment, the asparagine is replaced with a glutamine. In other embodiments, the phenylalanine within the amino acid sequence Gln-Phe-Asn-Ser is further mutated to eliminate a potential non-self T-cell epitope resulting from asparagine mutation. For example, the amino acid sequence Gln-Phe-Asn-Ser within an IgG2 or IgG4 heavy chain can be replaced with a Gln-Ala-Gln-Ser amino acid sequence.

The alteration of amino acids near the junction of the Fc portion and the non-Fc portion reportedly can dramatically increase the serum half-life of the Fc fusion protein (PCT publication WO 01/58957, the disclosure of which is hereby incorporated by reference). Accordingly, the junction region of an Fc-MDL-1 fusion protein of the present invention can contain alterations that, relative to the naturally-occurring sequences of an immunoglobulin heavy chain and MDL-1, preferably lie within about 10 amino acids of the junction point. These amino acid changes can cause an increase in hydrophobicity by, for example, changing the C-terminal lysine of the Fc portion to a hydrophobic amino acid such as alanine or leucine.

In other embodiments, the Fc portion contains amino acid alterations of the Leu-Ser-Leu-Ser segment near the C-terminus of the Fc portion of an immunoglobulin heavy chain. The amino acid substitutions of the Leu-Ser-Leu-Ser segment eliminate potential junctional T-cell epitopes. In one embodiment, the Leu-Ser-Leu-Ser amino acid sequence near the C-terminus of the Fc portion is replaced with an Ala-Thr-Ala-Thr amino acid sequence. In other embodiments, the amino acids within the Leu-Ser-Leu-Ser segment are replaced with other amino acids such as glycine or proline. Detailed methods of generating amino acid substitutions of the Leu-Ser-Leu-Ser segment near the C-terminus of an IgG1, IgG2, IgG3, IgG4, or other immunoglobulin class molecule are known (see, e.g., U.S. patent application Ser. No. 10/112,582), the disclosure of which is hereby incorporated by reference.

In some embodiments, the fusion proteins of the present invention have two or more amino acid sequences or portions fused together via a linker. For example, the Fc-MDL-1 fusion proteins of the present invention can include a linker between an Fc portion and an MDL-1 portion. A fusion protein with a linker may have improved properties, such as increased biological activity. In some embodiment, the linker contains between 1 and 25 amino acids (e.g., between 5 and 25 or between 10 and 20 amino acids). In one embodiment, the linker is designed to include no protease cleavage site. In another embodiment, the linker contains an N-linked or an O-linked glycosylation site to sterically inhibit proteolysis. Accordingly, in one embodiment, the linker contains an Asn-Ala-Thr amino acid sequence. Additional suitable linkers are disclosed in Robinson et al., (1998), Proc. Natl. Acad. Sci. USA; 95, 5929; and U.S. application Ser. No. 09/708,506.

Pharmaceutical composition of the present invention also include prodrug derivatives. As used herein “prodrug” refers to a pharmacologically inactive (or partially inactive) derivative of a parent molecule that requires biotransformation, either spontaneous or enzymatic, within an organism (e.g., a patient) to release or activate an active component. Prodrugs are variants or derivatives of the pharmaceutical compositions of the present invention and more particularly, are enzymatically cleavable under metabolic conditions. A prodrugs of the present invention is metabolized or enzymatically cleaved and, thereby, results in a composition of the present invention that modulates MDL-1 activity in a cell of a patient. In some embodiments, a prodrug of the present invention can undergo more than one biotransformation step (e.g., enzymatic modification or processing e.g., enzymatic cleavage) in vivo in order release or activate the active component (i.e., a composition that modulates MDL-1 activity). The prodrugs of the present invention can confer advantages of solubility, tissue compatibility, or delayed release in a mammalian patient or subject. Methods for making prodrugs are known in the art (see, e.g., Bundgard, (1985) Design of Prodrugs, pp. 7-9, 21-24, Elsevier, Amsterdam; Silverman, (1992) The Organic Chemistry of Drug Design and Drug Action, pp. 352-401, Academic Press, San Diego, Calif.). Moreover, the prodrug derivatives according to this invention can be combined with other features to enhance bioavailability.

Methods of Treatment

The present invention provides methods for treatment of inflammatory disease in a patient in need of treatment thereof, by administering a composition of the present invention that modulates MDL-1 activity in a cell of the patient (e.g., a human, non-human primate, or rodent). In particular, the present invention provides methods of modulating an immune process or response mediated by MDL-1, for treatment of inflammatory disease, e.g., MS, IBD, and arthritis.

Reportedly, immune activation is a major contributory factor to the pathogenesis of inflammatory diseases such as MS, IBD, and arthritis. Immune abnormality in inflammatory diseases is reportedly associated with several changes in the immune system, including massive infiltration of neutrophils, lymphocytes, and macrophages (e.g., Wen and Fiocchi (2004) Clin. Dev. Immunol. 11:195-204). Further, the production of pro-inflammatory chemokines and cytokines, such as MCP-1, IL-2, IFN-γ, and TNF-α by infiltrating inflammatory cells, reportedly plays a pivotal role in the pathogenesis of inflammatory diseases. For example, treatment with the anti-TNF-α monoclonal antibody, Infliximab, reportedly results in clinical improvement and induced remission in patients with moderate-to-severe luminal and fistular Crohn's disease refractory (e.g., Sandborn (2005) Rev. Gastroenterol. Disord. 5:10-18).

In some embodiments, the present invention provides methods of treating inflammatory disease by decreasing MDL-1 activity or function, including signal transduction. For example in one embodiment a competitive inhibitor or decoy protein competes with cellular MDL-1 for binding of an MDL-1 ligand, thereby decreasing MDL-1 activity or function. In one embodiment, the competitive inhibitor or decoy protein is an Fc-MDl-1 fusion protein e.g., the Fc-MDL-1 fusion protein encoded by the nucleic acid sequence of SEQ ID NO: (hMDL-1), amino acid sequence of SEQ ID NO: (hMDL-1), nucleic acid sequence of SEQ ID NO: (mMDL-1), or amino acid sequence of SEQ ID NO: (mMDL-1). In another embodiment, an antagonist binds to or interacts with MDL-1 directly or indirectly (e.g., via an MDL-1 ligand), thereby, decreasing MDL-1 activity or function. In one embodiment, the antagonist is an anti-MDL-1 polyclonal or monoclonal antibody.

In some embodiments, the present invention provides methods of treating inflammatory disease by decreasing myeloid cell activation via the modulation (e.g., a decrease) in the activity or formation of the DAP12/MDL-1 complex. For example, in one embodiment, a competitive inhibitor or antagonist of MDL-1 is used to modulate the activity or formation of the DAP12/MDL-1 complex to decrease myeloid cell activation and, thereby, decrease inflammation. In one embodiment, the competitive inhibitor is a fusion protein and, more particularly, is an Fc-MDL-1 fusion protein e.g., the Fc-MDL-1 fusion protein encoded by the nucleic acid sequence of SEQ ID NO: (hMDL-1), amino acid sequence of SEQ ID NO: (hMDL-1), nucleic acid sequence of SEQ ID NO: (mMDL-1), or amino acid sequence of SEQ ID NO: (mMDL-1).

The compositions of the present invention can be administered to a mammalian patient (e.g., a human, non-human primate, or rodent) in an amount effective to result in the modulation of the immune process or response for treatment of an inflammatory disease. In particular, the compositions of the present invention can be administered to a mammalian patient in an amount effective to result in the modulation of an MDL-1 activity, including e.g., MDL-1 signal transduction and, more particularly, myeloid cell activation mediated by MDL-1.

Myeloid cell activation comprises activation of monocytes, macrophages and/or neutrophils. The activation can be specific for monocytes/macrophages or specific for neutrophils. In addition, myeloid cell activation may include a combination of activated monocytes, macrophages and neutrophils. Therefore, the compositions and methods of the present invention contemplate modulating the activation of monocytes, macrophages and/or neutrophils, and any stage of differentiation of these cells and, thereby, modulate an immune process or response for treatment of an inflammatory disease.

In some embodiments, the competitive inhibitor is a soluble MDL-1 receptor or functional equivalent that competes for a ligand of MDL-1 and/or decreases myeloid cell activation by down regulating the activity of the DAP12/MDL-1 complex or DAP12 pathway. Soluble forms of MDL-1 can down regulate or decrease the ligand mediated activation of cells and/or MDL-1 signal transduction, e.g., by binding to an MDL-1 ligand and making the ligand unavailable to MDL-1.

Further, in some embodiments, the present methods of modulating the inflammatory process in a patient or subject suffering from an inflammatory disease comprises the step of modulating the activity or formation of the DAP12/MDL-1 complex by modulating the interaction or association (e.g., binding) of a ligand with MDL-1.

In another embodiment, the competitive inhibitor is a soluble form of the cell-surface MDL-1 receptor. Deletion mutagenesis can be performed to confirm the specific region that is necessary to bind a ligand of MDL-1 and mediate the cellular response and/or activity or formation of the DAP12/MDL-1 complex. Further, amino acid substitution may be utilized to alter the transmembrane domain of MDL-1 to achieve a soluble form. Amino acid substitutions may also be used to increase the binding affinity of a ligand or competitive inhibitor of MDL-1, and thereby, modulate the activity or function of MDL-1.

In one embodiment, the present invention provides methods of myeloid cell-mediated immunotherapy comprising the step of administering to an mammalian patient (e.g., a human, non-human primate, or rodent) an anti-MDL-1 antibody or other composition that interacts with (e.g., binds to or associates with) MDL-1, to decrease cellular levels of MDL-1 or MDL-1 activity or function. Examples of such compositions include, but are not limited to, a polyclonal or monoclonal antibody that binds to MDL-1.

The present invention further provides methods of treating an inflammatory disease having a step of administering a pharmaceutical composition of the present invention comprising a pharmaceutical carrier admixed with a composition of the present invention. In some embodiments, administration is via a parenteral or alimentary route, topical, inhalation, or intraarticular.

Nucleic acid and chemical compositions for modulating MDL-1 activity and function are within the scope of and contemplated by the present application. For example, small molecule compounds, or nucleic acid compositions such as MDL-1 antisense RNA, ribozymes may be used (e.g., as antagonists or agonists) to modulate MDl-1 activity or function in a cell, in vivo or in vitro. Antisense RNA and RNA with ribozyme activity can decrease or diminish the expression of polynucleotides and consequently, production of an encoded protein product. Methods for preparing antisense RNA and ribozymes are known in the art. Further, ribozymes may be produced which act as endoribonucleases and catalyze the cleavage of RNA molecules with selected sequences resulting in reduction of the protein products. Thus, one of skilled in the art may use a variety of compounds to modulate the expression of MDL-1 for the treatment of inflammatory disease.

Examples of an inflammatory disease suitable for treatment using the compositions and methods of the present invention include, but are not limited to, MS, IBD, and arthritis.

IBD is characterized by chronic and relapsing bowel inflammation. Crohn's disease (CD) and ulcerative colitis are the two main forms of IBD. There are ˜1-2 million IBD patients in the USA, with ˜ half having CD. CD is associated with changes in the immune system, including massive mucosal infiltration of neutrophils, lymphocytes, and macrophages. The production by these infiltrating cells of pro-inflammatory chemokines and cytokines, such as IL-2, IFN-γ, and TNF-α, plays a pivotal role in CD pathogenesis. Anti-TNF therapies are used to treat subsets of patients with Crohn's disease, but there is high medical need for innovative drugs with a superior side effect profile to anti-TNF therapies and to treat patients not responding to anti-TNF therapy.

Inflammatory bowel diseases (IBD), which are comprised of Crohn's disease and ulcerative colitis, are characterized, e.g., by the clinical course of succession of relapses and remissions, and by chronically relapsing inflammation of the bowel. Current statistics indicate that there are 1-2 million Americans suffering from IBD with half of them diagnosed as having Crohn's disease (e.g., Head and Jurenka (2004) Alternative Med. Rev. 9:360-401). IBD causes much personal suffering and disablement for patients and represent a substantial economic burden on healthcare resources.

Targeted Diagnostic and Therapeutic Compositions

The compositions of the present invention can be used to target (i.e., directly or indirectly bind to, interact with, and/or otherwise associate with or is directed to) MDL-1 and/or an MDL-1 binding partner (e.g., MDL-1 ligand or DAP12) in a cell, tissue, or area of a subject, and therefore are useful for targeted therapeutic and diagnostic applications. In particular, the compositions of the present invention can be used to target a specific cell, tissue, or area of a subject where MDL-1 and/or MDL-1 binding partner is present for a diagnostic and/or therapeutic purpose. For example, the present invention provides diagnostic and therapeutic compositions that target (i.e., directly or indirectly bind to, interact with, and/or otherwise associate with or is directed to) MDL-1 and/or an MDL-1 binding partner (e.g., MDL-1 ligand or DAP12) in a cell, tissue, or area of a subject, and methods of using such compositions to image and/or treat an inflammatory disease state or condition. Thus, such compositions and methods can be used to treat, diagnose, and/or monitor the progression or treatment of, an inflammatory disease (e.g., MS, IBD, or arthritis). More particularly, the present invention provides diagnostic and therapeutic imaging agents and methods of generating an image of a subject comprising administering an imaging agent of the present invention (e.g., that targets MDL-1 and/or an MDL-1 binding partner) to the subject, and generating an image of a targeted cell, tissue, or area of a subject using known imaging modalities, e.g. by X-ray, MR, ultrasound, scintigraphic, PET, SPECT, electrical impedance, light, or magnetometric imaging.

In embodiments of the invention, the imaging agent comprises: a reporter moiety detectable in in vivo imaging in a cell or body of a mammalian subject (e.g., human, non-human primate, or rodent); a linker moiety or bond; and a targeting moiety (that directly or indirectly binds to, interacts with, and/or otherwise associates with or is directed to) MDL-1 and/or an MDL-1 binding partner (e.g., MDL-1 ligand or DAP12). Examples of targeting moieties of the present invention include, but are not limited to, an anti-MDL-1 antibody, MDL-1 fusion protein, and more particularly, an MDL-1 competitor or decoy protein that targets and MDL-1 binding partner. Reporter moieties and linker moieties suitable for use in such imaging agents are known in the art and known methods can be used to attach the linker moieties and reporter moieties to the targeting moiety.

The linker component of the imaging agent of the present invention is at its simplest a bond between a targeting and reporter moiety. More generally, the linker can provide a mono- or multi-molecular skeleton, covalently or non-covalently linking one or more targeting moieties to one or more reporters (e.g., a linear, cyclic, branched or reticulate molecular skeleton, or a molecular aggregate, with in-built or pendant groups which bind covalently or non-covalently, e.g. coordinatively, with the targeting and reporter moieties or which encapsulate, entrap or anchor such moieties). Thus, linking of a reporter moiety to the targeting moiety may be achieved by covalent or non-covalent means, involving interaction with one or more functional groups located on the reporter moiety and/or targeting moiety. Examples of chemically reactive functional groups which may be employed for this purpose include, but are not limited to, amino, hydroxyl, sulfhydryl, carboxyl, and carbonyl groups, as well as carbohydrate groups, vicinal diols, thioethers, 2-aminoalcohols, 2-aminothiols, guanidinyl, imidazolyl and phenolic groups. The target-reporter moiety coupling may also be effected using enzymes as zero-length crosslinking agents. For example, transglutaminase, peroxidase and xanthine oxidase have been used to produce crosslinked products. Reverse proteolysis may also be used for crosslinking through amide bond formation.

In some embodiments, the linking agent comprises two or more reactive moieties connected by a spacer element. In one embodiment, the presence of such a spacer permits bifunctional linkers to react with specific functional groups within a molecule or between two different molecules, resulting in a bond between these two components and introducing extrinsic linker-derived material into the reporter-targeting-moiety conjugate. The reactive moieties in a linking agent may be the same (homobifunctional agents) or different (heterobifunctional agents or, where several dissimilar reactive moieties are present, heteromultifunctional agents), providing a diversity of potential reagents that may bring about covalent bonding between any chemical species, either intramolecularly or intermolecularly.

In some embodiments it may be desirable to introduce labile linkages, e.g., containing spacer arms which are biodegradable or chemically sensitive or which incorporate enzymatic cleavage sites. Alternatively the spacer may include polymeric components, e.g., to act as surfactants and enhance the stability of the agent. The spacer may also contain reactive moieties, e.g., as described above to enhance surface crosslinking. Spacer elements may typically consist of aliphatic chains which effectively separate the reactive moieties of the linker. The spacer elements may also comprise macromolecular structures such as polyethylene glycols (PEGs) (see e.g., Milton Harris, J. (ed) “Poly (ethylene glycol) chemistry, biotechnical and biomedical applications” Plenum Press, New York, 1992). In particular embodiments, the molecular weights for PEG spacers used in accordance with the invention are between 120 Daltons and 20 kDaltons.

In some embodiments, the linkers useful in the practice of this invention derive from those groups which can react with any relevant molecule which comprises a targeting moiety containing a reactive group, whether or not such a molecule is a protein, to form a linking group. By way of example, where the reporter moiety is a chelated metal species (e.g., a paramagnetic metal ion or a metal radionuclide), the linker may comprise a chain attached to a metal chelating group, a polymeric chain with a plurality of metal chelating groups pendant from the molecular backbone or incorporated in the molecular backbone, a branched polymer with metal chelating groups at branch termini (e.g., a dendrimeric polychelant). In a particular embodiment, the linker simply binds the target and reporter moieties together such that the contrast agent can exert its desired effects, e.g., to enhance contrast in vivo during a diagnostic imaging procedure. Thus, in particular embodiments, the linker biodegrades after administration.

In some embodiments where the reporter moiety is a chelated metal ion, the linker group incorporates the chelant moiety. Alternatively, the chelated metal may be carried on or in a particulate reporter moiety. In either case, conventional metal chelating groups well known in the fields of radiopharmaceuticals and MRI contrast media are suitable for, e.g., linear, cyclic and branched polyamino-polycarboxylic acids and phosphorus oxyacid equivalents, and other sulphur and/or nitrogen ligands known in the art, eg. DTPA, DTPA-BMA, EDTA, DO3A, TMT (see e.g., U.S. Pat. No. 5,367,080), BAT and analogs (see e.g., Ohmono et al., J. Med. Chem. 35: 157-162 (1992) and Kung et al. J. Nucl. Med. 25: 326-332 (1984)), the N 2 S 2 chelant ECD of Neurolite, MAG (see e.g., Jurisson et al. Chem. Rev. 93: 1137-1156 (1993)), HIDA, DOXA (1-oxa-4,7,10-triazacyclododecanetriacetic acid), NOTA (1,4,7-triazacyclononanetriacetic acid), TETA (1,4,8,11-tetraazacyclotetradecanetetraacetic acid), or THT 4′-(3-amino-4-methoxy-phenyl)-6,6″-bis(N′,N′-dicarboxymethyl-N-methylhydrazino)-2,2′:6′,2″-terpyridine). Further suitable chelating agents are known in the art for diagnostic metals, eg. in MR, X-ray and radiodiagnostic agents (see e.g., U.S. Pat. No. 4,647,447, EP-A-71564, U.S. Pat. No. 4,687,659, WO89/00557, U.S. Pat. No. 4,885,363, and EP-A-232751).

The reporter moieties in the imaging agents of the present invention may be any moiety capable of detection either directly or indirectly in an in vivo diagnostic imaging procedure, e.g., moieties which emit or may be caused to emit detectable radiation (e.g., by radioactive decay, fluorescence excitation, or spin resonance excitation), moieties which affect local electromagnetic fields (e.g., paramagnetic, superparamagnetic, ferromagnetic, or ferromagnetic species), moieties which absorb or scatter radiation energy (e.g., chromophores, particles, including gas or liquid containing vesicles), heavy elements and compounds thereof, and moieties which generate a detectable substance (e.g., gas microbubble generators).

A wide range of materials detectable by diagnostic imaging modalities is known from the art and, therefore, a suitable reporter is selected according to the imaging modality to be used. For example, for ultrasound imaging an echogenic material, or a material capable of generating an echogenic signal is suitable; for X-ray imaging a suitable reporter is or contains a heavy atom (e.g., of atomic weight 38 or above); for MR imaging a suitable reporter is a non zero nuclear spin isotope (such as ¹⁹F) or a material having unpaired electron spins and is thus has paramagnetic, superparamagnetic, ferrimagnetic or ferromagnetic properties; for light imaging a suitable reporter is a light scatterer (e.g., a colored or uncolored particle), a light absorber or a light emitter; for magnetometric imaging a suitable reporter is one with detectable magnetic properties; for electrical impedance imaging a suitable reporter is one that affects electrical impedance; and for scintigraphy, SPECT, or PET, a suitable reporter is a radionuclide.

Examples of suitable reporters are known from the diagnostic imaging literature, e,g., magnetic iron oxide particles, gas-containing vesicles, chelated paramagnetic metals e.g., Gd, Dy, Mn, or Fe (see e.g., U.S. Pat. No. 4,647,447, WO97/25073, U.S. Pat. No. 4,863,715, U.S. Pat. No. 4,770,183, WO96/09840, WO85/02772, WO92/17212, WO97/29783, EP-A-554213, U.S. Pat. No. 5,228,446, WO91/15243, WO93/05818, WO96/23524, WO96/17628, U.S. Pat. No. 5,387,080, WO95/26205, and GB9624918.0).

In particular embodiments, the reporters are: chelated paramagnetic metal ions such as Gd, Dy, Fe, and Mn, particularly when chelated by macrocyclic chelant groups (e.g., tetraazacyclododecane chelants such as DOTA, D03A, HP-DO3A and analogs thereof), or when chelated by linker chelant groups such as DTPA, DTPA-BMA, EDTA, DPDP; metal radionuclide such as ⁹⁰Y, ^{99m}Tc, ¹¹¹In, ⁴⁷Sc, ⁶⁷/Ga, ⁵¹Cr, ^{177m}Sn, ⁶⁷Cu, ¹⁶⁷Tm, ⁹⁷Ru, ¹⁸⁸Re, ¹⁷⁷Lu, ¹⁹⁹Au, ²⁰³Pb and ¹⁴¹Ce; superparamagnetic iron oxide crystals; chromophores and fluorophores having absorption and/or emission maxima in the range 300-1400 nm, particularly 600 nm to 1200 nm, or 650 to 1000 nm; vesicles containing fluorinated gases, i.e., containing materials in the gas phase at 37° C., more particularly, fluorine containing, e.g., SF 6 or perfluorinated C 1-6 hydrocarbons or other gases and gas precursors described in WO97/29783); chelated heavy metal cluster ions (e.g., W or Mo polyoxoanions or the sulphur or mixed oxygen/sulphur analogs); or covalently bonded non-metal atoms which are either high atomic number (e.g., iodine), are radioactive, e.g., ¹²³I, ¹³¹I atoms; or iodinated compound containing vesicles.

The reporter may be: 1) a chelatable metal or polyatomic metal-containing ion (e.g., TcO), where the metal is a high atomic number metal (e.g., atomic number greater than 37), a paramagentic species (e.g., a transition metal or lanthanide), or a radioactive isotope; 2) a covalently bound non-metal species which is an unpaired electron site (e.g., an oxygen or carbon in a persistent free radical), a high atomic number non-metal, or a radioisotope; 3) a polyatomic cluster or crystal containing high atomic number atoms, displaying cooperative magnetic behaviour (e.g., superparamagnetism, ferrimagnetism or ferromagnetism) or containing radionuclides; 4) a gas or a gas precursor (i.e., a material or mixture of materials which is gaseous at 37° C.); 5) a chromophore (by which term species which are fluorescent or phosphorescent are included), eg. an inorganic or organic structure, particularly a complexed metal ion or an organic group having an extensive delocalized electron system; or 6) a structure or group having electrical impedance varying characteristics, e.g., by virtue of an extensive delocalized electron system.

Examples of suitable metal radionuclides include, but are not limited to, ⁹⁰Y, ^{99m}Tc, ¹¹¹In, ⁴⁷Sc, ⁶⁷Ga, ⁵¹Cr, ^{177m}Sn, ⁶⁷Cu, ¹⁶⁷Tm, ⁹⁷Ru, ¹⁸⁸Re, ¹⁷⁷Lu, ¹⁹⁹Au, ²⁰³Pb and ¹⁴¹Ce. Examples of suitable paramagnetic metal ions include, but are not limited to, ions of transition and lanthanide metals (e.g., metals having atomic numbers of 6 to 9, 21-29, 42, 43, 44, or 57-71); or ions of Cr, V, Mn, Fe, Co, Ni, Cu, La, Ce, Pr, Nd, Pm, Sm, Eu, Gd, Tb, Dy, Ho, Er, Tm, Yb and Lu. Examples of suitable metal radionuclides include, but are not limited to, fluorescent metal ions: lanthanides, La, Ce, Pr, Nd, Pm, Sm, Eu, Gd, Tb, Dy, Ho, Er, Tm, Yb, and Lu. Examples of suitable metal radionuclides include, but are not limited to, heavy metal-containing reporters such as atoms of Mo, Bi, Si, and W, and in particular may be polyatomic cluster ions (e.g., Bi compounds and W and Mo oxides) (see e.g., WO91/14460, WO92/17215, WO96/40287, and WO96/22914). Representative and suitable chelating groups are described in e.g., U.S. Pat. No. 5,559,214 A, WO 95/26754, WO 94/08624, WO 94/09056, WO 94/29333, WO 94/08624, WO 94/08629 A1, WO 94/13327 A1, and WO 94/12216 A1. Further examples of suitable chelant groups are described in U.S. Pat. No. 4,647,447, WO89/00557, U.S. Pat. No. 5,367,080, and U.S. Pat. No. 5,364,613.

In some embodiments, for diagnostic imaging, an imaging agent of the present invention comprising a targeting-linker-reporter moiety contains a ratio of metal radionuclide ion to chelating agent that is effective in such diagnostic imaging applications. In one embodiment, the mole ratio of metal ion per chelating agent is from about 1:1,000 to about 1:1. In some embodiments, in radiotherapeutic applications, an imaging agent of the present invention comprising a targeting-linker-reporter moiety, comprises a ratio of metal radionuclide ion to chelating agent that is effective in such therapeutic applications. In one embodiment, the mole ratio of metal ion per chelating agent is from about 1:100 to about 1:1. Examples of suitable radioisotopes include, but are not limited to, radioisotope of Sc, Fe, Pb, Ga, Y, Bi, Mn, Cu, Cr, Zn, Ge, Mo, Ru, Sn, Sr, Sm, Lu, Sb, W, Re, Po, Ta and Tl. In one embodiment, the radionuclide is ⁴⁴Sc, ⁶⁴Cu, ⁶⁷Cu, ²¹²Pb, ⁶⁸Ga, ⁹⁰Y, ¹⁵³Sm, ²¹²Bi, ¹⁸⁶Re, and ¹⁸⁸Re. These radioisotopes can be atomic or ionic.

In some embodiments, the isotopes or isotope pairs can be used for both imaging and therapy without having to change the radiolabeling methodology or chelator. Examples of such isotopes or isotope pairs include, but are not limited to, ⁴⁷Sc 21; ¹⁴¹Ce 58; ¹⁸⁸Re 75; ¹⁷⁷Lu 71; ¹⁹⁹Au 79; ⁴⁷Sc 21; ¹³¹I 53; ⁶⁷Cu 29; and ¹²³I 53; ¹⁸⁸Re 75, and ^{99m}Tc 43; ⁹⁰Y 39 and ⁸⁷Y 39; ⁴⁷Sc 21 and ⁴⁴Sc 21; ⁹⁰Y 39 and ¹²³I 53; ¹⁴⁶Sm 62 and Sm 62; and ⁹⁰Y 39 and ¹¹¹In 49.

Where the linker moiety contains a single chelant, that chelant may be attached directly to the targeting moiety, e.g., via one of the metal coordinating groups of the chelant which may form an ester, amide, thioester or thioamide bond with an amine, thiol or hydroxyl group on the vector. Alternatively the targeting moiety and chelant may be directly linked via a functionality attached to the chelant backbone, e.g., a CH 2-phenyl-NCS group attached to a ring carbon of DOTA (see e.g., Meares et al. (1988) JACS 110:6266-6267), or indirectly via a homo or hetero-bifunctional linker, e.g., a bis amine, bis epoxide, diol, diacid, or difunctionalised PEG. In this case, the bifunctional linker can provide a chain of 1 to 200, or more particularly 3 to 30 atoms, between a targeting moiety and the chelant residue.

Where the chelated species is carried by a particulate (or molecular aggregate, e.g., vesicular) linker, the chelate may for example be an unattached mono or polychelate (e.g., Gd DTPA-BMA or Gd HP-DO3A) enclosed within the particle, or it may be a mono or polychelate conjugated to the particle either by covalent bonding or by interaction of an anchor group (eg., a lipophilic group) on the mono/polychelate with the membrane of a vesicle (see e.g., WO96/11023).

Examples of suitable non-metal atomic reporters include, but are not limited to, radioisotopes such as ¹²³I and ¹³¹I as well as non zero nuclear spin atoms such as ¹⁸F, and heavy atoms such as I. In some embodiments, such reporters (including a plurality thereof, e.g. 2 to 200) may be covalently bonded to a linker backbone, either directly using conventional chemical synthesis techniques or via a supporting group (e.g. a triiodophenyl group). Such non-metal atomic reporters may be linked to the linker or carried in or on a particulate linker, e.g., in a vesicle (see e.g., WO95/26205 and GB9624918.0). Linkers of the type described in connection with the metal reporters may be used for non-metal atomic reporters with the non-metal atomic reporter or groups carrying such reporters taking the place of some or all of the chelant groups.

Preferred organic chromophoric and fluorophoric reporters include groups having a delocalized electron system, e.g., cyanines, merocyanines, phthalocyanines, naphthalocyanines, triphenylmethines, porphyrins, pyrilium dyes, thiapyrilium dyes, squarylium dyes, croconium dyes, azulenium dyes, indoanilines, benzophenoxazinium dyes, benzothiaphenothiazinium dyes, anthraquinones, napthoquinones, indathrenes, phthaloylacridones, trisphenoquinones, azo dyes, intramolecular and intermolecular charge-transfer dyes and dye complexes, tropones, tetrazines, bis(dithiolene) complexes, bis(benzene-dithiolate) complexes, iodoaniline dyes, and bis(S,O-dithiolene) complexes. Examples of suitable organic or metallated organic chromophores include, but are not limited to, those described in “Topics in Applied Chemistry: Infrared absorbing dyes” Ed. M. Matsuoka, Plenum, N.Y. 1990, “Topics in Applied Chemistry: The Chemistry and Application of Dyes”, Waring et al., Plenum, N.Y., 1990, “Handbook of Fluorescent Probes and Research Chemicals” Haugland, Molecular Probes Inc, 1996, DE-A-4445065, DE-A-4326466, JP-A-3/228046, Narayanan et al. J. Org. Chem. 60: 2391-2395 (1995), Lipowska et al. Heterocyclic Comm. 1: 427-430 (1995), Fabian et al. Chem. Rev. 92: 1197 (1992), WO96/23525, Strekowska et al. J. Org. Chem. 57: 4578-4580 (1992), and WO96/17628. Particular examples of suitable chromophores include, but are not limited to, xylene cyanole, fluorescein, dansyl, NBD, indocyanine green, DODCI, DTDCI, DOTCI, and DDTCI.

Representative examples of visible dyes include, but are not limited to, fluorescein derivatives, rhodamine derivatives, coumarins, azo dyes, metalizable dyes, anthraquinone dyes, benzodifuranone dyes, polycyclic aromatic carbonyl dyes, indigoid dyes, polymethine dyes, azacarbocyanine dyes, hemicyanine dyes, barbituates, diazahemicyanine dyes, stryrl dyes, diaryl carbonium dyes, triaryl carbonium dyes, phthalocyanine dyes, quinophthalone dyes, triphenodioxazine dyes, formazan dyes, phenothiazine dyes such as methylene blue, azure A, azure B, and azure C, oxazine dyes, thiazine dyes, naphtholactam dyes, diazahemicyanine dyes, azopyridone dyes, azobenzene dyes, mordant dyes, acid dyes, basic dyes, metallized and premetallized dyes, xanthene dyes, direct dyes, leuco dyes which can be oxidized to produce dyes with hues bathochromically shifted from those of the precursor leuco dyes, and other suitable dyes such as those described by Waring, D. R. and Hallas, G., in “The Chemistry and Application of Dyes”, Topics in Applied Chemistry, Plenum Press, New York, N.Y., 1990; and by Haugland, R. P., “Handbook of Fluorescent Probes and Research Chemicals”, Sixth Edition, Molecular Probes, Inc., Eugene Oreg., 1996.

Such chormophores and fluorophores may be covalently linked either directly to the targeting moiety or to or within a linker structure. Linkers of the type described herein connection with the metal reporters may be used for organic chromophores or fluorophores with the chromophores/fluorophores taking the place of some or all of the chelant groups. As with the metal chelants described herein chromophores or fluorophores may be carried in or on a particulate linker-reporter moieties, e.g., in or on a vesicle or covalently bonded to inert matrix particles that can also function as a light scattering reporter.

The particulate reporters and linker-reporter moieties generally fall into two categories—those where the particle comprises a matrix or shell which carries or contains the reporter and those where the particle matrix is itself the reporter. Examples of the first category include, but are not limited to: vesicles (e.g. micelles, liposomes, microballoons and microbubbles) containing a liquid, gas or solid phase which contains the contrast effective reporter, eg. an echogenic gas or a precursor therefor (see e.g., GB 9700699.3), a chelated paramagnetic metal or radionuclide, or a water-soluble iodinated X-ray contrast agent; porous particles loaded with the reporter, eg. paramagnetic metal loaded molecular sieve particles; and solid particles, e.g., of an inert biotolerable polymer, onto which the reporter is bound or coated, e.g., dye-loaded polymer particles. Examples of the second category include, but are not limited to: light scattering organic or inorganic particles; magnetic particles (ie. superparamagnetic, ferromagnetic or ferrimagnetic particles); and dye particles. In particular embodiments, the reporters or reporter-linkers comprise superparamagnetic particles (see e.g., U.S. Pat. No. 4,770,183, WO97/25073, WO96/09840), echogenic vesicles (see e.g., WO92/17212, WO97/29783), iodine-containing vesicles (see e.g., WO95/26205 and GB9624918.0), or dye-loaded polymer particles (see e.g., WO96/23524). The particulate reporters may have one or more targeting moieties attached directly or indirectly to their surfaces. In some embodiments a plurality (e.g., 2 to 50) of targeting moieties are attached per particle.

The imaging agents of the present invention may be administered to a subject for imaging in amounts sufficient to yield the desired contrast with the particular imaging modality. In particular embodiments where the reporter is a metal, dosages from about 0.001 to about 5.0 mmoles of chelated imaging metal ion per kilogram of patient bodyweight may be effective to achieve adequate contrast enhancements. In particular embodiments using MRI applications, dosages of imaging metal ion may be in the range from about 0.02 to about 1.2 mmoles/kg body weight, wherein for X-ray applications dosages from about 0.05 to 2.0 mmoles/kg are generally effective to achieve X-ray attenuation. In a particular embodiment, dosages for X-ray applications are from 0.1 to 1.2 mmoles of the lanthanide or heavy metal compound/kg bodyweight. In some embodiments where the reporter is a radionuclide, dosages of 0.01 to 100 mCi, and more particularly 0.1 to 50 mCi may be sufficient per 70 kg bodyweight. In another embodiment where the reporter is a superparamagnetic particle, the dosage may be e.g., 0.5 to 30 mg Fe/kg bodyweight. In another embodiment, where the reporter is a gas or gas generator, e.g., in a microballoon, the dosage may be e.g., 0.05 to 100 μL gas per 70 kig bodyweight.

In particular embodiments for the imaging of some portions of the body, a mode for administering contrast agents is parenteral, e.g., intravenous administration. Examples of parenterally administrable forms include, but are not limited to, intravenous solutions that are sterile and free from physiologically unacceptable agents, and have low osmolality to minimize irritation or other adverse effects upon administration, and thus the contrast medium is preferably isotonic or slightly hypertonic. Examples of suitable vehicles include, but are not limited to, aqueous vehicles customarily used for administering parenteral solutions such as Sodium Chloride Injection, Ringer's Injection, Dextrose Injection, Dextrose and Sodium Chloride Injection, Lactated Ringer's Injection and other solutions such as are described in Remington's Pharmaceutical Sciences, 15th ed., Easton: Mack Publishing Co., pp. 1405-1412 and 1461-1487 (1975) and The National Formulary XIV, 14th ed. Washington: American Pharmaceutical Association (1975). The solutions can further contain preservatives, antimicrobial agents, buffers and antioxidants conventionally used for parenteral solutions, excipients and other additives which are compatible with the chelates and which will not interfere with the manufacture, storage or use of products.

Thus, as described herein, the imaging agents of the present invention may be therapeutically effective in the treatment of inflammatory disease as well as detectable in in vivo imaging. Thus, for example the targeting moieties and/or the reporter moieties can have therapeutic efficacy, e.g., by virtue of the radiotherapeutic effect of a radionuclide reporter, the efficacy in photodynamic therapy of a chromophore (or fluorophore) reporter, or the therapeutic effect of a targeting moiety that modulates MDL-1 activity.

Assays and Kits

The present invention also relates to assays such as quantitative and qualitative assays for detecting levels of the protein, fragments or variants thereof in cells or in mammalian patient (e.g., a human, non-human primate, or rodent)s using the compositions of the present invention (e.g., an anti-MDL-1 polyclonal or monoclonal antibody) and kits comprising the compositions of the present invention, for carrying out such assays. More particularly, the present invention provides kits and methods for diagnosing and monitoring an inflammatory disease in a patient, including monitoring treatment of the disease, comprising the steps of: collecting a tissue sample from the patient; isolating monocytes from the sample; and measuring the levels of MDL-1 protein in the monocytes, wherein an increase in the levels of MDL-1 is indicative of an inflammatory response or disease. Further, macrophages and/or neutrophils can be isolated from the tissue sample and the levels of MDL-1 protein in the macrophages and/or neutrophils is measured. Any tissue sample may be used to measure macrophages e.g., bone marrow can be used. Alternatively, the levels of MDL-1 protein in the sample can be measured and a decrease in the levels of MDL-1 is indicative of an inflammatory response or disease.

Assay techniques that can be used to determine levels of a protein in a sample derived from a host are well-known to those of skill in the art. Such assay methods include radioimmunoassays, competitive-binding assays, Western Blot analysis and ELISA assays. In some embodiments, the assay comprises an ELISA assay and comprises preparing an antibody specific to MDL-1, preferably a monoclonal antibody. Additionally, in some embodiments, a reporter antibody that binds to the anti-MDL-1 monoclonal antibody, is prepared. The reporter antibody can then be attached to a detectable reagent such as a radioactive, fluorescent or enzymatic reagent, for example, horseradish peroxidase enzyme.

In some embodiments, in the ELISA assay, a sample is removed from a host and incubated on a solid support, e.g., a polystyrene dish, that binds the proteins in the sample. Free protein binding sites on the dish are then covered by incubating with a non-specific protein such as bovine serum albumin. The monoclonal antibody is then incubated in the dish and attach to MDL-1 protein attached to the polystyrene dish. Any unbound monoclonal antibody is then washed out with buffer, and thereafter the reporter antibody linked to horseradish peroxidase is placed in the dish resulting in binding of the reporter antibody to monoclonal antibody that is bound to the MDL-1. Unattached reporter antibody is then washed out, and reagents for peroxidase activity, including a calorimetric substrate, are then added to the dish, and the immobilized peroxidase, linked to the MDL-1 through the primary and secondary antibodies, produces a colored reaction product. The amount of color developed for a specified time period is indicative of the amount of the MDL-1 protein present in the sample, and quantitative results can be obtained by reference to a standard curve.

The present invention further provides methods for identification of binding molecules, e.g., ligands, to MDL-1. Genes encoding proteins for binding molecules to a cell surface receptor such as MDL-1, can be identified by a variety of methods known in the art e.g., ligand panning and FACS., and commonly described in laboratory manuals (see e.g., Coligan, et al., (1991) and Rivett, A. (1993)). For example, a yeast two-hybrid screen can be used to characterize the function of a protein by identifying other proteins with which it interacts. The protein of unknown function, can be produced as a chimeric protein additionally containing the DNA binding domain of GAL4. Plasmids containing nucleotide sequences which express this chimeric protein are transformed into yeast cells, which also contain a representative plasmid from a library containing the GAL4 activation domain fused to different nucleotide sequences encoding different potential target proteins. If the bait protein physically interacts with a target protein, the GAL4 activation domain and GAL4 DNA binding domain are tethered and are thereby able to act conjunctively to promote transcription of a reporter gene. If no interaction occurs between the chimeric protein and the potential target protein in a particular cell, the GAL4 components remain separate and unable to promote reporter gene transcription on their own.

Different reporter genes can be utilized, including β-galactosidase, HIS3, ADE2, or URA3. Also, multiple reporter sequences, each under the control of a different inducible promoter, can be utilized within the same cell to indicate interaction of the GAL4 components (and thus a specific target protein). Additionally, alternative DNA-binding domain or activation domain components may be used, such as LexA. For example, an activation domain may be paired with any DNA binding domain to generate transactivation of a reporter gene. Further, either of the two components may be of prokaryotic origin for transactivation of the reporter gene, as with the LexA system.

Reagents and design for two-hybrid systems are well known to those skilled in the art (see e.g., The Yeast Two-Hybrid System by P. L. Bartel and S. Fields (eds.) (Oxford University Press, 1997; Yang et al., 1995; James et al., 1996)), including improvements (Fashena et al., 2000). A variety to two-hybrid vectors are known, e.g., the Matchmaker® Systems from Clontech (Palo Alto, Calif.) or the HybriZAP® 2.1 Two Hybrid System (Stratagene; La Jolla, Calif.). Two-hybrid systems from other species are also known, e.g., mammals (Stratagene (La Jolla, Calif.)) or E. coli (Hu et al., 2000).

In an alternative embodiment, a two hybrid system is utilized wherein protein-protein interactions are detected in a cytoplasmic-based assay. In this embodiment, proteins are expressed in the cytoplasm, which allows posttranslational modifications to occur and permits transcriptional activators and inhibitors to be used as bait in the screen. An example of such a system is the CytoTrap® Two-Hybrid System (Stratagene). Suitable vectors (e.g., pMyr and pSos) and protocols are commercially available (Stratagene) and known (e.g., U.S. Pat. No. 5,776,689).

For example, screening for a protein that interacts with MDL-1 can comprise introducing into a cell a first nucleic acid comprising a DNA segment encoding a test protein, wherein the test protein is fused to a DNA binding domain, and a second nucleic acid comprising a DNA segment encoding at least part of MDL-1, respectively, wherein the at least part of MDL-1, respectively, is fused to a DNA activation domain. The interaction between the test peptide and the MDL-1 protein or fragment thereof can then be detected by assaying for an interaction between the DNA binding domain and the DNA activation domain.

Alternatively, λgt11, λ LZAP (Stratagene) or equivalent cDNA expression libraries can be screened using recombinant MDL-1. Recombinant MDL-1 protein or fragments thereof can be fused to small peptide tags such as FLAG, HSV or GST, and the peptide tags can comprise phosphorylation sites for a kinase such as heart muscle creatine kinase, or can be biotinylated. Also, recombinant MDL-1 can be e.g., phosphorylated with ³²P or used unlabeled and detected with streptavidin or antibodies against the tags. λgt11cDNA expression libraries can made from cells of interest and incubated with the recombinant MDL-1, washed and the cDNA clones that interact with MDL-1 isolated using known methods (e.g., see Sambrook (supra).

Another method is the screening of a mammalian expression library in which the cDNAs are cloned into a vector between a mammalian promoter and polyadenylation site and transiently transfected in cells. The binding protein can be detected by incubation of fixed and washed cells with a labeled MDL-1, and pools of cDNAs containing the cDNA encoding the binding protein of interest can be selected. The cDNA of interest can then be isolated by further subdivision of each pool followed by cycles of transient transfection, binding and autoradiography. Alternatively, the cDNA of interest can be isolated by transfecting the entire cDNA library into mammalian cells and panning the cells on a dish containing the MDL-1 bound to the plate. Cells which attach after washing are lysed and the plasmid DNA isolated, amplified in bacteria, and the cycle of transfection and panning repeated until a single cDNA clone is obtained. See Seed et al., 1987 and Aruffo et al., 1987 which are herein incorporated by reference. If the binding protein is secreted, its cDNA can be obtained by a similar pooling strategy once a binding or neutralizing assay has been established for assaying supernatants from transiently transfected cells. General methods for screening supernatants are known (see e.g., Wong et al., (1985)).

Another alternative method is the isolation of proteins interacting with MDL-1 directly from cells. Fusion proteins of MDL-1 with GST or small peptide tags can be made and immobilized on beads. Biosynthetically labeled or unlabeled protein extracts from the cells of interest can then be prepared, incubated with the beads and washed with buffer. Proteins interacting with the MDL-1 can then be eluted specifically from the beads and analyzed by SDS-PAGE (sodium dodecyl sulphate polyacrylamide gel electrophoresis). The primary amino acid sequence of a binding partner can be obtained by sequencing. Optionally, the cells can be treated with agents that induce a functional response such as tyrosine phosphorylation of cellular proteins. An example of such an agent would be a growth factor or cytokine.

Another alternative method is immunoaffinity purification. Recombinant MDL-1 can be incubated with labeled or unlabeled cell extracts and immunoprecipitated with anti-MDL-1 antibodies. The immunoprecipitate can then be recovered with protein A Sepharose and analyzed by SDS-PAGE. The unlabelled proteins can be labeled by biotinylation and detected on SDS gels e.g., with streptavidin. The binding partner proteins can then be analyzed by sequencing. Standard and known biochemical purification steps can be employed prior to sequencing.

Another suitable method for identifying and isolating an MDL-1 ligand, is the screening of peptide libraries for binding partners. For example, recombinant tagged or labeled MDL-1 can be used to select peptides from a peptide or phosphopeptide library which interact with the MDL-1. Sequencing of the peptides can provide identification of consensus peptide sequences found in proteins that interact with MDL-1.

Synthesis and Production

Methods of synthesizing and producing amino acid, nucleic acid, and chemical compositions are well known in the art and can be used to synthesize and produce the compositions of the present invention.

In particular, a protein composition of the present invention can be constructed and produced using known methods e.g., by recombinant or synthetic nucleic acid or protein methods. Techniques for nucleic acid manipulation and expression are described generally, for example, in Sambrook, et al. (1989) Molecular Cloning: A Laboratory Manual (2d ed.) Vols. 1-3, Cold Spring Harbor Laboratory. Techniques for synthesis of proteins are described, for example, in Merrifield (1963) J. Amer. Chem. Soc. 85:2149-2156; Merrifield (1986) Science 232:341-347; and Atherton, et al. (1989) Solid Phase Peptide Synthesis: A Practical Approach, IRL Press, Oxford.

For example, the protein compositions of the present invention can be produced in suitable cells or cell lines such as human, rodent, or other mammalian cell lines. Suitable cell lines include, but are not limited to, baby hamster kidney (BHK) cells, Chinese hamster ovary (CHO) cells (including dihydrofolate reductase (DHFR)-deficient cells), and COS cells.

Suitable vectors include, but are not limited to, those suitable for expression in a mammalian host cell. The vectors can be, for example, plasmids or viruses. The vector will typically contain the following elements: promoter and other “upstream” regulatory elements, origin of replication, ribosome binding site, transcription termination site, polylinker site, and selectable marker that are compatible with use in a mammalian host cell. Vectors may also contain elements that allow propagation and maintenance in prokaryotic host cells as well.

Examples of suitable vectors for use in the production of a composition of the present invention include, but are not limited to the vector of SEQ ID NO: 19 (pPEP-hFc-MDL-1) encoding a human Fc-MDL-1 fusion protein; and SEQ ID NO: 20 (pPEP1-mFc-MDL-1) encoding a mouse MDL-1 fusion protein. Thus, the present invention also provides vectors for expression of compositions that modulate MDL-1 activity in a cell, in vivo or in vitro.

Vectors encoding a protein of the present invention can be introduced into host cells by standard cell biology techniques, including transfection and viral techniques. By transfection is meant the transfer of genetic information to a cell using isolated DNA, RNA, or synthetic nucleotide polymer. Suitable transfection methods include, but are not limited to, calcium phosphate-mediated co-precipitation (Sambrook et al. (1989) Molecular Cloning: A Laboratory Manual, 2.sup.nd ed., Cold Spring Harbor Laboratory Press), lipofection (e.g., Lipofectamine Plus from Life Technologies of Rockville, Md.), DEAE-dextran-mediated transfection techniques, lysozyme fusion or erythrocyte fusion, scraping, direct uptake, osmotic or sucrose shock, direct microinjection, indirect microinjection such as via erythrocyte-mediated techniques, protoplast fusion, or by subjecting the host cells to electric currents (e.g., electroporation), to name but a few. The above list of transfection methods are exemplary and not exhaustive.

To facilitate selection of the host cells containing the nucleic acid encoding a protein of the present invention, the nucleic acid encoding the protein can be introduced with a selection marker. The selection marker can be encoded by a nucleic acid sequence present on the same expression vector encoding the protein. Alternatively, the selection marker can be encoded by a nucleic acid sequence present on a different vector. In the latter case, the two vectors can be co-introduced into the host cells by either cotransfection or co-transduction. Suitable selection markers include, for example, Hygromycin B (Hyg B) and dihydrofolate reductase (DHFR).

Transiently or stably transfected cells can be used for the production and analysis of a protein of the present invention. The stably maintained nucleic acid can be present in any of various configurations in the host cell. For example, in one embodiment, the stably maintained nucleic acid sequence is integrated in a chromosome of a host cell. In other embodiments, the stably maintained nucleic acid sequence can be present as an extrachromosomal array, as an artificial chromosome, or in another suitable configuration. The host cells containing the nucleic acid sequence encoding the protein can be maintained under conditions suitable for expression of the protein, and standard cell culture methods, conditions and media can be used for maintaining the host cells expressing the protein.

In one embodiment, CHO cells are used to produce a protein of the present invention. In order to obtain a stably transfected CHO cell, a nucleic acid sequence encoding the protein and a nucleic acid sequence encoding a selection marker are introduced into CHO cells, e.g., by electroporation, protoplast fusion or lipofection methods. The nucleic acid sequence encoding the protein and the nucleic acid sequence encoding a selection marker can be present on the same expression vector. Alternatively, the nucleic acid sequence encoding the protein and the nucleic acid sequence encoding a selection marker can be present on separate vectors. Examples of selection markers, include but are not limited to, Hyb B, and DHFR. Stably transfected clones can be isolated and propagated in the presence of Hyg B at a suitable concentration (e.g., 200, 250, or 300 micrograms/ml), in standard tissue culture medium (e.g., MEM+FBS, DMEM/F-12 medium, or VP-SFM available from Life Technologies), and other suitable media. The expression levels of the protein can be monitored by standard protein-detection assays (e.g., by ELISA, Western Blot, dot blot, or other known or standard protein detection assays) using samples from supernatants and culture media, and the clones selected for propagation.

Purification of a composition of the present invention can be performed using standard and known methods and preferably, using GMP procedures. The protein can be purified to homogeneity or near homogeneity using known methods, e.g., chromatographic purification. For example, a purification scheme for a protein may include, but is not limited to, a protein capture, concentration, and/or formulation step. For example, chromatography resin materials that bind to the Fc portion of an Fc-MDL-1 fusion protein of the present invention can be used to capture such proteins. Suitable resin materials include, but are not limited to, resins coupled to Protein A. Further steps may be included to remove contaminating components. For example, hydroxyapatite chromatography, Sepharose Q chromatography, size exclusion chromatography, or hydrophobic interaction chromatography may be used to remove contaminants and, further, the purified protein can be concentrated to a desired concentration using ultrafiltration; diafiltered into a suitable formulation buffer; filter sterilized; and dispensed into vials.

The compositions of the present invention also include polyclonal or monoclonal antibodies (e.g., anti-MDL-1 antibodies). The antibodies of the present invention encompass fragments, fusions, and variants of antibodies, and humanized forms thereof (e.g., fusion proteins comprising an antibody portion and an MDL-1 portion). These antibodies are suitable for use in various diagnostic or therapeutic applications as described herein, including in the Examples. The techniques for preparing and using various antibody-based constructs and fragments are well known in the art. Means for preparing and characterizing antibodies are also well known in the art (See, e.g., Antibodies: A Laboratory Manual, Cold Spring Harbor Laboratory, 1988; incorporated herein by reference).

Antibodies can be raised to MDL-1, including the various allelic or species variants of MDL-1 and fragments thereof, both in their naturally occurring forms and in their recombinant forms. Additionally, antibodies can be raised to MDL-1 in either the active forms or inactive forms, or native or denatured forms. Anti-idiotypic antibodies are also contemplated.

Antibodies, including binding fragments and single chain versions, against predetermined fragments of MDL-1 can be raised by immunization of animals with conjugates of the fragments with immunogenic proteins. Monoclonal antibodies are prepared from cells secreting the desired antibody. These antibodies can be screened for binding to normal or defective MDL, or screened for agonistic or antagonistic functional activity.

The antibodies, including antigen binding fragments, of the present invention can have significant diagnostic or therapeutic value. They can be potent antagonists that bind to MDL-1 and/or inhibit partner or ligand binding or inhibit the ability to signal transduce. The antibodies can also be useful as non-neutralizing antibodies and can be coupled to toxins or radionuclides so that when the antibody binds to the antigen, the cell itself is killed. Further, these antibodies can be conjugated to drugs or other therapeutic agents, either directly or indirectly e.g., by means of a linker.

The antibodies of the present invention can also be useful in diagnostic applications e.g., as capture or non-neutralizing antibodies that bind MDL-1 without inhibiting partner or ligand binding and/or signal transduction. Such neutralizing antibodies can be useful in competitive binding assays and in detecting or quantifying MDL-1 and/or its partners or ligands.

Further MDL-1 fragments can be joined to other materials, particularly proteins, as fused or covalently joined proteins to be used as immunogens. An MDL-1 and fragments thereof can be fused or covalently linked to a variety of immunogens, such as keyhole limpet hemocyanin, bovine serum albumin, tetanus toxoid, etc. See Microbiology, Hoeber Medical Division, Harper and Row, 1969; Landsteiner (1962) Specificity of Serological Reactions, Dover Publications, New York, and Williams, et al. (1967) Methods in Immunology and Immunochemistry, Vol. 1, Academic Press, New York, for descriptions of methods of preparing polyclonal antisera. Examples of such methods include, but are not limited to, hyperimmunization of an animal with an antigen, where the blood of the animal is collected shortly after the repeated immunizations and the gamma globulin isolated. Alternatively, cells may be collected for producing hybridomas.

In particular embodiments, it is desirable to prepare monoclonal antibodies from various mammalian hosts, such as mice, rodents, primates, humans, etc. Description of techniques for preparing such monoclonal antibodies may be found in, e.g., Stites, et al. (eds.) Basic and Clinical Immunology (4th ed.), Lange Medical Publications, Los Altos, Calif., and references cited therein; Harlow and Lane (1988) Antibodies: A Laboratory Manual, CSH Press; Goding (1986) Monoclonal Antibodies: Principles and Practice (2d ed.) Academic Press, New York; and particularly in Kohler and Milstein (1975) in Nature 256:495-497. Examples of such methods include, but are not limited to, injecting an animal with an immunogen. The animal is then sacrificed and cells taken from its spleen, which are then fused with myeloma cells to produce a hybrid cell or “hybridoma” that is capable of reproducing in vitro. The population of hybridomas is then screened to isolate individual clones, each of which secrete a single antibody species to the immunogen. In this manner, the individual antibody species obtained are the products of immortalized and cloned single B cells from the immune animal generated in response to a specific site recognized on the immunogenic substance.

Other suitable techniques involve in vitro exposure of lymphocytes to the antigenic proteins or alternatively to selection of libraries of antibodies in phage or similar vectors (see, e.g., Huse, et al. (1989) “Generation of a Large Combinatorial Library of the Immunoglobulin Repertoire in Phage Lambda,” Science 246:1275-1281; and Ward, et al. (1989) Nature 341:544-546). The proteins and antibodies of the present invention may be used with or without modification, including chimeric or humanized antibodies. Also, the proteins and antibodies can be labeled by joining, either covalently or non-covalently, a substance which provides for a detectable signal. A wide variety of labels and conjugation techniques are known and are reported extensively in both the scientific and patent literature. Suitable labels include radionuclides, enzymes, substrates, cofactors, inhibitors, fluorescent moieties, chemiluminescent moieties, magnetic particles, and the like. Patents, teaching the use of such labels include U.S. Pat. Nos. 3,817,837; 3,850,752; 3,939,350; 3,996,345; 4,277,437; 4,275,149; and 4,366,241. Also, recombinant immunoglobulins may be produced, see Cabilly, U.S. Pat. No. 4,816,567.

As described herein, the antibodies of the present invention can also be used for affinity chromatography in isolating the protein. Columns can be prepared where the antibodies are linked to a solid support, e.g., particles, such as agarose, SEPHADEX, or the like, where a cell lysate may be passed through the column, the column washed, followed by increasing concentrations of a mild denaturant, whereby the purified MDL-1 protein can be released.

Also as described herein, the antibodies may also be used to screen expression libraries for particular expression products. Usually the antibodies used in such a procedure can be labeled with a moiety allowing easy detection of presence of antigen by antibody binding.

Antibodies raised against an MDL-1 can also be used to raise anti-idiotypic antibodies. These can be useful in detecting or diagnosing various immunological conditions related to inflammatory disease.

Compositions of the present invention for modulating MDL-1 activity also encompass nucleic acid molecules e.g., short interfering nucleic acid (siNA), short interfering RNA (siRNA), double-stranded RNA (dsRNA), micro-RNA (miRNa) and short hairpin RNA (shRNA) molecules capable of mediating RNA interference (RNAi) against MDL-1 and/or MDL-1 gene expression, in a cell, in vivo or in vitro, thereby, modulating MDL-1 activity. Such nucleic acid compositions can be constructed and produced using known methods in the art (see, e.g., U.S. Application Serial Nos. 10/826,966 and 10/757,803).

RNA interference refers to the process of sequence-specific post-transcriptional gene silencing in animals mediated by short interfering RNAs (siRNAs) (Zamore et al., 2000, Cell, 101, 25-33; Fire et al., 1998, Nature, 391, 806; Hamilton et al., 1999, Science, 286, 950-951; Lin et al., 1999, Nature, 402, 128-129; and Strauss, 1999, Science, 286, 886). The presence of long dsRNAs in cells stimulates the activity of a ribonuclease III enzyme referred to as dicer (Bass, 2000, Cell, 101, 235; Zamore et al., 2000, Cell, 101, 25-33; Hammond et al., 2000, Nature, 404, 293). Dicer is involved in the processing of the dsRNA into short pieces of dsRNA known as short interfering RNAs (siRNAs) (Zamore et al., 2000, Cell, 101, 25-33; Bass, 2000, Cell, 101, 235; Berstein et al., 2001, Nature, 409, 363). Short interfering RNAs derived from dicer activity are typically about 21 to about 23 nucleotides in length and comprise about 19 base pair duplexes (Zamore et al., 2000, Cell, 101, 25-33; Elbashir et al., 2001, Genes Dev., 15, 188). Dicer has also been implicated in the excision of 21- and 22-nucleotide small temporal RNAs (stRNAs) from precursor RNA of conserved structure that are implicated in translational control (Hutvagner et al., 2001, Science, 293, 834). The RNAi response also features an endonuclease complex, commonly referred to as an RNA-induced silencing complex (RISC), which mediates cleavage of single-stranded RNA having sequence complementary to the antisense strand of the siRNA duplex. Cleavage of the target RNA typically takes place in the middle of the region complementary to the antisense strand of the siRNA duplex (Elbashir et al., 2001, Genes Dev., 15, 188).

RNAi has been studied in a variety of systems (see, e.g., Fire et al. (1998) Nature, 391, 806 re RNAi in C. elegans. Bahramian; and Zarbl (1999) Molecular and Cellular Biology, 19, 274-283 and Wianny and Goetz (1999) Nature Cell Biol., 2, 70, reRNAi mediated by dsRNA in mammalian systems; Hammond et al. (2000) Nature, 404, 293 re RNAi in Drosophila cells transfected with dsRNA; Elbashir et al. (2001) Nature, 411, 494 and Tuschl et al., International PCT Publication No. WO 01/75164 re RNAi induced by introduction of duplexes of synthetic 21-nucleotide RNAs in cultured mammalian cells including human embryonic kidney and HeLa cells). Recent work in Drosophila embryonic lysates (see e.g., Elbashir et al. (2001) EMBO J., 20, 6877 and Tuschl et al., International PCT Publication No. WO 01/75164) has revealed particular siRNA length, structure, chemical composition, and sequence that are important for mediating efficient RNAi activity. These studies report that 21-nucleotide siRNA duplexes are most active when containing 3′-terminal dinucleotide overhangs. Furthermore, complete substitution of one or both siRNA strands with 2′-deoxy (2′-H) or 2′-O— methyl nucleotides abolishes RNAi activity, whereas substitution of the 3′-terminal siRNA overhang nucleotides with 2′-deoxy nucleotides (2′-H) was shown to be tolerated. Single mismatch sequences in the center of the siRNA duplex were also shown to abolish RNAi activity. In addition, these studies also indicate that the position of the cleavage site in the target RNA is defined by the 5′-end of the siRNA guide sequence rather than the 3′-end of the guide sequence (Elbashir et al., 2001, EMBO J., 20, 6877). Other studies have indicated that a 5′-phosphate on the target-complementary strand of a siRNA duplex is required for siRNA activity and that ATP is utilized to maintain the 5′-phosphate moiety on the siRNA (Nykanen et al., 2001, Cell, 107, 309).

Studies have also reported that replacing the 3′-terminal nucleotide overhanging segments of a 21-mer siRNA duplex having two-nucleotide 3′-overhangs with deoxyribonucleotides does not have an adverse effect on RNAi activity. Replacing up to four nucleotides on each end of the siRNA with deoxyribonucleotides has been reported to be well tolerated, whereas complete substitution with deoxyribonucleotides results in no RNAi activity (Elbashir et al., 2001, EMBO J, 20, 6877 and Tuschl et al., International PCT Publication No. WO 01/75164). In addition, Elbashir et al., supra, also report that substitution of siRNA with 2′-O-methyl nucleotides completely abolishes RNAi activity. Li et al., International PCT Publication No. WO 00/44914, and Beach et al., International PCT Publication No. WO 01/68836 preliminarily suggest that siRNA may include modifications to either the phosphate-sugar backbone or the nucleoside to include at least one of a nitrogen or sulfur heteroatom, however, neither application postulates to what extent such modifications would be tolerated in siRNA molecules, nor provides any further guidance or examples of such modified siRNA. Kreutzer et al., Canadian Patent Application No. 2,359,180, also describe certain chemical modifications for use in dsRNA constructs in order to counteract activation of double-stranded RNA-dependent protein kinase PKR, specifically 2′-amino or 2′-O-methyl nucleotides, and nucleotides containing a 2′-O or 4′-C methylene bridge. However, Kreutzer et al. similarly fails to provide examples or guidance as to what extent these modifications would be tolerated in dsRNA molecules.

Parrish et al., 2000, Molecular Cell, 6, 1077-1087, tested certain chemical modifications targeting the unc-22 gene in C. elegans using long (>25 nt) siRNA transcripts. The authors describe the introduction of thiophosphate residues into these siRNA transcripts by incorporating thiophosphate nucleotide analogs with T7 and T3 RNA polymerase and observed that RNAs with two phosphorothioate modified bases also had substantial decreases in effectiveness as RNAi. Further, Parrish et al. reported that phosphorothioate modification of more than two residues greatly destabilized the RNAs in vitro such that interference activities could not be assayed. Id. at 1081. The authors also tested certain modifications at the 2′-position of the nucleotide sugar in the long siRNA transcripts and found that substituting deoxynucleotides for ribonucleotides produced a substantial decrease in interference activity, especially in the case of Uridine to Thymidine and/or Cytidine to deoxy-Cytidine substitutions. Id. In addition, the authors tested certain base modifications, including substituting, in sense and antisense strands of the siRNA, 4-thiouracil, 5-bromouracil, 5-iodouracil, and 3-(aminoallyl) uracil for uracil, and inosine for guanosine. Whereas 4-thiouracil and 5-bromouracil substitution appeared to be tolerated, Parrish reported that inosine produced a substantial decrease in interference activity when incorporated in either strand. Parrish also reported that incorporation of 5-iodouracil and 3-(aminoallyl)uracil in the antisense strand resulted in a substantial decrease in RNAi activity as well.

The use of longer dsRNA has been described. For example, Beach et al., International PCT Publication No. WO 01/68836, describes specific methods for attenuating gene expression using endogenously-derived dsRNA. Tuschl et al., International PCT Publication No. WO 01/75164, describe a Drosophila in vitro RNAi system and the use of specific siRNA molecules for certain functional genomic and certain therapeutic applications; although Tuschl, 2001, Chem. Biochem., 2, 239-245, doubts that RNAi can be used to cure genetic diseases or viral infection due to the danger of activating interferon response. Li et al., International PCT Publication No. WO 00/44914, describe the use of specific long (141 bp-488 bp) enzymatically synthesized or vector expressed dsRNAs for attenuating the expression of certain target genes. Zernicka-Goetz et al., International PCT Publication No. WO 01/36646, describe certain methods for inhibiting the expression of particular genes in mammalian cells using certain long (550 bp-714 bp), enzymatically synthesized or vector expressed dsRNA molecules. Fire et al., International PCT Publication No. WO 99/32619, describe particular methods for introducing certain long dsRNA molecules into cells for use in inhibiting gene expression in nematodes. Plaetinck et al., International PCT Publication No. WO 00/01846, describe certain methods for identifying specific genes responsible for conferring a particular phenotype in a cell using specific long dsRNA molecules. Mello et al., International PCT Publication No. WO 01/29058, describe the identification of specific genes involved in dsRNA-mediated RNAi. Pachuck et al., International PCT Publication No. WO 00/63364, describe certain long (at least 200 nucleotide) dsRNA constructs. Deschamps Depaillette et al., International PCT Publication No. WO 99/07409, describe specific compositions consisting of particular dsRNA molecules combined with certain anti-viral agents. Waterhouse et al., International PCT Publication No. 99/53050 and 1998, PNAS, 95, 13959-13964, describe certain methods for decreasing the phenotypic expression of a nucleic acid in plant cells using certain dsRNAs. Driscoll et al., International PCT Publication No. WO 01/49844, describe specific DNA expression constructs for use in facilitating gene silencing in targeted organisms.

Others have reported on various RNAi and gene-silencing systems. For example, Parrish et al., 2000, Molecular Cell, 6, 1077-1087, describe specific chemically-modified dsRNA constructs targeting the unc-22 gene of C. elegans. Grossniklaus, International PCT Publication No. WO 01/38551, describes certain methods for regulating polycomb gene expression in plants using certain dsRNAs. Churikov et al., International PCT Publication No. WO 01/42443, describe certain methods for modifying genetic characteristics of an organism using certain dsRNAs. Cogoni et al., International PCT Publication No. WO 01/53475, describe certain methods for isolating a Neurospora silencing gene and uses thereof. Reed et al., International PCT Publication No. WO 01/68836, describe certain methods for gene silencing in plants. Honer et al., International PCT Publication No. WO 01/70944, describe certain methods of drug screening using transgenic nematodes as Parkinson's Disease models using certain dsRNAs. Deak et al., International PCT Publication No. WO 01/72774, describe certain Drosophila-derived gene products that may be related to RNAi in Drosophila. Arndt et al., International PCT Publication No. WO 01/92513 describe certain methods for mediating gene suppression by using factors that enhance RNAi. Tuschl et al., International PCT Publication No. WO 02/44321, describe certain synthetic siRNA constructs. Pachuk et al., International PCT Publication No. WO 00/63364, and Satishchandran et al., International PCT Publication No. WO 01/04313, describe certain methods and compositions for inhibiting the function of certain polynucleotide sequences using certain long (over 250 bp), vector expressed dsRNAs. Echeverri et al., International PCT Publication No. WO 02/38805, describe certain C. elegans genes identified via RNAi. Kreutzer et al., International PCT Publications Nos. WO 02/055692, WO 02/055693, and EP 1144623 B1 describes certain methods for inhibiting gene expression using dsRNA. Graham et al., International PCT Publications Nos. WO 99/49029 and WO 01/70949, and AU 4037501 describe certain vector expressed siRNA molecules. Fire et al., U.S. Pat. No. 6,506,559, describe certain methods for inhibiting gene expression in vitro using certain long dsRNA (299 bp-103.3 bp) constructs that mediate RNAi. Martinez et al., 2002, Cell, 110, 563-574, describe certain single stranded siRNA constructs, including certain 5′-phosphorylated single stranded siRNAs that mediate RNA interference in Hela cells. Harborth et al., 2003, Antisense & Nucleic Acid Drug Development, 13, 83-105, describe certain chemically and structurally modified siRNA molecules. Chiu and Rana, 2003, RNA, 9, 1034-1048, describe certain chemically and structurally modified siRNA molecules. Woolf et al., International PCT Publication Nos. WO 03/064626 and WO 03/064625 describe certain chemically modified dsRNA constructs.

Formulations

Examples of suitable formulations of a composition of the present invention include, but are not limited to, a buffer and a surfactant in liquid or in solid form. Examples of solid formulations include, but are not limited to, freeze-dried, spray-freeze-dried or spray-dried formulations. Examples of liquid formulations include, but are not limited to, those based on water, but can contain other components, such as, for example, ethanol, propanol, propanediol or glycerol, to name but a few.

The compositions of the present invention are preferably formulated in aqueous solutions following standard GMP procedures known to persons skilled in the art. Generally, a formulation is generated by mixing defined volumes of aqueous solutions comprising suitable constituents at suitable concentrations. For example, in some embodiments, a formulation contains a protein of the present invention (e.g., Fc-MDl-1 fusion protein or anti-MDL-1 antibody) at a concentration from 0.1 to 200 mg/ml (weight/volume). In various embodiments, a formulation contains a protein of the present invention at a concentration from about 0.1 mg/ml to about 0.25 mg/ml, about 0.25 mg/ml to about 0.5 mg/ml, about 0.5 mg/ml to about 1 mg/ml, about 1 mg/ml to about 5 mg/ml, about 5 mg/ml to about 10 mg/ml, about 10 mg/ml to about 20 mg/ml, about 20 mg/ml to about 30 mg/ml, about 30 mg/ml to about 40 mg/ml, about 40 mg/ml to about 50 mg/ml, about 50 mg/ml to about 100 mg/ml, about 100 mg/ml to about 150 mg/ml, and about 150 mg/ml to about 200 mg/ml.

Buffer components include any physiologically compatible substances that are capable of regulating pH, such as, for example, citrate salts, acetate salts, histidine salts, succinate salts, maleate salts, phosphate salts, lactate salts, their respective acids or bases or mixtures thereof. Commonly used buffer components are citrate salts and/or their free acid. A formulation typically contains a buffer component at a concentration from 10 to 100 mmol/l, preferably from 2 to 20 mmol/l, more preferably 10 mmol/l. In some embodiments, the pH of a formulation of the present invention is in the range from about 3.0 to 9.0. Examples of suitable pH ranges include, but are not limited to, about 3.0 to about 4.0, about 4.0 to about 5.0, about 5.0 to about 6.0, about 6.0 to about 7.0, about 7.0 to about 8.0, and about 8.0 to about 9.0.

Surfactants for Fc-MDL-1 formulations can be any excipient used as surfactants in pharmaceutical compositions, preferably polyethylene-sorbitane-esters (Tweens®), such as, Polyoxyethylene(20)-sorbitanmonolaurate, Polyoxyethylene(20)-sorbitanemon-opalmitate and Polyoxyethylene(20)-sorbitanemonostearate, and polyoxytheylene-polyoxypropylene-copolymers. A formulation typically contains a surfactant at a concentration from 0.001 to 1.0% w/v, preferably from 0.005 to 0.1% w/v, more preferably from 0.01 to 0.5% w/v.

A formulation can also contain one or more amino acids. Suitable amino acids include, but are not limited to, arginine, histidine, ornithine, lysine, glycine, methionine, isoleucine, leucine, alanine, phenylalanine, tyrosine, and tryptophan. In one embodiment, a formulation of a composition of the present invention contains glycine. Preferably, amino acids are used in salt forms, for example, a hydrochloride salt. Applicable amino acid concentrations range from 2 to 200 mmol/L, or from 50 to 150 mmol/L.

Additionally, a formulation can contain sugars such as sucrose, trehalose, sorbitol; antioxidants such as ascorbic acid or glutathion; preservatives such as phenol, m-cresol, methyl- or propylparabene; chlorbutanol; thiomersal; benzalkoniumchloride; polyethyleneglycols; cyclodextrins and other suitable components.

It is desirable that a formulation of the present invention is isotonic. For example, osmolality of a formulation can range from 150 to 450 mOsmol/kg. Pharmaceutical formulations have to be stable for the desired shelf-life at the desired storage temperature, such as at 2-8.degree. C., or at room temperature. A useful formulation of the present invention is well tolerated physiologically, easy to produce, can be dosed accurately, and is stable during storage at 2° C., 8° C., or 25° C., during multiple freeze-thaw cycles and mechanical stress, as well as other stresses such as storage for at least 3 months at 40° C.

Administration

The therapeutic compositions containing compositions of the present invention can be administered to a mammalian host or patient by any route. Thus, as appropriate, administration can be oral or parenteral (e.g., i.v., i.a., s.c., i.m.), including intravenous and intraperitoneal routes of administration. In addition, administration can be by periodic injections of a bolus of the therapeutics or can be made more continuous by intravenous or intraperitoneal administration from a reservoir which is external (e.g., an i.v. bag). In particular embodiments, the therapeutics of the instant invention can be pharmaceutical-grade. That is, particular embodiments comply with standards of purity and quality control required for administration to humans.

The formulations, for human medical use, of the therapeutics according to the present invention typically include such therapeutics in association with a pharmaceutically-acceptable carrier and optionally other ingredient(s). The carrier(s) can be “acceptable” in the sense of being compatible with the other ingredients of the formulations and not deleterious to the recipient thereof. Pharmaceutically acceptable carriers, in this regard, are intended to include any and all solvents, dispersion media, coatings, antibacterial and antifungal agents, isotonic and absorption delaying agents, and the like, compatible with pharmaceutical administration. The use of such media and agents for pharmaceutically active substances is known in the art. Except insofar as any conventional media or agent is incompatible with the active compound, use thereof in the compositions is contemplated. Supplementary active compounds also can be incorporated into the compositions. The formulations can conveniently be presented in dosage unit form and can be prepared by any of the methods well known in the art of pharmacy/microbiology. In general, some formulations are prepared by bringing the therapeutics into association with a liquid carrier or a finely divided solid carrier or both, and then, if necessary, shaping the product into the desired formulation.

A pharmaceutical composition of the invention is formulated to be compatible with its intended route of administration. Examples of routes of administration include oral or parenteral, e.g., intravenous, intradermal, inhalation (e.g., after nebulization), transdermal (topical), transmucosal, nasal, buccal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide.

A preferred method for administration of compositions of the present invention is by parenteral (e.g., IV, IM, SC, or IP) routes and the compositions administered would ordinarily include therapeutically effective amounts of product in combination with acceptable diluents, carriers and/or adjuvants. Effective dosages are expected to vary substantially depending upon the condition treated but therapeutic doses are presently expected to be in the range of 0.2 to 2 mcg/kg body weight of the active material. Standard diluents such as human serum albumin are contemplated for pharmaceutical compositions of the invention, as are standard carriers such as saline.

Useful solutions for oral or parenteral administration can be prepared by any of the methods well known in the pharmaceutical art, described, for example, in Remington's Pharmaceutical Sciences, (Gennaro, A., ed.), Mack Pub., 1990. Formulations for parenteral administration also can include glycocholate for buccal administration, methoxysalicylate for rectal administration, or citric acid for vaginal administration. The parenteral preparation can be enclosed in ampoules, disposable syringes or multiple dose vials made of glass or plastic. Suppositories for rectal administration also can be prepared by mixing the drug with a non-irritating excipient such as cocoa butter, other glycerides, or other compositions that are solid at room temperature and liquid at body temperatures. Formulations also can include, for example, polyalkylene glycols such as polyethylene glycol, oils of vegetable origin, hydrogenated naphthalenes, and the like. Formulations for direct administration can include glycerol and other compositions of high viscosity. Other potentially useful parenteral carriers for these therapeutics include ethylene-vinyl acetate copolymer particles, osmotic pumps, implantable infusion systems, and liposomes. Formulations for inhalation administration can contain as excipients, for example, lactose, or can be aqueous solutions containing, for example, polyoxyethylene-9-lauryl ether, glycocholate and deoxycholate, or oily solutions for administration in the form of nasal drops, or as a gel to be applied intranasally. Retention enemas also can be used for rectal delivery.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor ELTM (BASF, Parsippany, N.J.) or phosphate buffered saline (PBS). In all cases, the composition can be sterile and can be fluid to the extent that easy syringability exists. It can be stable under the conditions of manufacture and storage and can be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filter sterilization. Generally, dispersions are prepared by incorporating the active compound into a sterile vehicle which contains a basic dispersion medium and the required other ingredients from those enumerated above. In the case of sterile powders for the preparation of sterile injectable solutions, methods of preparation include vacuum drying and freeze-drying which yields a powder of the active ingredient plus any additional desired ingredient from a previously sterile-filtered solution thereof.

In one embodiment, the therapeutics are prepared with carriers that will protect against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials also can be obtained commercially from Alza Corporation and Nova Pharmaceuticals, Inc. Liposomal suspensions can also be used as pharmaceutically acceptable carriers. These can be prepared according to methods known to those skilled in the art, for example, as described in U.S. Pat. No. 4,522,811. Microsomes and microparticles also can be used.

Oral or parenteral compositions can be formulated in dosage unit form for ease of administration and uniformity of dosage. Dosage unit form refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Proteins and other compositions of the present invention may be formulated within a therapeutic mixture to comprise about 0.0001 to 1.0 milligrams, and/or about 0.001 to 0.1 milligrams, and/or about 0.1 to 1.0 and/or even about 10 milligrams per dose and/or so. Multiple doses can also be administered.

The in vitro activity of the compositions of the present invention can be tested using a variety of known methods including, e.g., a cell-based assay and the activity correlated with the pharmacokinetics and in vivo potency of the composition. Similarly, the in vivo biological activities of the compositions of the present invention can be measured by a variety of known assays conducted in mammalian patient (e.g., a human, non-human primate, or rodent) models, such as, for example, mice and rats.

The compositions of the present invention can further be provided by in vivo expression methods. For example, a nucleic acid encoding an Fc-MDL-1 fusion protein of the present invention can be advantageously provided directly to a patient suffering from a inflammatory disease, or may be provided to a cell ex vivo, followed by administration of the living cell to the patient. In vivo gene therapy methods known in the art include providing purified DNA (e.g. as in a plasmid), providing the DNA in a viral vector, or providing the DNA in a liposome or other vesicle (see, e.g., U.S. Pat. No. 5,827,703 relating to delivery using liposomes, and U.S. Pat. No. 6,281,010 relating to delivery using adenovirus vectors).

Methods for treating disease by implanting a cell that has been modified to express a recombinant protein are also known (e.g., U.S. Pat. No. 5,399,346 relating to methods for introducing a nucleic acid into a primary human cell for introduction into a human).

In vivo expression methods are particularly useful for delivering a protein to targeted tissues or cellular compartment. Thus, compositions and methods of the present invention, encompass the use of gene therapy using a sequence encoding a protein of the present invention for treatment inflammatory diseases. For example, using known methods, a nucleic acid sequence encoding a protein of the present invention can be inserted into an appropriate transcription or expression cassette and introduced into a mammalian host as naked DNA or complexed with an appropriate carrier. Detection and monitoring of the production and activity of the protein can be performed by nucleic acid hybridization, ELISA, Western Blot, and other suitable known methods. It is known that a plurality of tissues can be transformed following systemic administration of transgenes. Also, the expression of exogenous DNA following intravenous injection of a cationic lipid carrier/exogenous DNA complex into a mammalian host has been shown in multiple tissues. Examples of methods for gene therapy delivery include, but are not limited to, those described in U.S. Pat. No. 6,627,615.

Therapeutically-Effective Amount and Dosing Frequency

A pharmaceutical composition of the present invention can be formulated for parenteral or oral administration to humans or other mammals, for example, in therapeutically effective amounts which provide appropriate concentrations of the drug to a target tissue for a time sufficient to induce the desired effect. More specifically, as used herein, “therapeutically effective amount” refers to an amount of a composition of the present invention that results in modulating an MDL-1 activity or function in a cell of a patient. A therapeutically effective amount will vary from one individual to another and will depend upon a number of factors, including the overall physical condition of the patient, severity and the underlying cause of disease.

A therapeutically effective amount of a composition of the present invention may be readily ascertained by one skilled in the art. The effective concentration of a composition of the present invention that is to be delivered for treatment of a disease in a patient will vary depending upon a number of factors, including the final desired dosage of the drug to be administered and the route of administration. The preferred dosage to be administered also is likely to depend on such variables as the type and extent of disease or indication to be treated, the overall health status of the particular patient, the relative biological efficacy of a composition of the present invention to be delivered, the formulation of a composition of the present invention, the presence and types of excipients in the formulation, and the route of administration. In some embodiments, the methods of treatment of the present invention can be provided to a patient using typical dose units deduced from the mammalian studies using non-human primates and rodents. A dosage unit refers to a unitary dose which is capable of being administered to a patient, and which can be readily handled and packed, remaining as a physically and biologically stable unit dose comprising either the composition of the present invention or a mixture of it with solid or liquid pharmaceutical diluents or carriers.

The dosing frequency for a composition of the present invention will vary depending upon the condition being treated. The dosing frequency may be about once or twice per week. The dosing frequency may also be less than about one time per week, for example about once every two weeks (about one time per 14 days), once per month or once every two months. Further, the dosing frequencies actually used may vary somewhat from the frequencies disclosed herein due to variations in responses by different individuals to a composition of the present invention; the term “about” is intended to reflect such variations.

Additionally, the compositions of the present invention can be administered alone or in combination with other molecules known to have a beneficial effect on the particular disease or indication of interest. By way of example only, useful cofactors include symptom-alleviating cofactors, including antiseptics, antibiotics, antiviral and antifungal agents and analgesics and anesthetics.

The invention is further illustrated by the following examples that are not intended in any way to limit the scope of the invention.

### EXAMPLES

### Example 1

### Cloning of a Novel C Type Lectin Receptor in Monocytic Cells

DAP12 is expressed in monocytic cells (Lanier et al. (1998) Nature 391:703-707). The present inventors demonstrate herein that DAP12 interacting receptors may be identified in these cells as well. Using a probabilistic model for the C type lectin superfamily, a template was identified through a proprietary hidden Markov model by searching the Incyte LifeSeq database (a commercially available database which combines and integrates cDNA sequence data with human genome sequence data). A proprietary DNA sequence expression array showed the expression of this template in THP-1, a monocytic cell line.

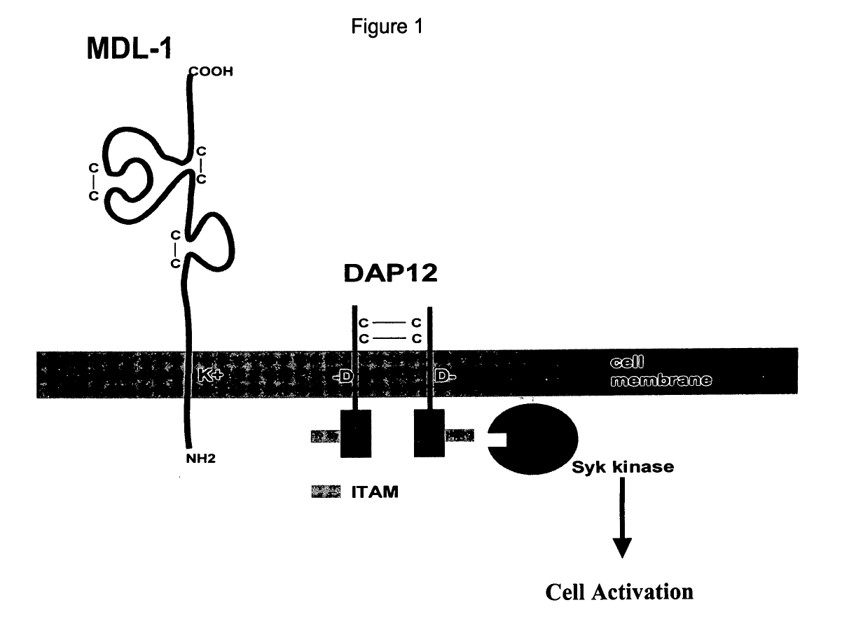

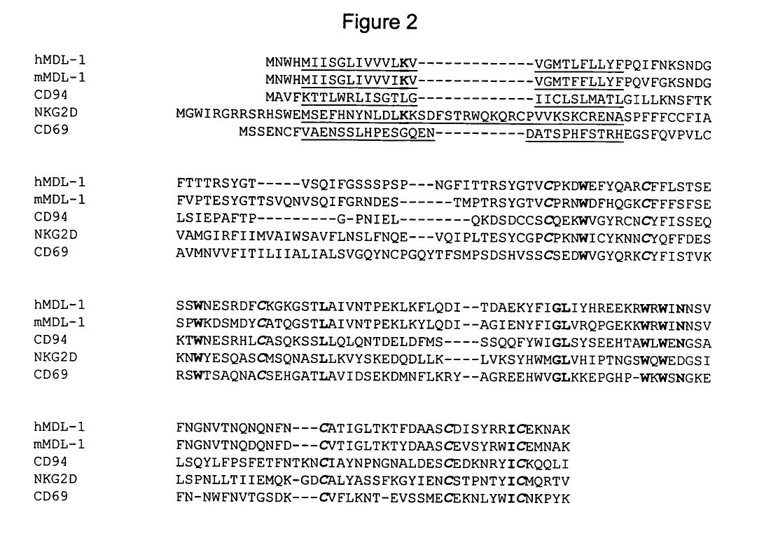

The translational product of the template is the human myeloid receptor designated as myeloid DAP12-associating lectin (hMDL-1). Human MDL-1 is a type II membrane protein with a single TM domain possessing a positively charged K residue (FIGS. 1 and 2), which is essential for interacting with DAP12. The predicted cytoplasmic domain is short with only four amino acid residues and lacks known signaling motifs. For the extracellular C type lectin domain, it shares important residues known to be conserved in the C type lectin superfamily members e.g., as shown in FIG. 2. There are two known N-glycosylation sites in the extracellular domain. Database searching identified a murine homolog of MDL-1 (mMDL-1), which shares 70% identity with the human protein (FIG. 2).

Based on the human MDL-1 amino acid sequence a calculated molecular weight (MW) for human MDL-1 is 21.5 kilodalton (kDa). Since MDL-1 is a glycosylated protein, the apparent MW for human MDL-1 in SDS PAGE is about 40 kDa. Based on the mouse MDL-1 amino acid sequence mouse, a calculated MW for mouse MDL-1 is 21.7 kDa.

### Example 2

### Expression Pattern of MDL-1 and Its Association with Inflammation

The nucleotide sequence of human MDL-1 was used to search the Incyte LifeSeq database. The cells and tissues which serve as mRNA sources for MDL-1 indicate that the gene may be expressed in normal lungs, peripheral blood mononuclear cells and macrophages, monocytes stimulated with LPS, macrophages and lymphocytes after mixed leukocyte activation, and THP-1 cells stimulated with PMA/LPS. Human MDL-1 was also detected in brain pons and choroid plexus from a patient with Huntington's disease, and brain hippocampus from a patient with multiple microinfarcts (using the Incyte LifeSeq database). These results indicate that hMDL-1 may be involved in inflammation in the CNS. The expression pattern of hMDL-1 in normal tissues was confirmed by Northern blot (data not shown).

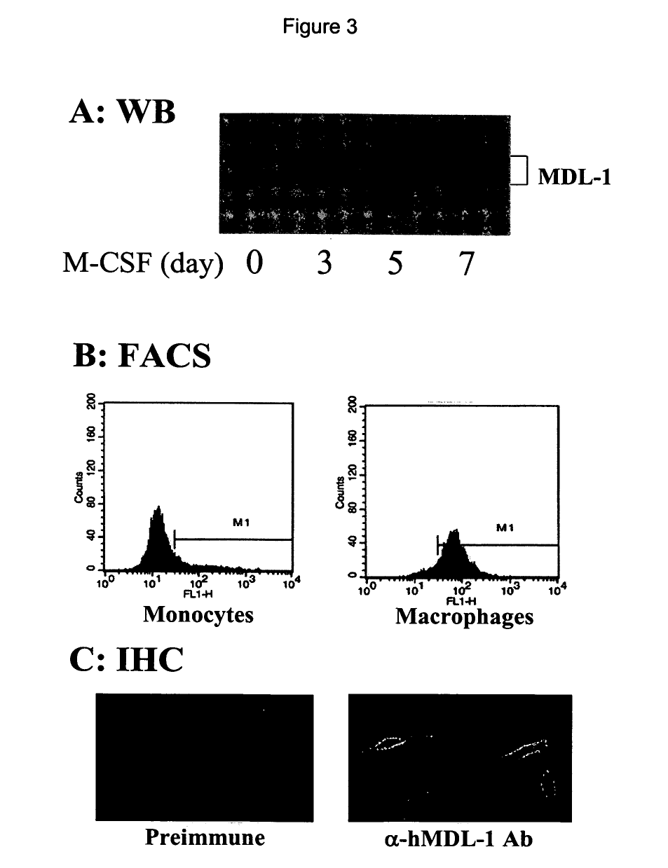

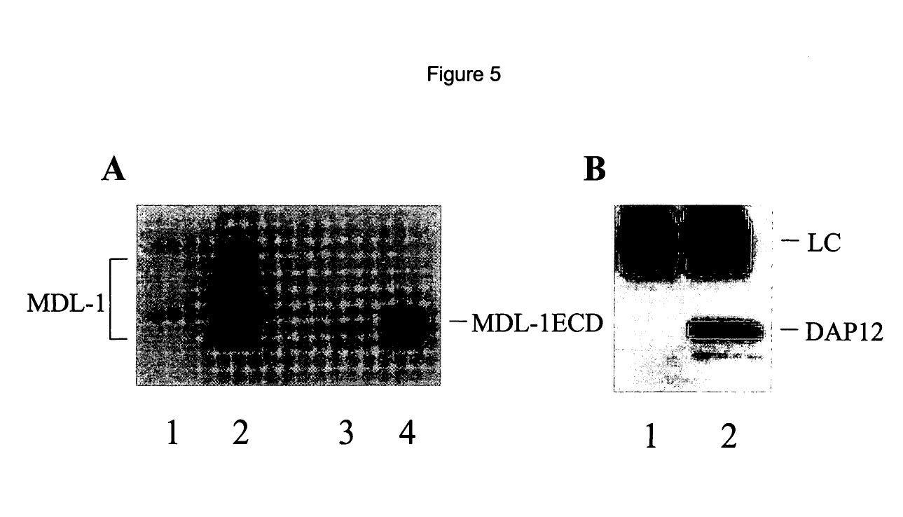

Rabbit antiserum was raised against baculovirus-insect cell expressed extracellular domain (ECD) of human MDL-1 protein. The rabbit anti-human MDL-1 (α-hMDL-1) polyclonal antibodies detected MDL-1 protein in Western blot (WB, FIG. 3A), immunoprecipitation (IP, FIG. 5B), fluorescence-activated cell sorting (FACS, FIG. 3B), and immunohistochemistry (IHC) assays (FIG. 3C). Using rabbit α-hMDL-1 antibody, the present inventors found no detectable surface expression of hMDL-1 in CD14+monocytes. In contrast, M-CSF (or GM-CSF) differentiated macrophages showed remarkable surface expression (FIG. 3).

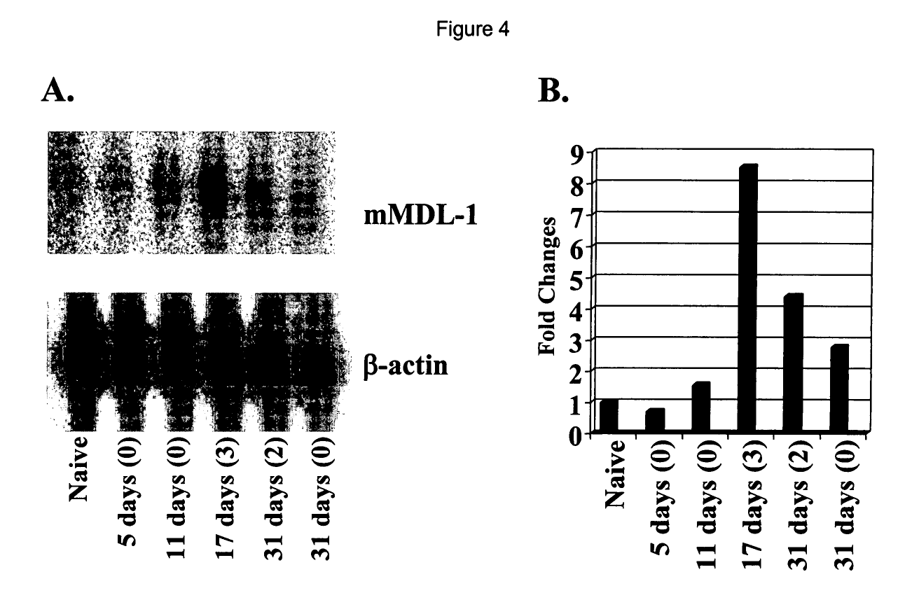

In addition, Northern blots generated from RNA isolated over the disease course in a chronic mouse model of EAE showed a direct correlation of mMDL-1 expression and disease score (FIG. 4). Specifically, mMDL-1 expression increases above background levels in naïve mice at the onset of clinical disease (day 11), and reaches maximum expression at the peak of the disease on day 17 (FIG. 4). The overall increase of mMDL-1 specific transcripts is 8-fold above background on day 17.

Taken together, these data suggest that mMDL-1 may be involved in inflammatory process and in the pathogenesis of inflammatory disease, e.g., EAE.

### Example 3

### Function of MDL-1 Through Interaction with DAP12

When DAP12 is expressed in the absence of MDL-1, the membrane bound adaptor protein (DAP12), is mainly found intracellularly. In contrast when DAP12 is coexpressed with MDL-1, DAP12 is translocated to the cell surface (e.g., Bakker et al. (1999) Proc. Natl. Acad. Sci. USA 96:9792-9796). In view of this data, it has been proposed that MDL-1 directly interacts with DAP12 through an electrostatic attraction between charged amino acid residues in their transmembrane domains, a positively charged K in MDL-1 and a negatively charged D in DAP12 (e.g., Lanier and Bakker (2000) Immunol. Today 12:611-614). However, prior to the data provided herein by the present inventors, evidence for a direct physical association between MDL-1 and DAP12 had not been previously demonstrated. Thus, the present inventors carried out experiments to address this issue. Immunoprecipitation of cellular lysates of human MDL-1 and DAP12 (hDAP12) expressing cells with rabbit α-hMDL-1 antibodies followed by immunoblotting with anti-hDAP12 antibodies clearly demonstrated a physical interaction between human MDL-1 and DAP12 (FIG. 5).

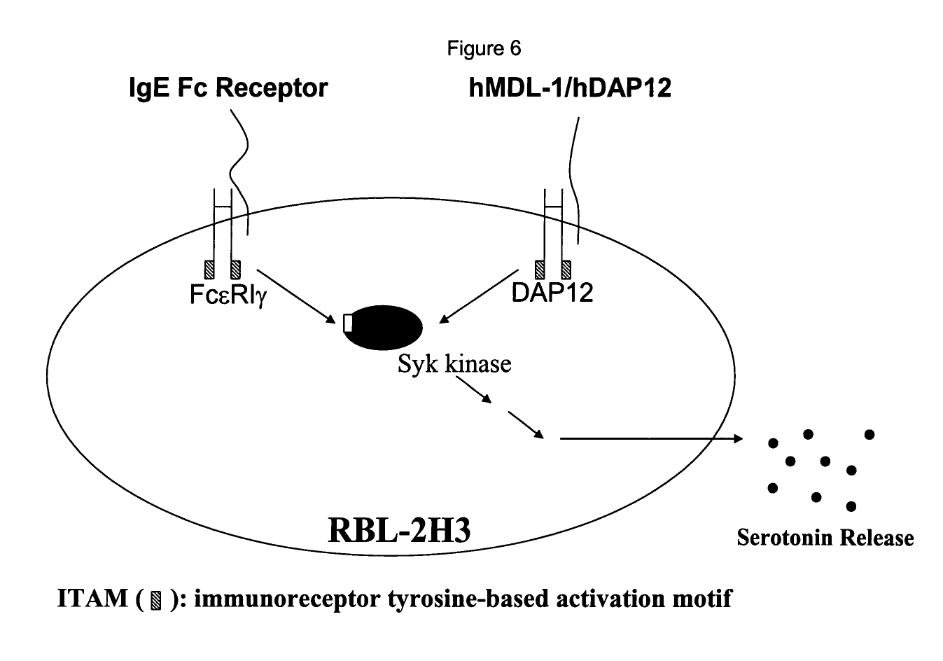

Furthermore, a rat basophilic leukemia cell line, RBL-2H3 was used to demonstrate a functional coupling between hMDL-1 and hDAP12. Engagement of cell surface IgE receptor (FcεRI) complexes triggers RBL degranulation through activation of Syk kinase pathways, which then leads to the release of serotonin (e.g., Tomasello et al. 2000). The present inventors generated stable RBL transfectants that express both human MDL-1 and DAP12 (RBL-hMDL-1/hDAP12) (FIG. 6).

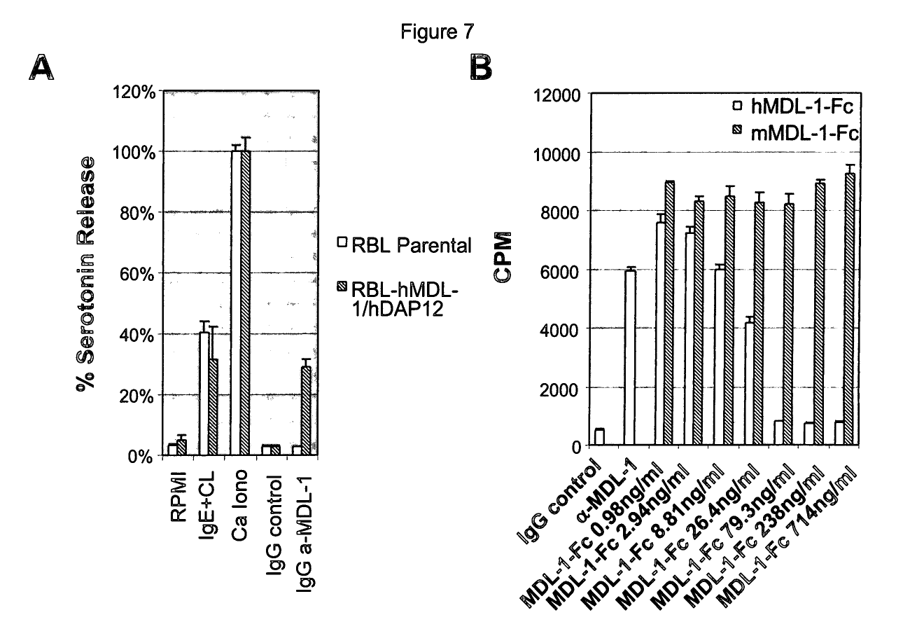

FACS analysis indicated the expression of both hMDL-1 and hDAP12 on the surface of RBL transfectants (Data not shown). The transfectants released serotonin when stimulated with rabbit α-hMDL-1 antibodies in contrast to parental RBL cells, which were insensitive to this treatment (FIG. 7A). The extracellular domain of a human and a mouse MDL-1 were each fused with the Fc portion of human immunoglobulin IgG1 (hMDL-1-Fc and mMDL-1-Fc). Baculovirus expressed hMDL-1-Fc fusion protein, but not mMDL-1-Fc, specifically blocked serotonin release in a dose dependent fashion (FIG. 7B). An RBL transfectant with only hMDL-1 expressed on the cell surface was not capable of releasing serotonin when activated with α-hMDL-1 antibodies (data not shown). The serotonin release assay in RBL transfectants thus clearly demonstrates that the function of hMDL-1 is dependent on its association with hDAP12.

In addition to the RBL transfectants described, we have established a second functional assay to measure the association and receptor-specific signaling of MDL-1 and DAP-12. A stable line, CD3zH912Z1, expressing chimeric protein encoding human MDL-1 extracellular domain (hMDL-1ECD) fused at the C-terminus of intracellular CD3ζ and FLAG-hDAP12 was made in the BWZ.36 cell line and is described herein below.

### Example 4

### Chemokine and Cytokine Release in MDL-1 Activated Macrophages

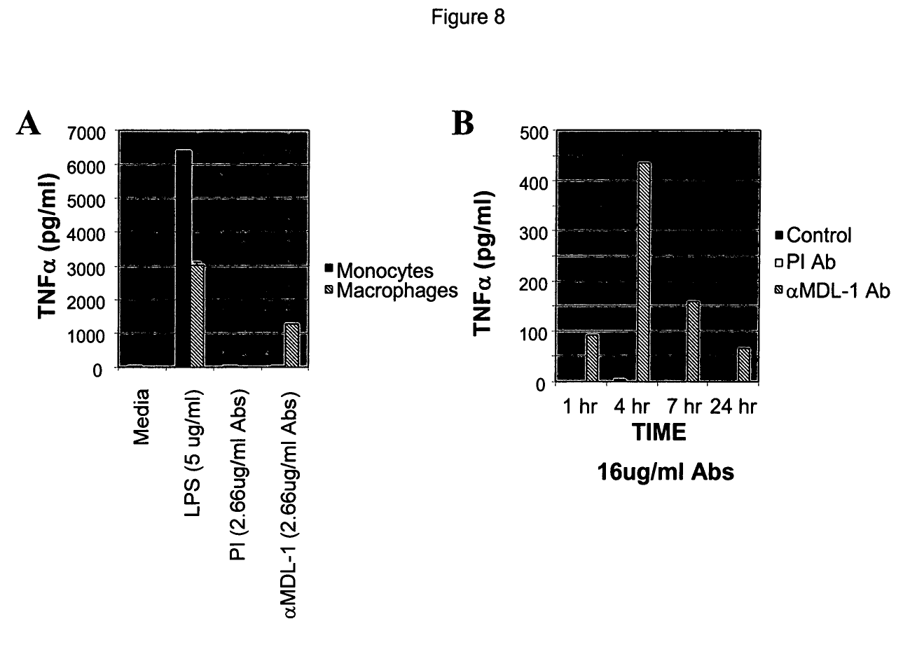

Results from the serotonin release assay in RBL-MDL-1/DAP12 double transfectants suggest that rabbit anti-human MDL-1 polyclonal antibodies function as activating antibodies (FIG. 7). To examine whether the activation of MDL-1 protein can trigger the secretion of chemokines and cytokines, the α-hMDL-1 antibodies were incubated with human macrophages expressing MDL-1 on the cell surface after GM-CSF differentiation. Cross-linking of hMDL-1 in macrophages induced secretion of a variety of pro-inflammatory chemokines and cytokines. FIG. 8A shows the increased production of TNFα in macrophages, but not in monocytes after activation with α-hMDL-1 antibodies. The production of TNFα appears to peak after 3 hours (FIG. 8B). There was also a similar increase in the production of MCP-1, MIP-1α, and IL-6 in α-hMDL-1 antibody activated macrophages, but not IL-8, IL-12, and RANTES (data not shown). The release of chemokine and cytokine in α-hMDL-1 antibody activated macrophages was specifically blocked with incubation of baculovirus expressed hMDL-1-Fc fusion protein (data not shown). The release of these pro-inflammatory chemokines and cytokines upon activation of hMDL-1 strongly suggests the involvement of MDL-1 in inflammatory processes, which play an important role in the pathogenesis of diseases such as MS, IBD, and arthritis.

### Example 5

### Generation of Rabbit α-Mouse MDL-1 Antibodies

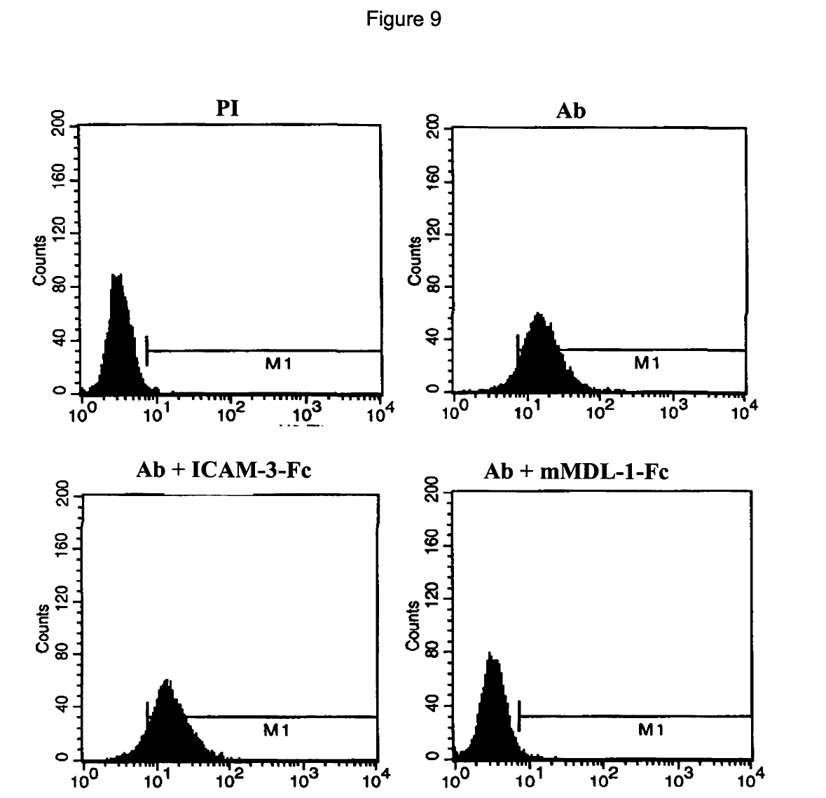

The present studies with human MDL-1 show that polyclonal antibodies raised against this protein are useful tools to characterize and validate MDL-1 and DAP12 stable transfectants and can be used as MDL-1 surrogate ligands in functional assays. Based on these data, baculovirus expressed mouse MDL-1-ECD was used as an antigen to generate rabbit anti-mouse MDL-1 (α-mMDL-1) polyclonal antibodies. Two rabbit α-mMDL-1 antibodies were characterized and validated using 293-mMDL-1/mDAP12 stable transfectant. The rabbit α-mMDL-1 antibody specifically detected surface expression of mMDL-1 by FACS analysis (FIG. 9). The binding of α-mMDL-1 antibody was disrupted in the presence of baculovirus expressed mMDL-1-Fc, but not ICAM-1-Fc fusion (FIG. 9).

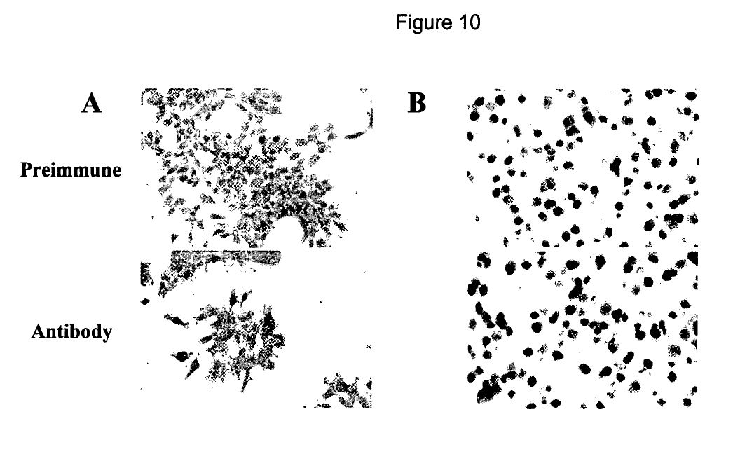

IHC (immunohistochemistry) staining protocols with rabbit α-mMDL-1 antibodies were established. FIG. 10 clearly shows the surface staining of MDL-1 in 293-mMDL-1/mDAP12 transfectant in cell culture (FIG. 10A) and paraffin-embedded transfected cells (FIG. 10B). As described herein, the α-mMDL-1 antibodies of the present invention can be used for IHC staining and FACS analysis.

### Example 6

### A Cellular Assay to Identify the MDL-1 Ligand by Function

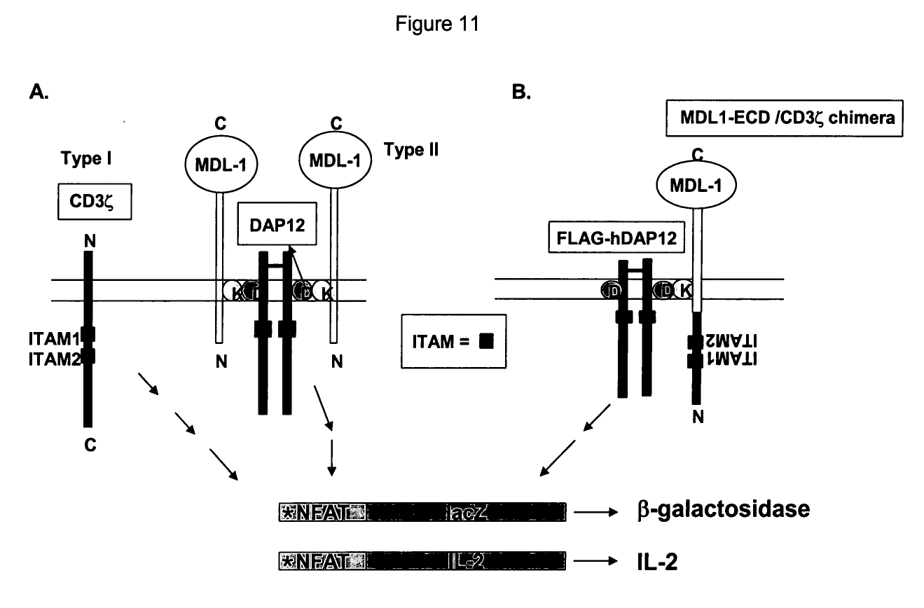

A second functional assay was established by the present inventors to measure the association and receptor-specific signaling of MDL-1 and DAP-12 by transfecting the BWZ.36 cell line. BWZ.36 is a clonal murine thymoma line (TCRα⁻β⁻) expressing the reporter gene β-galactosidase under the control of a minimal IL-2 promoter stably integrated into the genome (e.g., Sanderson and Shastri (1994) Int. IMMunol. 6:369-76). The present inventors generated stable lines were generated by transfection of BWZ with hMDL-1 and hDAP12 encoding cDNA. Several strategies were tested to derive the most sensitive reporter cell. Examples of these strategies include, but are not limited, to those illustrated in FIG. 11.

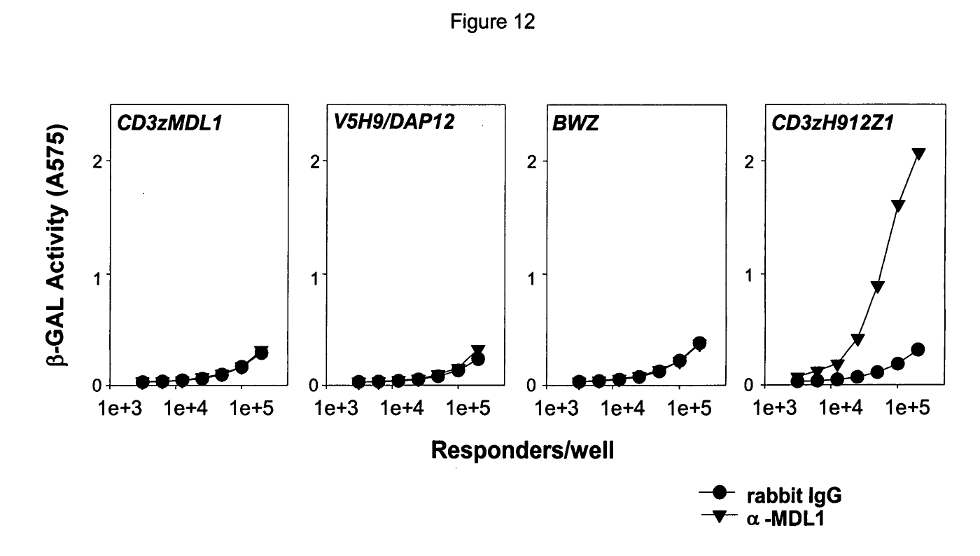

Three types of transfectants were made by the present inventors. One was transfected with a full-length MDL-1 and DAP12 (V5H9/DAP12); a second was transfected with an MDL-1CD3ζ chimera alone (CD3zMDL1; e.g., Weiss et al. (1994) Immunol. Rev. 163:19-34); and a third was transfected with both the MDL-1CD3ζ chimera and DAP12 (CD3zH912Z1). Stimulation of reporter cells with α-hMDL-1 antibodies resulted in β-galactosidase expression only in the CD3zH912Z1 transfectants while the remaining lines were similar to the parental line BWZ.36 (see FIG. 12). Both MDL-1CD3ζ chimera and DAP12 were detected by FACS analysis (data not shown).

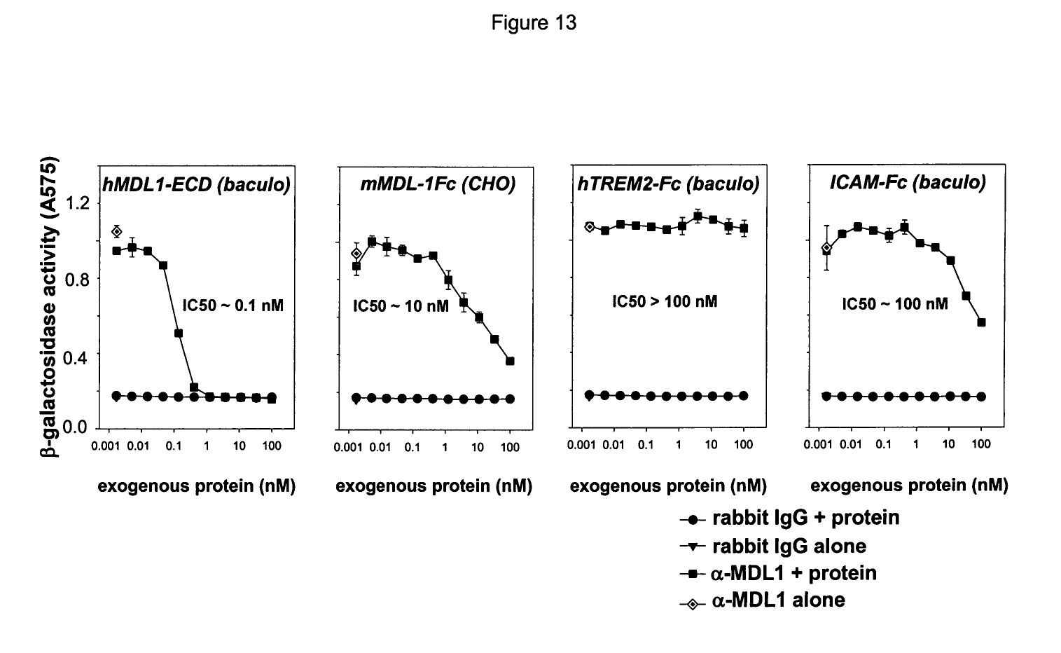

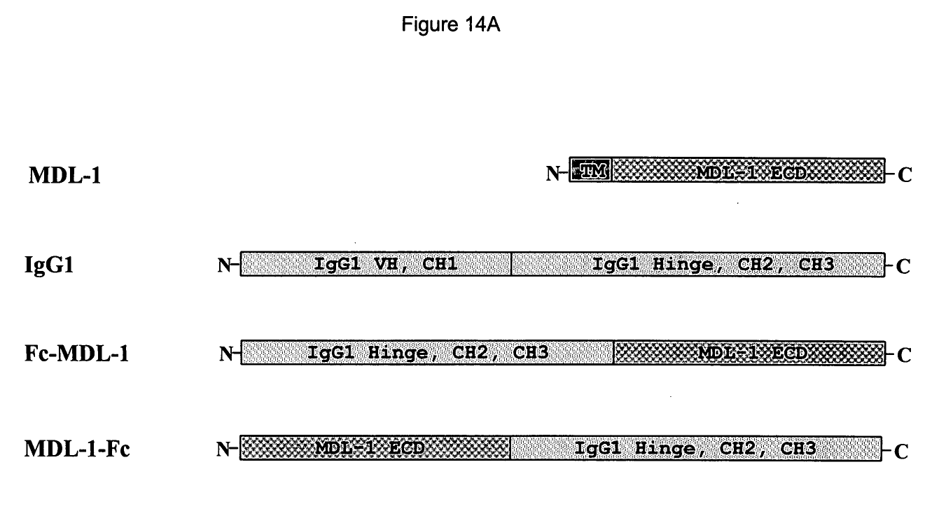

Expression of the reporter gene was specific as it could be inhibited by soluble hMDL-1ECD protein added exogenously, but not by addition of soluble TREM2-Fc fusion protein. Soluble ICAM-Fc fusion protein however was partially capable of inhibiting the reporter cell, most likely due to an effect on adhesion rather than a direct interaction with the MDL-1 receptor (FIG. 13). Interestingly, the murine MDL-1-Fc protein inhibited signaling in the CD3zH912Z1 line although the IC50 for this inhibition was approximately 10 nM while that of the human MDL-1ECD protein was 0.1 nM.

CD3zH912Z1 as well as other lines generated with the MDL-1CD3ζ chimera and DAP12 were able to signal upon ligation with α-MDL-1 antibody. Additionally, ligation of DAP12 directly via a FLAG epitope at the N-terminus of that protein with α-FLAG antibody resulted in equivalent levels of β-galactosidase activity (data not shown). These results demonstrate the co-localization of DAP12 and the MDL-1CD3ζ chimera at the plasma membrane by function.

### Example 7

### Generation and Characterization of Mammalian Expressed Mouse and Human MDL-1-Fc Fusion Proteins

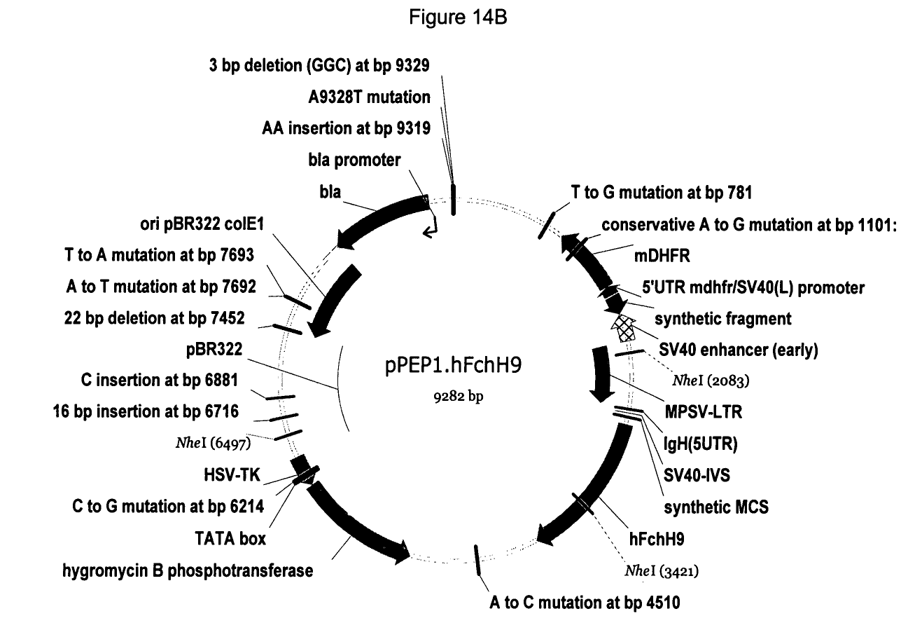

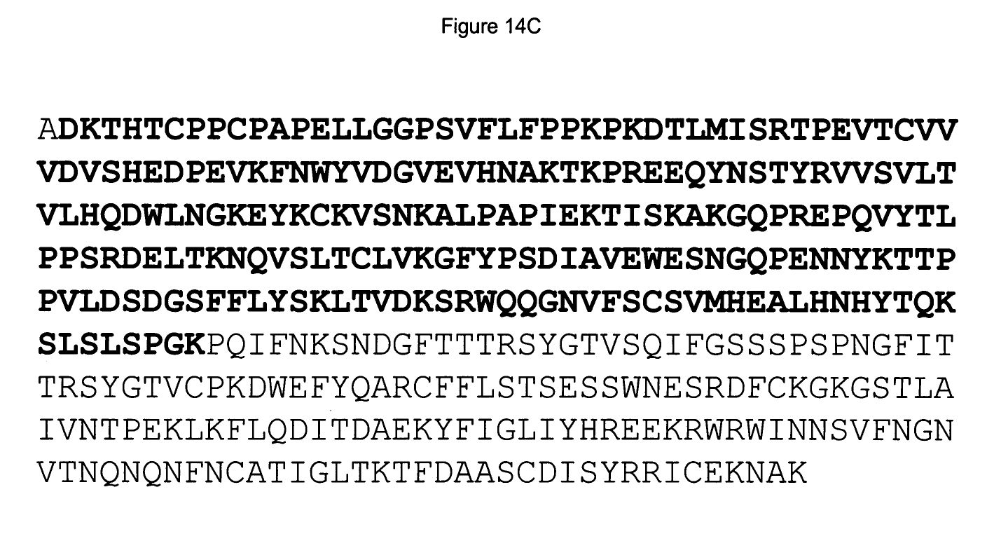

To generate a mouse MDL-1 fusion protein, the present inventors fused the extracellular domain of mouse MDL-1 to the hinge, CH2, and CH3 region of human immunoglobulin IgG1 (mMDL-1-Fc) as illustrated in FIG. 14A. Similarly, to generate a human MDL-1 fusion protein, the present inventors fused the extracellular domain of human MDL-1 to the hinge, CH2, and CH3 region of human immunoglobulin IgG1 (mMDL-1-Fc) as illustrated in FIGS. 14A-C. The MDL-1 protein is a type II membrane protein and its N-terminus is located intracellularly. Therefore, in the fusion protein, the C-terminus of MDL-1 is directly connected to Fc instead of being free, and there is a hinge region between the MDL-1-ECD and Fc.

As described herein, both a human MDL-1-Fc (hMDL-1-Fc) illustrated in FIGS. 14A-C, and a mouse MDL-1-Fc (mMDL-1-Fc) protein were successfully generated in a baculovirus-insect cell expression system. Both the human (FIGS. 14A-C) and the mouse (FIG. 14A) MDL-1-Fc fusion proteins are capable of competing with the natural MDL-1 protein in FACS analysis and several functional assays, which indicates that the human and the mouse MDL-1-Fc fusion protein each fold properly (FIG. 7, 9, 13 and data not shown).

MDL-1 protein is a N- and O-linked glycoprotein (FIG. 2 and data not shown). It has been reported that insect cells are not able to generate eukaryotic glycoproteins with complex N-linked glycans. N-linked glycans are mainly high-mannose-type glycans in insect cells (e.g., Jarvis et al. (1998) Curr. Opin. Biotechnol. 9:528-533). Recently crystal structural data in combination with binding studies have revealed that DC-SIGN and DC-SIGNR, which are cell surface receptors expressed on dendritic cells, endothelium of liver sinusoids, and lymph nodes, selectively recognize high-mannose oligosaccharides (e.g., Feinberg et al. (2001) Science 294:2163-2166). Further, human dendritic cells non-specifically associate with baculovirus expressed proteins, most likely due to insect-specific glycosylation. In order to eliminate the potential in vivo binding of baculovirus-insect cell expressed glycoprotein, the present inventors have generated a mammalian expression system to produce mouse MDL-1-Fc fusion protein for animal efficacy studies.

MDL-1 is a type II membrane receptor and its C-terminus is located extracellularly. When MDL-1 extracellular domain is fused to the Fc region of human IgG1, there are two possible orientations (FIGS. 14A-C). The orientation of the fusion protein may play an essential role in determining the PK profile, efficacy, and immunogenicity of the fusion protein.

Once the relative orientation of the hFc and hMDL-1 fusion protein is chosen, the cDNA encoding the fusion protein can be expressed using an expression vector e.g., pPEP1, with MPSV promoter, hygromycin selection marker, and DHFR for MTX amplification (FIGS. 14A-C).

As mentioned, the mouse MDL-1 fusion protein has a two amino acid linker, AS, between mMDL-1 and mFc domains. Thus, a potential modification to the fusion protein can be to delete the linker. This fusion protein can be compared with mMDL-1 fusion protein with AS linker e.g., in a mouse colitis efficacy study.

The hMDL-1 fusion protein can be produced in large quantities in various expression systems, e.g., dihydrofolate reductase-deficient Chinese hamster ovary (CHO) cell line DXB11 or DG44, commonly used as a host cell for production of recombinant proteins. As an example, the expression construct can be transfected into both DXB11 and DG44 CHO cell lines and co-selected with hygromycin and methotrexate. The population can be subjected to multi-rounds of methotrexate amplification and then cloned. The established cell lines can then be checked for expression levels. The expression level of cell lines would be preferably greater than 20 pg/cell/day. The hMDL-1 fusion protein expressing CHO cell lines can be used for large-scale production of the fusion protein for use in further validation, pharmacokinetic and safety studies.

Rabbit anti-human MDL-1 polyclonal antibodies have been used for the ELISA assays to detect the expression level of the fusion protein. Additionally, anti-human MDL-1 monoclonal antibody can be generated for use in FACS analysis and ELISA assays.

The fusion product can be analyzed and validated using known assays to determine its physico-chemical properties, purity, dimer status, endotoxin level, and activity. Examples of such assays include, but are not limited to:

UV spectroscopy/ELISA assay—concentration

Size exclusion chromatography—purity/molecular size

N-terminal sequencing—identity

SDS-PAGE—identity/purity

Western Blot—identity

HPLC-SEC—purity

FFF-LALS—molecular size

Endotoxin—purity

Stability studies with or without serum—stability.

Examples of functional assays can be include, but are not limited to:

Serotonin release assay—cell assay for activity

Human macrophage cytokine release assay—cell assay for activity.

The fusion product can be further characterized for its PK profile and immunogenicity. The present inventors have established and validatd an MDL-1 specific ELISA, to determine the blood concentration of the fusion protein. Typical PK parameters such as half-life, AUC, plasma clearance, and volume of distribution can be determined using known assays.

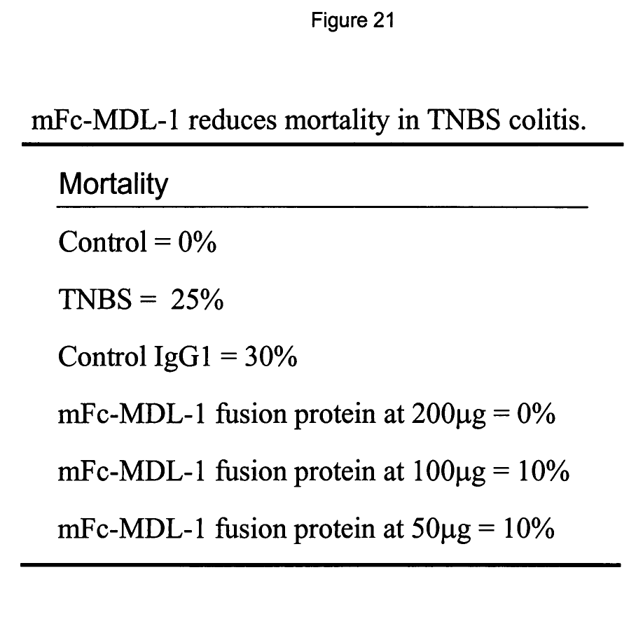

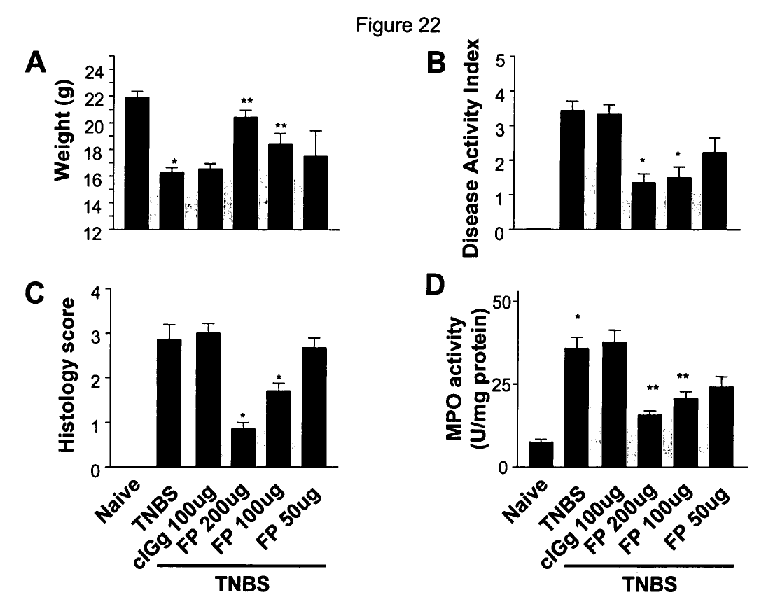

It has been reported that IBD patients have elevated production of pro-inflammatory cytokines such as IL-12, TNF-α, and IFN-γ. TNF-α is increased in the stool, intestinal mucosa, and blood of patients with Crohn's disease (Sandborn 2005). Anti-TNF-α therapy has been utilized clinically with success to treat patients with Crohn's disease. As demonstrated herein, production of TNF-α is also elevated in the blood and mucosa of colitis mouse. The mFc-mMDL-1 fusion protein is capable of reducing the production of TNF-α and ameliorating the disease (FIGS. 21 and 22). Therefore TNF-α can be used as a biomarker for the therapeutic effects of MDL-1 fusion protein in clinical trials. Other cytokine biomarkers will be identified using commercially available human cytokine antibody microarrays.

### Example 8

### Characterization of mMDL-1-Fc Fusion Protein In Vitro

In order to assess the therapeutic value of the mMDL-1-Fc fusion protein, it can first be tested for the inhibition of the biological activity of MDL-1 in in vitro assays. The criteria used to test the effectiveness of the mMDL-1-Fc can be two-fold e.g., based first on its ability to block the binding of α-mMDL-1 antibodies to cell surface MDL-1 receptor and based second, on its ability to block the activating function of α-mMDL-1 antibodies, including serotonin release assay in RBL transfectants, β-galactosidase assay in BWZ.36 transfectants, or chemokine and cytokine release assay in macrophages (FIG. 14). Purified mMDL-1-Fc fusion protein can be used as a competitor to block the action of α-mMDL-1 antibodies in FACS and activation assays while normal human IgG1 protein can be used as a negative control to evaluate the specific activity of mMDL-1-Fc.

### Example 9

### In Vivo PK Profile of mFc-mMDL-1 Fusion Protein

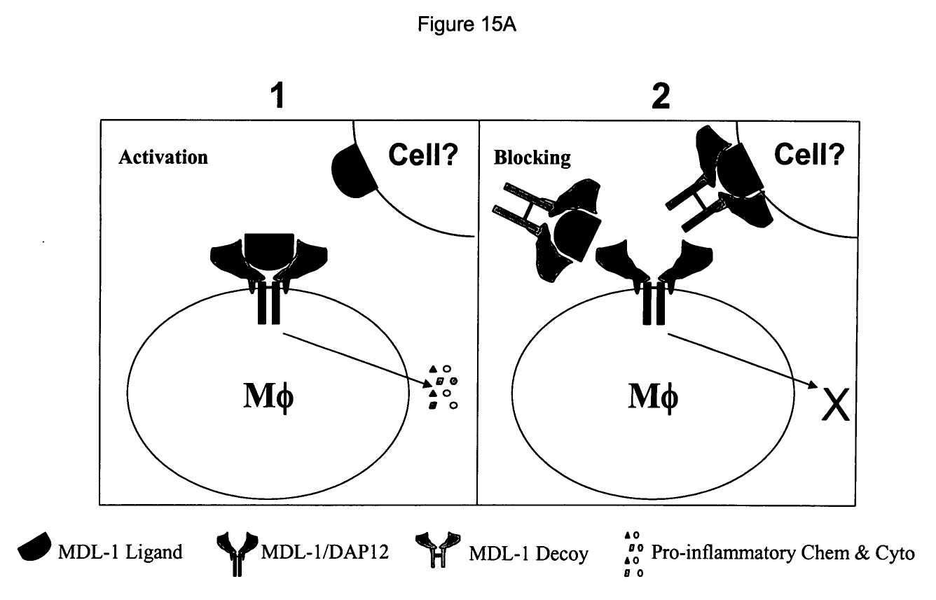

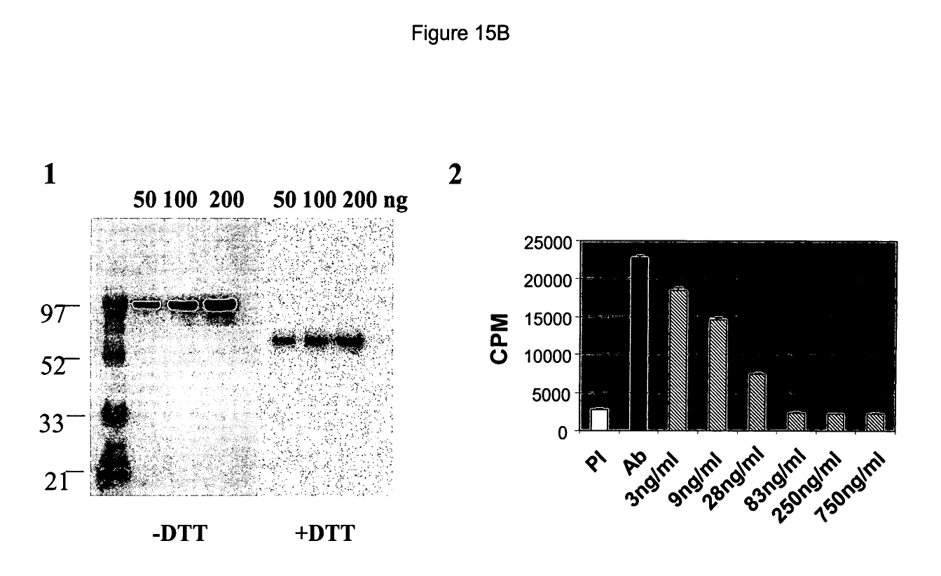

To test the role of MDL-1 in the pathogenesis of inflammatory diseases, a MDL-1 decoy protein was designed to bind the MDL-1 ligand and therefore, modulate, e.g., antagonize, the ability of MDL-1 ligand to activate MDL-1 signaling pathway. One example of such modulation includes, but is not limited to that illustrated in FIG. 15A, wherein one modes of action of the MDL-1 fusion protein is to bind to a cognate ligand of MDL-1 and thereby prevent MDL-1 from binding and activating the MDL-1 signaling pathway. However, other modes of action are contemplated and not limited to the example illustrated in FIG. 15A.

As described herein, MDL-1 decoy protein is a fusion protein with mouse MDL-1 extracellular domain fused to the C-terminus of the hinge, CH2 and CH3 regions of mouse IgG1. It has been reported that Fc improves the stability and generates a dimerized form of fusion protein. There are an additional two amino acids, alanine and serine (AS), between mFc and mMDL-1 in the fusion protein, mFC-mMDL-1. The fusion protein, mFc-mMDL-1, was expressed in HEK293 cells and purified by affinity chromatography with high purity and biologic activity as shown in FIG. 15B.

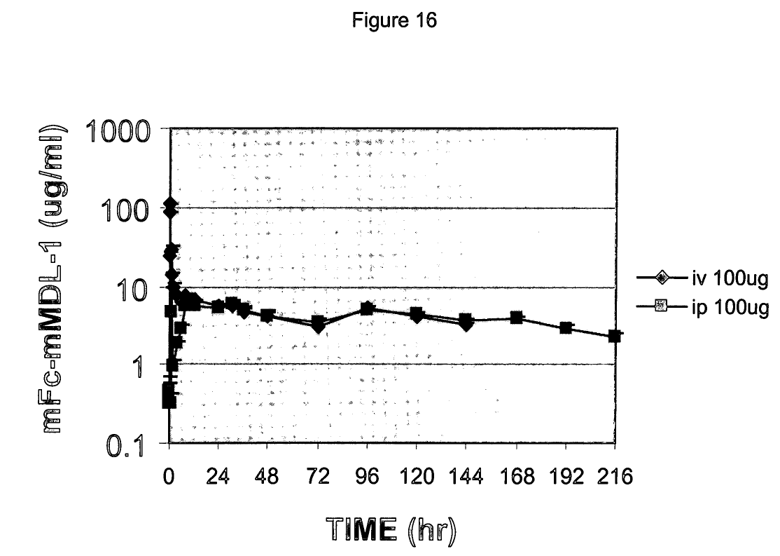

In vivo pharmacokinetics of mFc-mMDL-1 fusion protein was evaluated in mouse following a single dose intravenous and intraperitoneal administration. Table 1 shows the PK parameters for the mFc-mMDL-1 fusion protein. PK study demonstrated that concentration of the fusion protein reached same level after 12 hr of administration in both routes (FIG. 16). mFc-mMDL-1 fusion protein keeps at relatively high blood concentration, ˜4 ug/ml, for at least 7 days. The bioavailability of the fusion protein after i.p. administration is approximately 80% in the mouse.

TABLE 1
Pharmacokinetic parameters for mFc-mMDL-1
fusion protein in SJL mice.

| Route/Dose | T_{1/2} | CI total | Tₘₐₓ | Cₘₐₓ | Vss | AUC |
|---|---|---|---|---|---|---|
| (mg/mouse) | (h) | (ml/h) | (h) | (mg/ml) | (ml) | (h*mg/ml) |
| IV Injection | 50 | 0.13 | n.a. | n.a. | 10 | 701 |
| (100 mg/mouse) | | | | | | |
| IP Injection | 206 | n.a. | 30 | 6.03 | n.a. | 866 |
| (100 mg/mouse) | | | | | | |

n.a. = not applicable

### Example 10

### Immunogenicity of mFc-mMDL-1 Fusion Protein

As described herein, mFc-mMDL-1 was evaluated in murine chronic and acute EAE models. SJL mice were used in both EAE models. mFc-mMDL-1 fusion protein was repeatedly administrated at 100 ug/mouse and 200 ug/mouse by intraperitoneal route. In a 23 day study of acute EAE model, fusion protein was injected on day 2, 6, 9, 13, 16, and 20. At the end of the study, there was no detectable anti-mMDL-1 antibodies in the sera of SJL mice. In a 28 day study of chronic EAE model, a total of 9 injections were given on day 1, 3, 5, 8, 10, 12, 15, 17, and 22. No anti-mMDL-1 antibodies were detected on day 28^{th}. The results of these studies indicate that mFc-mMDL-1 fusion protein does not appear to have any problem of immunogenicity with short-term use.

### Example 11

### MDL-1 Expression in IBD Tissues

A. MDL-1 Expression in Human IBD Tissues

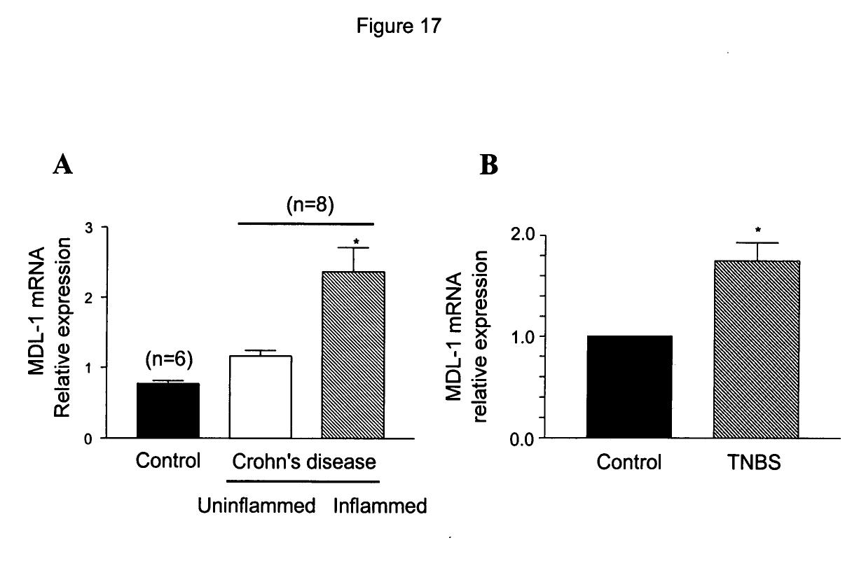

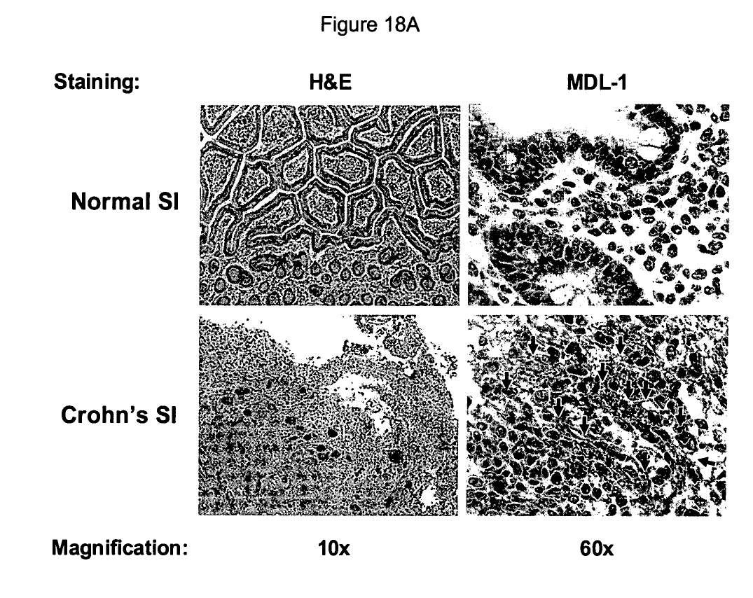

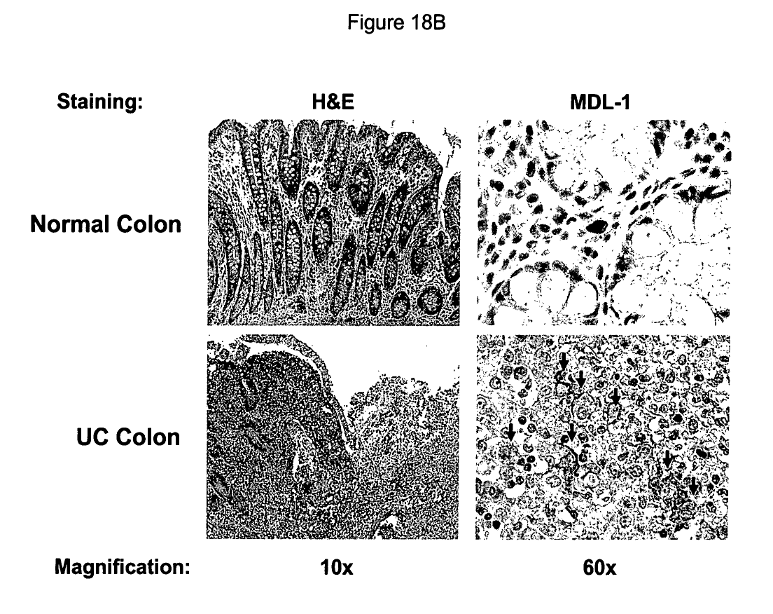

Preliminary study has shown that the expression of human MDL-1 is significantly increased in the inflamed tissues of patients with Crohn's disease (FIG. 17A). Immunohistochemical (IHC) staining further confirmed the upregulation of MDL-1 protein in the ulcer bed of Crohn's disease and ulcerative colitis (FIGS. 18A-B).

B. MDL-1 Expression in Mouse Colitis Tissues

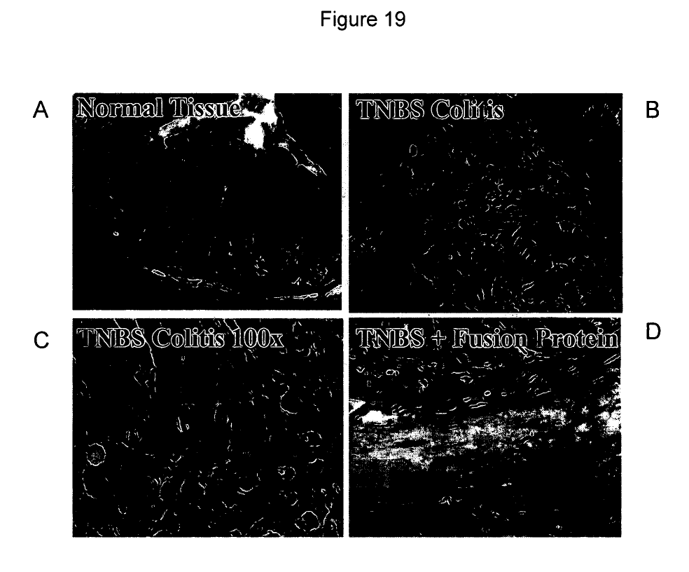

Similar upregulated expression of mouse MDL-1 has also been demonstrated in mouse colitis tissues as shown in FIGS. 17B and 19.

### Example 12

### MDL-1 Fusion Protein in Murine Colitis Models

To determine if MDL-1 plays a pathogenic role in IBD, we tested the efficacy of mFc-mMDL-1 fusion protein in mouse IBD models. The models selected are trinitrobenzene sulfonic acid (TNBS)-induced and dextran sulfate sodium (DSS)-induced colitis. These two colitis models resemble human Crohn's disease on both histological and immunological levels and are widely used to evaluate new anti-inflammatory therapies (e.g., Stadnicki and Colman (2003) Arch. Immunol. Ther. Exp. 51:149-155).

A. TNBS-Colitis

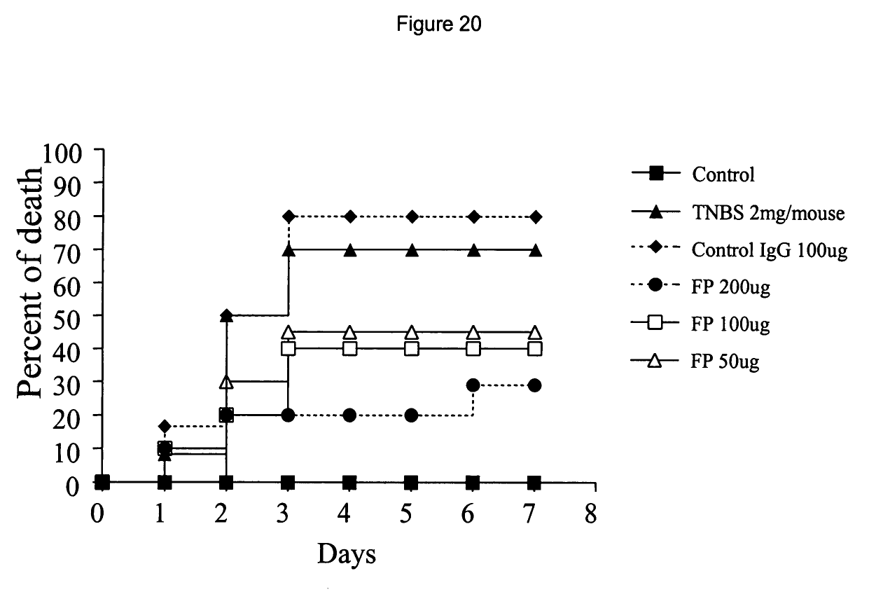

The usual amount of TNBS used to induce mouse colitis is from 0.5 mg-2.5 mg/mouse. We used 2 mg/mouse TNBS in our study to induce severe colitis. As shown in FIG. 20, mFc-mMDL-1 fusion protein significantly reduced mortality in a dose dependent fashion. The mortality rate was 70% in the control group, and 30% with the highest dose of mFc-mMDL-1 fusion protein.

Furthermore, anti-inflammatory activity was assessed in a mild colitis model (with 0.5 mg/mouse TNBS). Similar to the above study, MDL-1 fusion protein reduced the mortality (FIG. 21). In this study, mFc-mMDL-1 fusion protein also attenuated TNBS induced colitis in the measurement of weight loss, disease index, disease histology score, and MPO activity (FIG. 22).

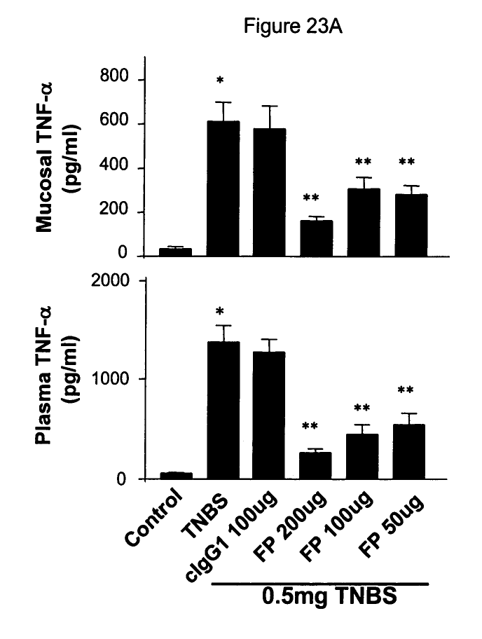

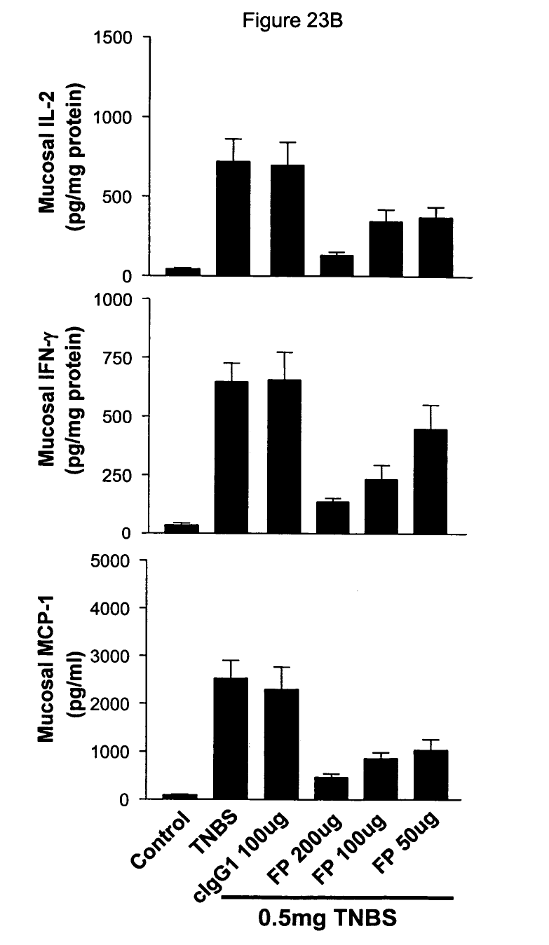

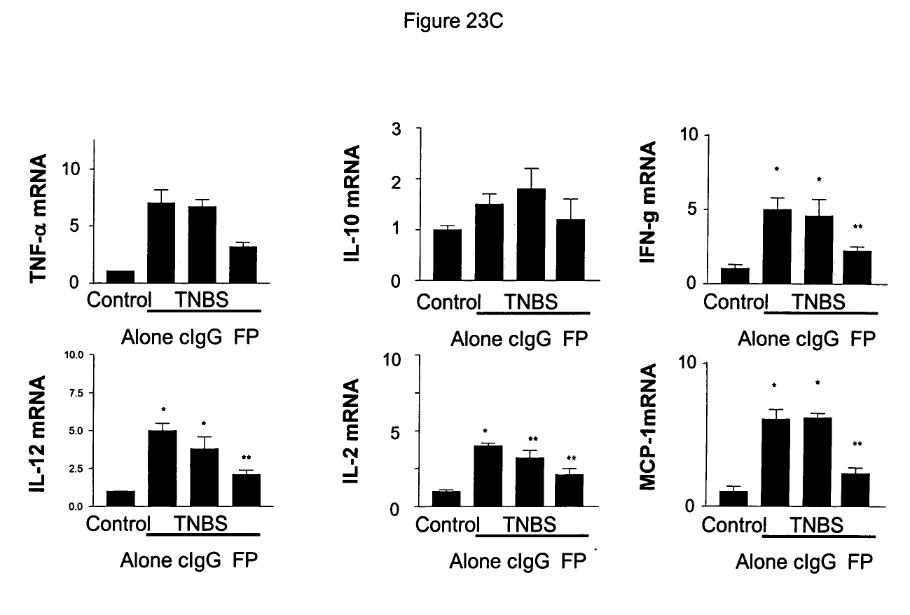

Pro-inflammatory chemokines and cytokines play an important role in the pathogenesis of IBD. In mouse TNBS-colitis, the level of pro-inflammatory mediators, such as MCP-1, TNF-α, IFN-γ, IL-2, and IL-12, were dramatically reduced after the administration of MDL-1 fusion protein (FIGS. 23A, B, and C).

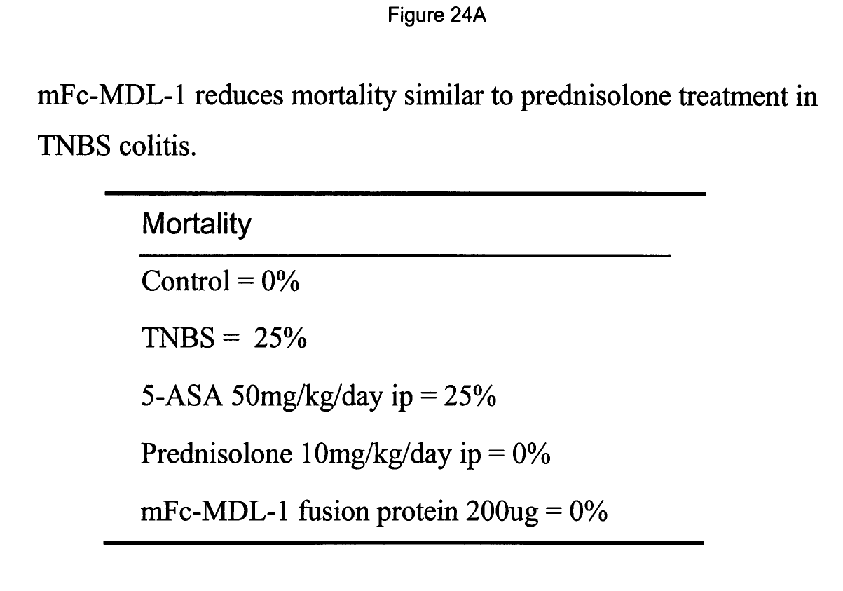

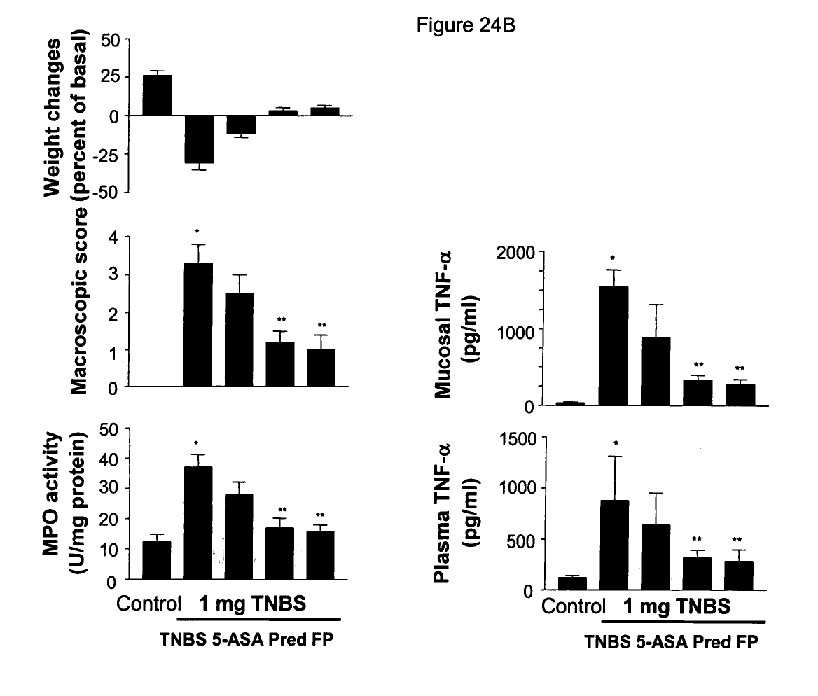

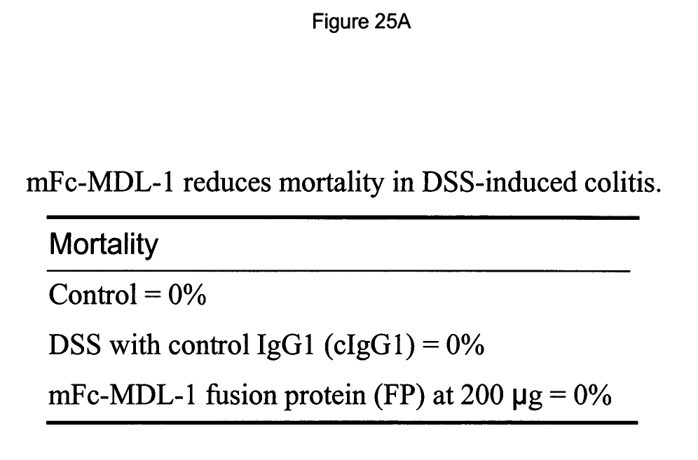

At present, the commonly used medicines for Crohn's disease are 5-aminosalicylic acid (5-ASA) and prednisolone. Our study showed that one dose of mFc-mMDL-1 fusion protein reduced disease activity at a level comparable to daily use of prednisolone yet the effect lasted for 7 days (FIGS. 24A and B).

B. DSS Colitis

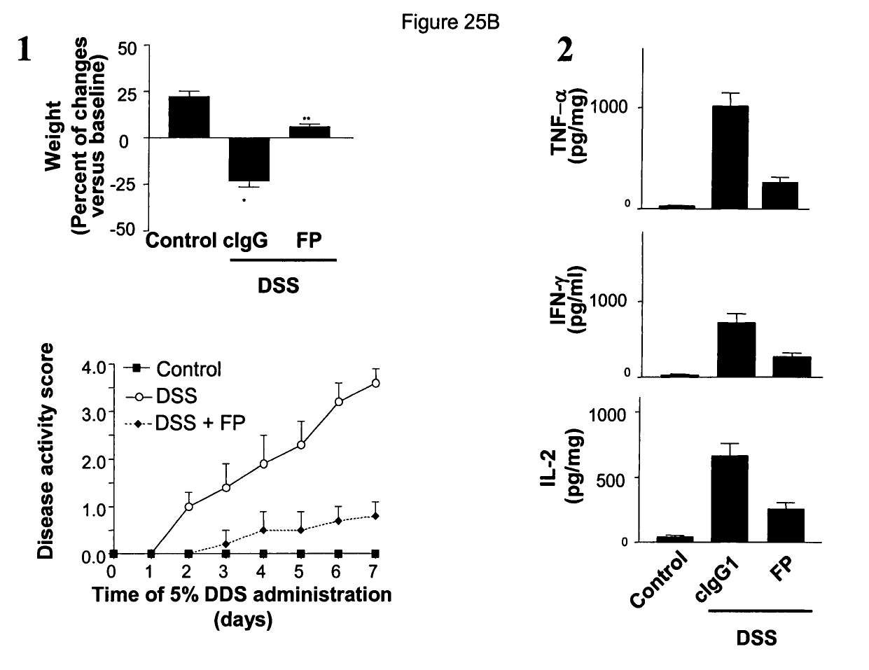

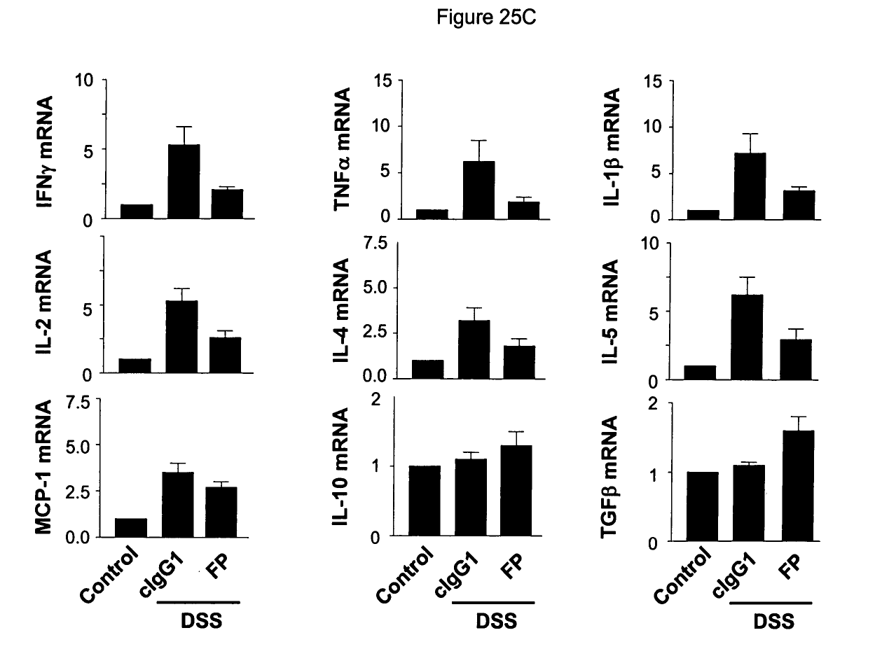

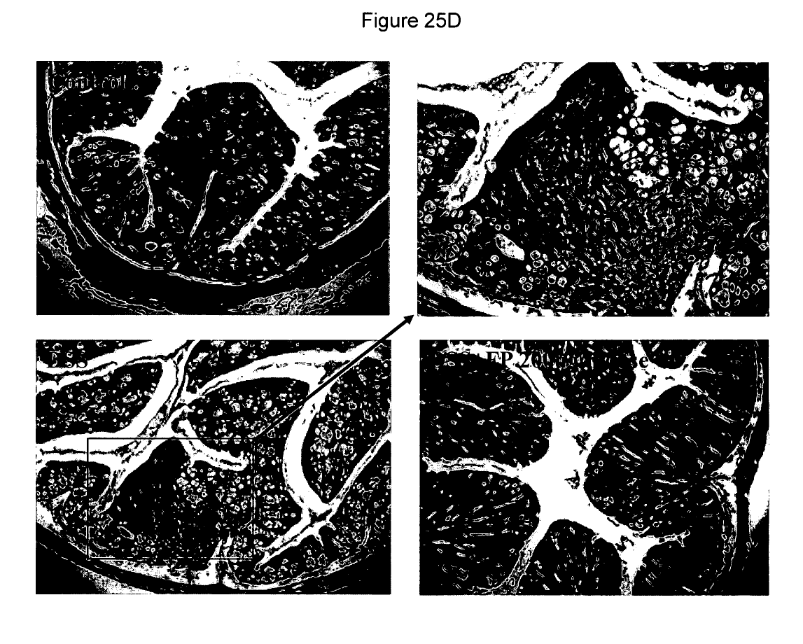

mFc-mMDL-1 was also efficacious in the DSS colitis model. After administration of 5% DSS, control IgG treated group showed remarkable weight loss. By comparison mFc-mMDL-1 fusion protein reversed the weight loss and reduced the level of pro-inflammatory mediators (FIGS. 25A-D).

### Example 13

### DAP12−/− and DAP12tg DSS-Colitis

As stated in section 2.2, in order to transduce signal, MDL-1 needs to interact with its adaptor molecule DAP12. Studies have shown that both DAP12 knock out and transgenic mice demonstrated immunological abnormalities.

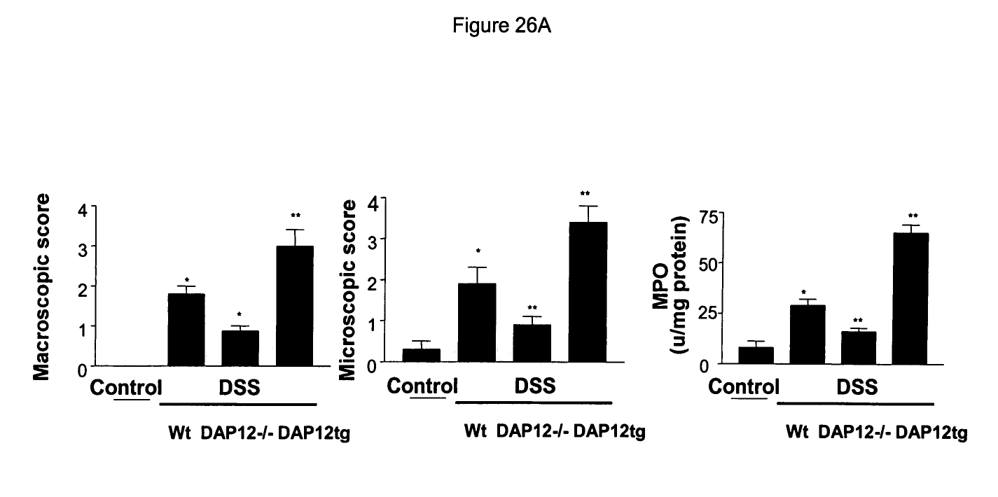

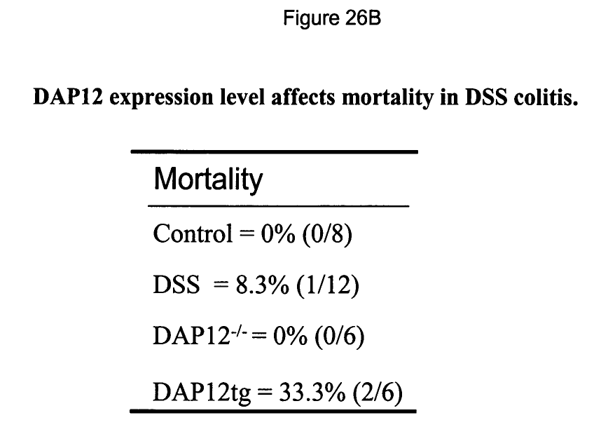

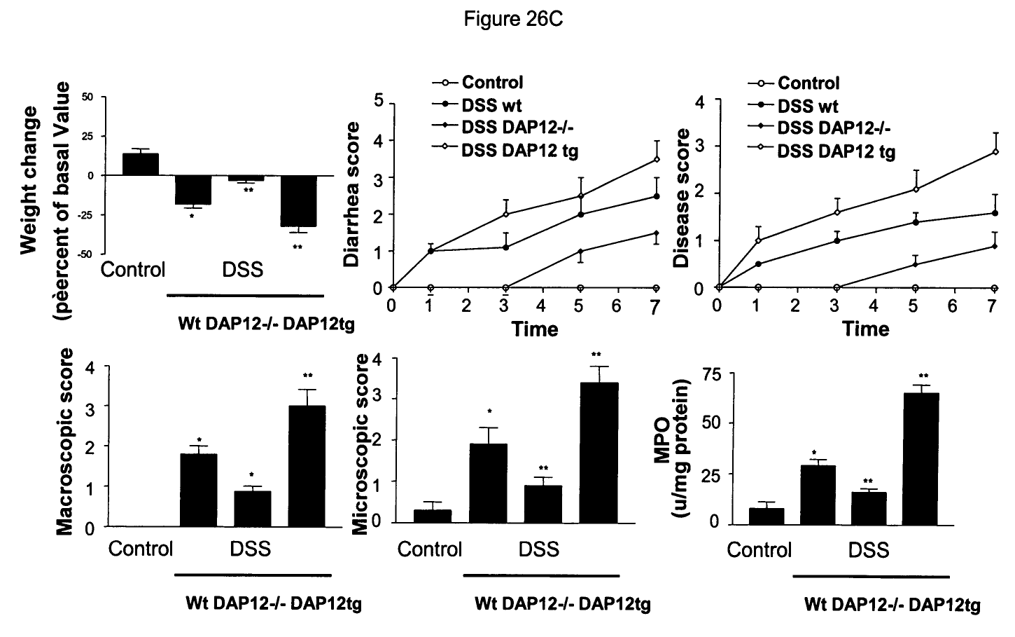

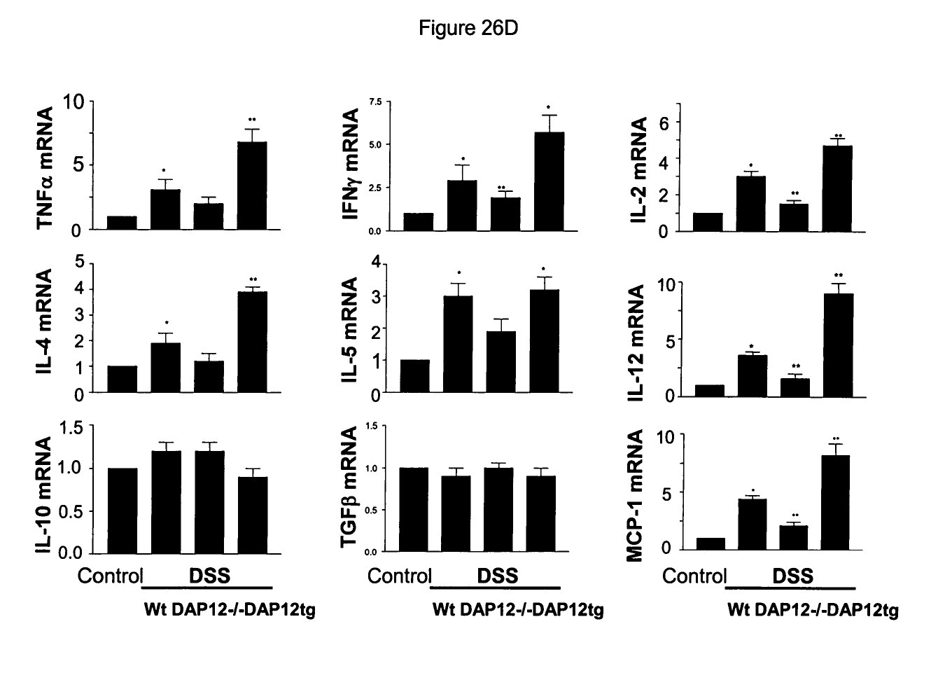

To test the role of DAP12 signaling pathway in colitis, both DAP12−/− and DAP12tg mice were used in DSS-induced colitis. Lack of DAP12 ameliorated the disease. On the contrary, high level expression of DAP12 exacerbated the disease (FIGS. 26A-D).

### Example 14

### TNBS Colitis—Therapeutic Treatment

So far, all efficacy data of mFc-mMDL-1 fusion protein were generated using prophylactic treatment regimen. To test whether MDL-1 fusion protein is capable of treating the disease after the establishment of colitis, we performed further efficacy studies in TNBS-colitis.

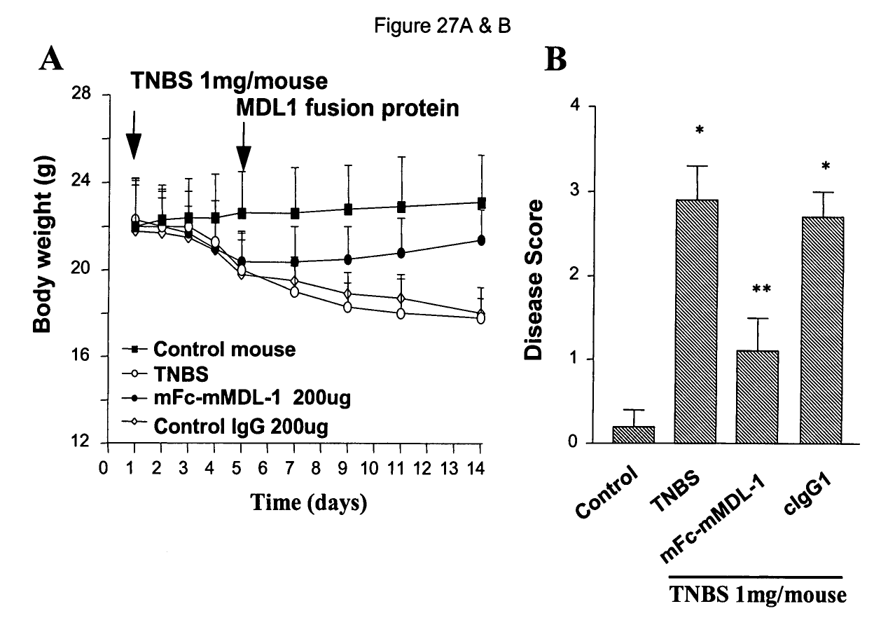

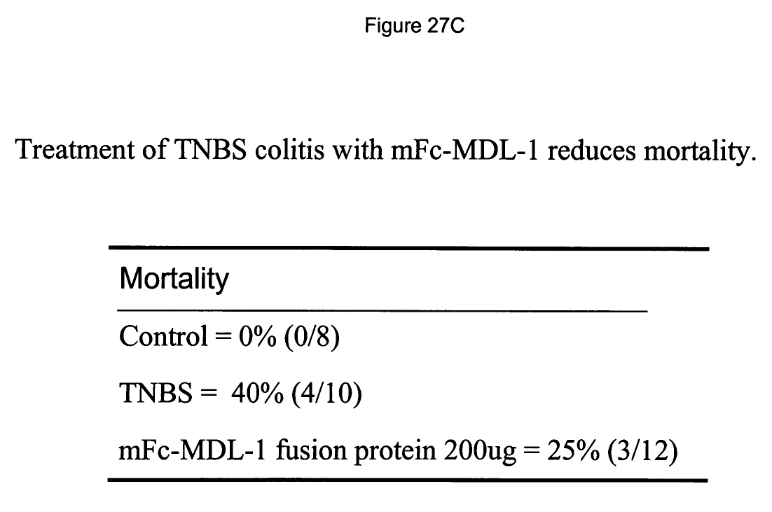

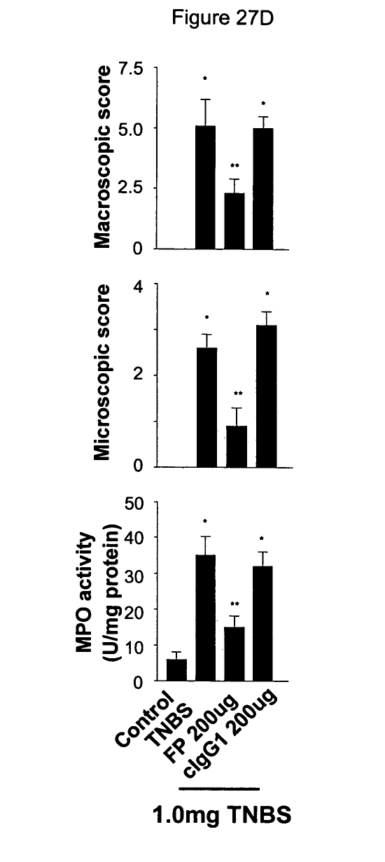

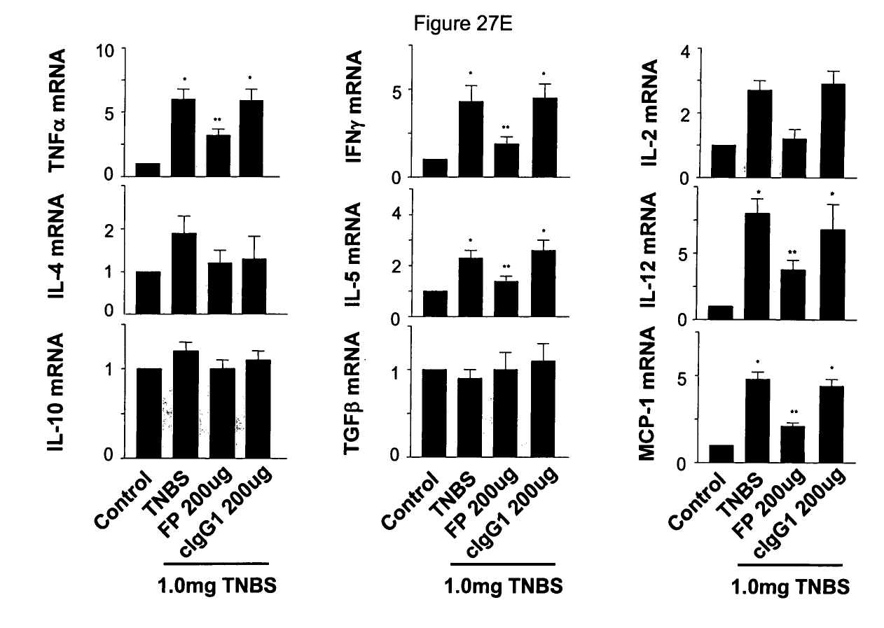

mFc-mMDL-1 fusion protein was administrated on day 5 after colitis has fully developed. After only one administration, mFc-mMDL-1 reversed the weight loss (FIG. 27A). At the end of study after 14 days, both disease score and level of pro-inflammatory mediators were markedly reduced in MDL-1 fusion protein treated group compared to control IgG treated group (FIGS. 27B-E).

### Example 15

### Evaluation of MDL-1 Decoy Fusion Protein in Mouse Chronic EAE

TREM-1 is a novel receptor of the IgG superfamily expressed on human neutrophils and monocytes, which promotes cell activation through the association of DAP12 (e.g., Bouchon et al. (2000). The ligand for TREM-1 is still unknown. Recently, it has been reported that mouse TREM-1-Fc decoy fusion protein can block inflammation in animal models and can prevent death caused by septic shock in animal models (e.g., Bouchon et al. (2001) J. Immunol. 164:4991-4995). The use of TREM-1-Fc decoy fusion protein in vivo validates this approach to evaluate MDL-1 as a therapeutic target for the treatment of MS. The mMDL-1-Fc fusion protein can be used in the mouse chronic EAE (mcEAE) model, an animal model of human MS that is induced by the adoptive transfer of PLP-specific immune cells in SJL mice. Since the present inventors have shown that MDL-1 expression correlates with the disease course in mcEAE, this model can be used to determine whether blocking the interaction between MDL-1 and its ligand ameliorates disease. Both human IgG1 and mMDL-1-Fc can be used in these studies. The effects of mMDL-1-Fc on both the clinical and pathohistological aspects of the disease can therefore be assessed. It is expected that the mMDL-1 decoy protein can decrease the incidence or ameliorate the clinical scores of paralysis of mcEAE.

For in vivo assays, it is important that the preparations are endotoxin-free. The endotoxin level can be tested before performing any in vivo assays using the Limulus Amebocyte Lysate assay (BioWhittaker). To further exclude the effects of endotoxin, fusion proteins can be heat-inactivated. Heat-inactivation will denature the fusion proteins and should have no activating effect in in vivo assays.

The present inventors have successfully expressed a fusion protein using mMDL-1 ECD linked to the Fc portion of human IgG1 in both baculovirus and mammalian systems. The mammalian expressed human IgG1 Fc fusion protein, mMDL-1-hFc, can be used for the EAE animal study. Pharmacokinetic studies can be performed to determine the profile of mMDL-1-hFc in vivo before the EAE animal study. The presence of mouse anti-human Fc antibody production can be monitored after administration of the mMDL-1-hFc fusion protein. If anti-human Fc antibody is present at high titer, the mMDL-1 fusion protein can be used with mouse IgG1 Fc.

### Example 16

### Generation of Biochemical and Cellular Tools to Identify the MDL-1 Ligand

The identification of a cognate ligand of MDL-1 can provide a direct means by which to inhibit its interaction with MDL-1. The present inventors have generated a cellular probe, CD3zH912Z1, and have validated it with the surrogate ligand α-hMDL1 antibody. Thus, one approach for identifying and isolating an MDL-1 ligand is, e.g., to expression clone the ligand with the CD3zH912Z1 probe by a functional screen of cDNA library expressed as pools in recipient cells.

Alternatively, known approaches for isolating and identifying a ligand can be used. For example, the ligands for murine NKG2D, another C-type lectin receptor that signals via the DAP10 adaptor protein (e.g., Diefenbach et al. (2000) Nature Immunol. 1:119-126; Cerwenka et al. (2000) Immunity 12:721-727; Bauer et al. (1999) Science 285:727-729). Cerwenka et al. (2000) Immunity 12:721-727) screened an expression library with a fusion protein consisting of the extracellular domain of NKG2D fused to the C-terminus of IgG constant domain. Similarly, Diefenbach et al. (2000) Nature Immunol. 1:119-126) generated tetramers that consisted of the ECD of NKG2D with a biotinylation site fused at the N-terminus. After enzymatic biotinylation, the tagged ECD protein was tetramerized with streptavidin conjugated to phycoerythrin (SA-PE). Both of these groups identified the ligands to NKG2D by expression of cDNA libraries in recipient cells followed by flow cytometry.

Further, DAP12 associated receptor (Ly49H) has been identified (e.g., Arase et al. (2001) Science 296:1323-1326). Molecular identification of the ligand for Ly49H was achieved by expression cloning from a retroviral library screened with a cellular probe expressing the green fluorescent protein (GFP) reporter gene upon ligation with the transfected Ly49H and DAP12 cDNAs.

Such biochemical tools can be used to screen expression libraries for the MDL-1 ligand. For example, two epitope tagged proteins, Fc tagged MDL-1-ECD and FLAG epitope tagged MDL-1-ECD can be generated. These protein tools cam then be used to screen retroviral libraries for the ligand to MDL-1 as well as to stain cells and tissues that express the MDL-1 ligand by FACS and IHC and to characterize the ligand once identified in the absence of ligand-specific antibodies. They provide useful controls for each other as they have molecularly distinct epitope tags. Additionally, a fusion protein with the FLAG epitope and a biotinylation site fused to the MDL-1-ECD can be generated and used for tetramerization. This MDL-1 tetramer can be used to screen retroviral libraries for the ligand to MDL-1 as well as to stain cells and tissues that express the MDL-1 ligand by FACS. The MDL-1 tetramer is a multivalent reagent that will presumably interact with the MDL-1 ligand with a high affinity as has been demonstrated for other tetramerized proteins (e.g., Altman et al. (1997) Science 274:94-96; Vance et al. (1999) J. Exp. Med. 190:1801-1812) and may offer an advantage in sensitivity of detection over other protein tools.

### Example 17

### Generation of cDNA Libraries to Screen for the MDL-1 Ligand

MDL-1 is expressed on activated macrophages that can interact with a variety of other cell types, all potentially expressing the MDL-1 ligand. Thus, primary cells isolated from human peripheral blood can be tested for the ability to stimulate the MDL-1 reporter cell CD3zH912Z1. Additionally, a panel of primary cells differentiated in vitro can be tested with CD3zH912Z1. The lineages to be tested in this panel of cells include macrophage; dendritic cells (DCs), Th1 and Th2 differentiated CD4 T cells and CD3 T cells activated under a variety of pro-inflammatory conditions. The present inventors also tested tumor cell lines of various origins, and cell lines of CNS origin. Any cells or cell lines that specifically express the MDL-1 ligand can be used to generate cDNA for library construction. The activation of the CD3zH912Z1 reporter cell can be correlated with surface expression by FACS with either the monomeric or tetrameric MDL-1 protein tools described herein. Libraries for expression cloning can be made e.g., from the cell source expressing the highest amount of MDL-1 ligand protein and the amount correlates with mRNA levels.

Further, mRNA can be isolated from various tissue of disease models, e.g., EAE spinal cords. The present inventors have demonstrated that MDL-1 mRNA levels increase relative to background at the onset of disease (d11), and MDL-1 mRNA levels reach a maximum at the peak of disease score (d17) to approximately 8-fold over background (FIG. 4). In one approach, where MDL-1 ligand expression correlates with MDL-1 expression, cDNA libraries from mRNA can be isolated at and around the peak of MDL-1 expression.

Different types of libraries can be made for expression cloning the MDL-1 ligand. For example, a plasmid library can be generated using a vector that contains a high expression promoter such as the cytomegalovirus promoter (pCMV) or the promoter for elongation factor (pEF) as well as the SV40 origin of replication for high level replication in recipient cells that express the large T antigen. Additionally, a retroviral library can be generated using a vector having a high expression viral promoter. The CD3zH912Z1 reporter cell or a murine equivalent can be used to screen the plasmid library in cDNA pools, while the protein reagents can be used to screen the retroviral library by FACS analysis.

### Example 18

### Expression Cloning the MDL-1 Ligand in Recipient Cells

The surrogate ligand, α-MDL-1 antibody provides a positive control for the functional assay. Further, the specificity of the protein reagents that measure binding of the soluble receptor in the form of the MDL-1 tetramer, Fc-MDL-1-ECD or FLAG-MDL-1-ECD protein, can be determined by competition between the soluble reagents for binding to potential ligand expressing cells. The plasmid libraries can be screened in pools of cDNA that are replicated in bacteria and isolated as minipreps. Similar methods known in the art can also be used (e.g., Mendoza et al. (1997) Immunity 7:461-472; Malarkannan et al. (1998) J. Immunol. 161:3501-3509; Malarkannan et al. (2000) Immunity 13(3):333-344; Mendoza et al. (2001) Methods Mol. Biol. 156:255-263). For example, cDNA pools can be transiently expressed in recipient cells and the recipient cells screened for expression of MDL-1 ligand by co-culture with the CD3zH912Z1 line by function. Potential positive pools can be cloned from cDNA master plates containing the library pools by repeated rounds of the transfection and functional assay until the cDNA is cloned (e.g., Mendoza et al. (2001) Methods Mol. Biol. 156:255-263).

Also for example, the retroviral libraries can be screened by transduction of the library into recipient cells upon which the viral chromosome can either integrate into the host genome or replicate episomally. Recipient cells can be screened e.g., either with the MDL-1 tetramer, FLAG-MDL-1-ECD or Fc-MDL-1-ECD by flow cytometry. Cells that bind these reagents can then be sorted to enrich for the cell expressing the putative MDL-1 ligand until the cells are clonal or alternatively, plasmid DNA can be isolated from transfected cells, amplified and recipient cells re-transfected until the cells are clonal. In order to recover the DNA encoding the MDL-1 ligand, retroviral vector primers can be used to amplify the cDNA by polymerase chain reaction (PCR) from cellular genomic DNA or plasmid DNA can be cloned.

### Example 19

### Demonstration of Disease Association by Studying Expression of MDL-1 and Its Ligand in Disease Models

Myeloid DAP12-associating lectin, MDL-1, is a surface protein expressed primarily in myeloid cells. A direct interaction between MDL-1 and the adaptor protein DAP12 is reportedly necessary for its signaling function. The data provided herein demonstrate a direct correlation between an increase in MDL-1 expression and clinical disease score in a murine EAE model of MS. Furthermore, the data demonstrate that activation of MDL-1 with specific antibodies generated against MDL-1 triggers the release of a variety of pro-inflammatory chemokines and cytokines from macrophages and strongly implicates MDL-1 in inflammatory processes.

For example, MDL-1 expression is found in brain pons and choroid plexus from a patient with Hungtington's disease, and brain hippocampus from a patient with multiple microinfarcts (Incyte database). Further, the present inventors have shown that MDL-1 expression correlates with disease progression in the mcEAE model. Additionally, the present inventors have generated and characterized anti-MDL-1 antibodies and MDL-1 fusion proteins for FACS and IHC analysis. As described herein, MDL-1 expression can be studied by IHC and FACS in both the CNS and the periphery to further define the role of MDL-1 in inflammatory disease pathology, e.g., in MS, IBD, and arthritis. For example, the expression of the MDL-1 ligand can be tested by IHC and FACS in EAE and MS tissues. Such studies are important to provide further information on the role of MDL-1 and its ligand in such inflammatory diseases and as therapeutic targets for treatment of such MDL-1 mediated inflammatory diseases.

For example, inflammatory bowel disease (IBD) is characterized by chronic and relapsing bowel inflammation. Crohn's disease (CD) and ulcerative colitis are the two main forms of IBD. There are ˜1-2 million IBD patients in the USA, with ˜ half having CD. CD is associated with changes in the immune system, including massive mucosal infiltration of neutrophils, lymphocytes, and macrophages. The production by these infiltrating cells of pro-inflammatory chemokines and cytokines, such as IL-2, IFN-γ, and TNF-α, plays a pivotal role in CD pathogenesis. Anti-TNF therapies are used to treat subsets of patients with Crohn's disease, but there is high medical need for innovative drugs with a superior side effect profile to anti-TNF therapies and to treat patients not responding to anti-TNF therapy.

Inflammatory bowel diseases (IBD), which are comprised of Crohn's disease and ulcerative colitis, are characterized, e.g., by the clinical course of succession of relapses and remissions, and by chronically relapsing inflammation of the bowel. Current statistics indicate that there are 1-2 million Americans suffering from IBD with half of them diagnosed as having Crohn's disease (e.g., Head and Jurenka (2004) Alternative Med. Rev. 9:360-401). IBD causes much personal suffering and disablement for patients and represent a substantial economic burden on healthcare resources.

Reportedly, genetic susceptibility, environmental triggers, and immune activation are three major contributory factors to the pathogenesis of inflammatory diseases such as IBD. Immune abnormality in inflammatory diseases such as IBD is associated with several changes in the immune system, including massive infiltration of neutrophils, lymphocytes, and macrophages (e.g., Wen and Fiocchi (2004) Clin. Dev. Immunol. 11:195-204). It has been demonstrated that the production of pro-inflammatory chemokines and cytokines, such as MCP-1, IL-2, IFN-g, and TNF-a, by infiltrating inflammatory cells plays a pivotal role in the pathogenesis of inflammatory diseases, including e.g., IBD. Anti-TNF-a monoclonal antibody, Infliximab, has been proved to achieve clinical improvement and induce remission in patients with moderate-to-severe luminal and fistular Crohn's disease refractory to other treatments (e.g., Sandborn (2005) Rev. Gastroenterol. Disord. 5:10-18).

Taken together, the findings of the present inventors as described herein, indicate that MDL-1 may play a role in the pathogenesis of inflammatory diseases, and suggest a novel therapeutic target for treatment of such inflammatory diseases. Based on these findings, the present invention provides therapeutic compositions and methods for the treatment of inflammatory diseases, e.g., MS, IBD, and arthritis. In some embodiments, the compositions and methods of the present invention modulate MDL-1 activity. In particular, in one embodiment, the compositions and methods of the present invention inhibit the activity of MDL-1 by blocking the receptor on myeloid cells.

Thus, as demonstrated herein by the present inventors, the role of MDL-1 in macrophage-mediated inflammation and its association with CD suggests that MDL-1 is an attractive and innovative therapeutic target for the treatment of inflammatory diseases particularly those mediated by MDL-1. Examples of such diseases include, but are not limited to, IBD, MS, and arthritis.

### SUMMARY

The present inventors have generated α-mouse MDL-1 (α-mMDL-1) antibodies which could function as a surrogate ligand for the mMDL-1 receptor in functional assays. In addition, α-mMDL-1 antibodies can also be used to characterize the biological activity of mMDL-1-Fc decoy protein in vitro.

The present inventors have established three functional assay systems for the identification and characterization of α-human MDL-1 antibodies and hMDL-1-Fc fusion proteins. Based on the results of the human MDL-1 studies, similar approaches can be utilized for the screening and characterization of α-mMDL-1 antibodies and mMDL-1-Fc fusion proteins.

RBL-2H3 and BWZ.36 cells can be cotransfected with mMDL-1 (or mMDL-1CD3ζ chimera) and mDAP12 (Flag-tagged in the N-terminus) expression vectors. Stable transfectants selected with antibiotics and identified using α-mMDL-1 and α-Flag antibodies by FACS analysis for the expression of both mMDL-1 and mDAP12. RBL- or BWZ-mMDL-1/mDAP12 stable transfectants can be used for serotonin release or β-galactosidase report assays. The present inventors previously demonstrated that α-Flag mAb can bind and cross-link extracellular domain of hDAP12 to release serotonin in RBL-hMDL-1/hDAP12 transfectants (data not shown). To validate serotonin release or β-galactosidase activation before antibody testing, both RBL- and BWZ-mMDL-1/mDAP12 stable transfectants can first be cross-linked with α-Flag mAb. α-mMDL-1 antibodies will then be tested using functional assays in characterized transfectants.

Antibodies that are capable of inducing serotonin release or β-galactosidase activation in RBL- or BWZ-mMDL-1/mDAP12 transfectants, can then function as surrogate ligands to activate murine macrophages which express MDL-1. FACS analysis can be carried out to determine surface expression of MDL-1 on either bone marrow derived or thioglycollate-elicited peritoneal macrophages. After confirmation of MDL-1 expression on ex vivo macrophages, further experiments can be performed to determine the profile of chemokines and cytokines released from α-mMDL-1 antibody activated macrophages. Further, mouse macrophage-like cell lines such as J774A.1 and RAW264.7 can be used for chemokine and cytokine release studies. Experiments performed by the present inventors demonstrate that both J774A.1 and RAW264.7 cells express MDL-1 on their cell surfaces in FACS analysis (data not shown).

The present inventors have also shown that rabbits immunized with human MDL-1-ECD developed useful antibodies including activating polyclonal antibodies. Consequently, activating α-mMDL-1 antibodies can be developed for functional assays.

As demonstrated herein by the present inventors: one dose of mFc-mMDL-1 fusion protein is efficacious in both TNBS- and DSS-induced mouse colitis models; the lack of DAP12 ameliorates the disease, and over-expression of DAP12 exacerbates the disease; MDL-1 fusion protein is effective in both prophylactic and therapeutic treatment regimens; and the action of MDL-1 fusion protein may be mediated by reducing the production of pro-inflammatory chemokines and cytokines.

Both pharmacological and genetic approaches demonstrated by the present inventors established that MDL-1 and its signal-transducing adaptor protein DAP-12 contribute to the pathology of two experimental colitis models with distinct etiologies (DSS and TNBS colitis). For example:
DAP-12^{-/-} mice are protected from colitis whereas overexpressing DAP-12 transgenic mice have exacerbated disease.
A mouse MDL-1 fusion decoy protein (mFc-mMDL-1) ameliorates disease.
mFc-mMDL-1 efficacy correlates to decreased mucosal TNF-α, MCP-1 and IFN-γ.
mFc-mMDL-1 prevents colitis onset and treats established colitis and due to a robust PK profile provides efficacy when given as a single dose in 7 to 14 day studies.

Further, therapeutic use in humans, a fully human fusion protein consisting of human MDL-1 extracellular domain and the hinge, CH2, and CH3 of human IgG1 that can be generated and expressed at high level in CHO cells in a system suitable for GMP/GLP production. Further, a fusion protein can be generated having a PK profile suitable for once-weekly or less frequent dosing.

Materials and Methods

Human colon samples. Surgical colon specimens from 8 patients were used for RT-PCR analysis. Five patients presented only a colon localization of Crohn's disease and 3 patients also presented intestinal involvement. All patients presented a disease flare at the moment of the resection (mean Crohn's disease activity index was 427.0±33.2) that had not responded to corticosteroid/immunosuppressive therapy. Control colons were obtained from macroscopically normal colons removed from age and sex matched patients undergoing total or partial colectomy for colon cancer.

Cells. HEK293 cells expressing Epstein-Bar Virus (EBV) EBNA-1 protein (Invitrogen) were cultured in DMEM (Gibco-BRL Life Technologies) supplemented with 8% fetal bovine serum, 2 mM L-glutamine and 0.3 mg/ml G 418 in monolayers. The cells were adapted to suspension culture using 293 SFM II (Gibco-BRL Life Technologies) containing 4 mM L-glutamine, 10% FBS, penicillin/streptomycin reagent and 0.05 mg/ml G 418 prior to transfection. Cells were maintained at 37° C. in humidified incubators with 5% CO₂ and 95% air.

MDL-1 fusion protein. The mouse MDL-1 fusion protein was created by fusing an extracellular domain of mouse MDL-1 (amino acids 28 to 190) to the C-terminus of the hinge, CH2 domain, and CH3 domain of mouse IgG1 (amino acids 98 to 324) having a two amino acid linker (amino acid AS). The expression construct was expressed transiently in HEK293 cells, and fusion protein was purified on an anti-mouse IgG1 Protein G affinity column. The protein concentration was determined by ELISA assay. The endotoxin level, as determined by the Limulus Amebocyte Lysate Assay (BioWhittaker), was <0.9 EU/mg of mFc-mMDL-1 fusion protein. mFc-mMDL-1 fusion protein was further characterized by SDS-PAGE, N-terminal sequencing, and immunoreactivity analysis (Western blot and ELISA) (unpublished data).

Antibodies. The following monoclonal antibodies were from Cell Signaling: Anti Erk 1,2 and anti-phospho Erk1,2. Polyclonal rabbit antibodies specific for MDL-1 were raised against baculovirus expressed extracellular domain of human or mouse MDL-1. Polyclonal rabbit antibodies specific for DAP12 were raised with a peptide spanning amino acids 79-113 of human DAP12 coupled to keyhole limpet hemocyanin. Rabbit antisera were purified by using Protein A agarose beads (Roche Diagnostics).

Isolation of human CD14+ cells from PMBC. Human PBMCs were obtained from normal individual donors and isolated by density gradient centrifugation through a Ficoll-Hypaque gradient (Pharmacia Biotech AB, Uppsala, Sweden). Monocytes, T and B lymphocytes, NK cells and granulocytes were isolated by negative selection using magnetic cell sorting according to the manufacturer instructions (Mylteni Biotec, Milan, Italy). CD14+monocytes were purified from PBMC by magnetic cell sorting using CD14 MicroBeads (Miltenyi Biotec). Monocytes were differentiated in vitro for 7 days in serum free medium X-VIVO 50 (BioWhittaker) containing 25 ng/ml recombinant human GM-CSF (rhGM-CSF) (R&D Systems). Culture medium with rhGM-CSF was changed at day 3. Differentiated macrophages were stimulated with anti-human MDL-1 or pre-immune antibody for 4 hr. Supernatants were collected and tested for production of TNF-α, IL-6, MCP-1, and MIP-1a by ELISA (R&D Systems).

Immunoprecipitation and immunoblotting. For co-immunoprecipitation experiments, macrophages were lysed in lysis buffer containing 1% Digitonin, 10 mM Tris [pH7.5], 150 mM NaCl, 1 mM NaF, 1 mM sodium orthovanadate, 1 mM PMSF, and Protease inhibitor cocktail (Roche Diagnostics). Insoluble fraction was removed by centrifugation. Lysates were then immunoprecipitated with anti-human MDL-1 polyclonal antibodies and Protein A agarose beads (Roche Diagnostics). After washing in PBS, precipitates were fractionated by SDS-PAGE and immunoblotted with anti-DAP12 antibodies. For immunoblot analysis, total cell lysates were fractionated by standard SDS-PAGE (4%-20% Tris-Glycine, Precast gel, Cambrex Bio Science Rockland), and the resolved proteins were transferred electrophoretically to nitrocellulose membrane. The membranes were then probed with antibodies. Horseradish peroxidase (HRP) conjugated secondary antibodies (Jackson ImmunoResearch Laboratories) incubated membranes were developed by the enhanced chemiluminescence (ECL) detection reagents (Amersham Biosciences).

Animals. Six- to eight-week-old Balb/c female mice, RAG-1^{-/-} mice were obtained from Charles River (Monza, Italy). DAP12^{-/-} and DAP12 trangenic mice on a C57BL/6 background were generated as described elsewhere^{(24,26)}. Syk deficient mice were kindly donated by Victor L. J. Tybulewicz, Division of Immune Cell Biology National Institute for Medical Research, London U.K.⁽²⁷⁾. The study protocol was approved by the Animal Study Committees of the University of Perugia according to governmental guidelines for animal care.

Colitis models and study design. For induction of TNBS mice were administered TNBS (Sigma Chemical Co, St Louis, Mo.) dissolved in 50% ethanol via intrarectal catheter as described previously^{(23,31-33)}. Control mice received 50% ethanol alone. For induction of DSS colitis, BALB/c mice were fed 5% (wt/vol) DSS (molecular weight, 40 kDa; ICN Biomedicals Inc.,) dissolved in filtered water for 7 days. In both models animals were monitored daily for appearance of diarrhea, loss of body weight, and survival. At the end of the experiment, surviving mice were sacrificed, blood samples collected by cardiac puncture, and a 5 cm segment of colon was excised, weighed, and evaluated for macroscopic and microscopic damage and cytokine content.

To assess whether MDL-1 blockage could protect against development of colitis, BALB/c mice receiving 1.5 mg TNBS were randomized and received either no treatment (i.e. TNBS alone), or TNBS plus αMDL-1 fusion protein at the dose of 50, 100 or 200 μg/kg. The αMDL-1 fusion protein was administered intraperitoneally (i.p.) on day 1 and mice were sacrificed at day 7. Control mice were treated with the control IgG (200 μg/kg). Lastly, to address whether anti MDL-1 treatment was beneficial in treating established colitis, administration of the αMDL-1 fusion protein was started 7 days after colitis induction (1.5 mg TNBS). Balb/c mice were treated i.p. on day 7 with a single dose of 200 μ/kg of either αMDL-1 fusion protein or control IgG. At day 15, mice were sacrificed and their colons analysed.

To investigate the role of DAP12 and Syk in intestinal inflammation, DAP12 deficient and DAP12 transgenic mice as well as Syk deficient mice with TNBS (data not shown) or DSS (see above) and the αMDL-1 fusion protein or the control IgG (200 μg/kg) were administered on day 1 (see above). To investigate the role of monocytes and T cells on protection exerted by αMDL-1 fusion protein on DSS-induced colitis, RAG-1^{-/-} and a BALB/c mice were treated by intracolonic injection of TNBS (1.5 mg/mouse), alone or in combination with the αMDL-1 fusion protein or the control IgG (200 μg/kg i.p.) and sacrificed after 7 days.

Macroscopic and histologic grading of colitis. Colons were examined under a dissecting microscope (×5) and graded for macroscopic lesions on a scale from 0 to 10 based on criteria reflecting inflammation, such as hyperemia, thickening of the bowel, and the extent of ulceration. For histologic examination, a colon specimen located precisely 2 cm above the anal canal was obtained, fixed in 10% buffered formalin phosphate, embedded in paraffin, sectioned, and stained with hematoxylin and eosin (H&E). Inflammation on microscopic cross-sections was graded semi-quantitatively from 0 to 4 (0: no signs of inflammation; 1: very low level of inflammation; 2: low level of leukocyte infiltration; 3: high level of leukocyte infiltration, high vascular density, thickening of the colon wall; 4: transmural infiltrations, loss of goblet cells, high vascular density, thickening of the colon wall). Plasma and mucosal IL-1β, IL-2, IL-6, IFN-γ, TNF-α and MCP-1 concentrations were determined by ELISA (Endogen, Woburn, Mass.).

Isolation of LPMC. LPMC were isolated from freshly obtained colonic specimens as described previously. In brief, after excision of all visible lymphoid follicles, colons were digested with type IV collagenase (Sigma) for 20 min in a shaking incubator at 37° C.; this step was repeated twice. The released cells were then layered on a 40%-100% Percoll gradient (Pharmacia, Upsala, Sweden) and spun at 1,800 rpm to obtain the lymphocyte-enriched populations at the 40-100% interface. Gr-1⁻/CD14⁺ and CD4⁺ cells were purified from LPMC via negative selection using a macrophages and T cell isolation kits (Miltenyi Biotec). To measure cytokine production, 106 LPMC were placed for 48 hr onto uncoated culture wells. Culture supernatants were harvested and assayed for cytokine concentration by ELISA (Endogen).

Quantitative RT-PCR. Total RNA was isolated using TRIzol reagent (Life Technologies). RT-PCR primers were designed using the PRIMER3-OUTPUT software using published sequence data from the NCBI database. One μg of purified RNA was treated with DNaseI for 15 min at room temperature followed by incubation at 95° C. for 5 min in the presence of 2.5 mM EDTA. The RNA was reverse-transcribed with Superscript III (Invitrogen) in 20 μl reaction volume using random primers. For RT-PCR, 100 ng template was used in a 25 μl containing 0.3 μM of each primer and 12.5 μl of 2×SYBR Green PCR Master mix (Bio-Rad). All reactions were performed in triplicate and the thermal cycling conditions were as follows: 2 min at 95° C., followed by 50 cycles of 95° C. for 10 sec, and 60° C. for 30 sec in an iCycler iQ instrument (Biorad, Hercules, Calif.). The mean value of the replicates for each sample was calculated and expressed as the cycle threshold (CT: cycle number at which each PCR reaction reaches a predetermined fluorescence threshold, set within the linear range of all reactions). The amount of gene expression was then calculated as the difference (CT) between the CT value of the sample for the target gene and the mean CT value of that sample for the endogenous control. Relative expression was calculated as the difference (CT) between the CT values of the test control sample (WT) for each target gene. The relative expression level was expressed as 2-CT

Statistical analysis. All values in the Figs. and text are expressed as mean±SE. The variation between data sets was tested with ANOVA, and the significance was tested with unpaired t-tests, with a Bonferroni modification for multicomparison of data. Differences were considered significant when P was <0.05.

General Methods

Standard methods are described or referenced, e.g., in Maniatis, et al. (1982) Molecular Cloning, A Laboratory Manual, Cold Spring Harbor Laboratory, Cold Spring Harbor Press; Sambrook, et al. (1989) Molecular Cloning: A Laboratory Manual, (2d ed.), vols. 1-3, CSH Press, NY; Ausubel, et al., Biology, Greene Publishing Associates, Brooklyn, N.Y.; or Ausubel, et al. (1987 and Supplements) Current Protocols in Molecular Biology, Greene/Wiley, New York. Methods for protein purification include such methods as ammonium sulfate precipitation, column chromatography, electrophoresis, centrifugation, crystallization, and others. See, e.g., Ausubel, et al. (1987 and periodic supplements); Deutscher (1990) “Guide to Protein Purification” in Methods in Enzymology, vol. 182, and other volumes in this series; and manufacturer's literature on use of protein purification products, e.g., Pharmacia, Piscataway, N.J., or Bio-Rad, Richmond, Calif. Combination with recombinant techniques allow fusion to appropriate segments, e.g., to a FLAG sequence or an equivalent which can be fused via a protease-removable sequence. See, e.g., Hochuli (1989) Chemische Industrie 12:69-70; Hochuli (1990) “Purification of Recombinant Proteins with Metal Chelate Absorbent” in Setlow (ed.) Genetic Engineering, Principle and Methods 12:87-98, Plenum Press, N.Y.; and Crowe, et al. (1992) OIAexpress: The High Level Expression & Protein Purification System QUIAGEN, Inc., Chatsworth, Calif.

Standard immunological techniques are described, e.g., in Hertzenberg, et al. (eds. 1996) Weir's Handbook of Experimental Immunology vols. 1-4, Blackwell Science; Coligan (1991) Current Protocols in Immunology Wiley/Greene, New York; and Methods in Enzymology volumes. 70, 73, 74, 84, 92, 93, 108, 116, 121, 132, 150, 162, and 163. Assays for neural cell biological activities are described, e.g., in Wouterlood (ed. 1995) Neuroscience Protocols modules 10, Elsevier; Methods in Neurosciences Academic Press; and Neuromethods Humana Press, Totowa, N.J. Methodology of developmental systems is described, e.g., in Meisami (ed.) Handbook of Human Growth and Developmental Biology CRC Press; and Chrispeels (ed.) Molecular Techniques and Approaches in Developmental Biology Interscience.

FACS analyses are described in Melamed, et al. (1990) Flow Cytometry and Sortina Wiley-Liss, Inc., New York, N.Y.; Shapiro (1988) Practical Flow Cytometry Liss, New York, N.Y.; and Robinson, et al. (1993) Handbook of Flow Cytometry Methods Wiley-Liss, New York, N.Y.

Computer sequence analysis is performed, e.g., using available software programs, including those from the GCG (U. Wisconsin) and GenBank sources. Public sequence databases were also used, e.g., from GenBank and others.

We claim:

1. An MDL-1 fusion protein that modulates an MDL-1 activity in a mammalian cell, in vivo or in vitro.

2. The MDL-1 fusion protein of claim 1, wherein said MDL-1 activity is the binding of MDL-1 to a cognate ligand.

3. The MDL-1 fusion protein of claim 1, wherein said MDL-1 activity is the modulation of a DAP-12 activity.

4. The MDL-1 fusion protein of claim 1, wherein said fusion protein decreases said MDL-1 activity.

5. The MDL-1 fusion protein of claim 4, wherein said decrease in MDL-1 activity results in a decrease in DAP-12 activity.

6. The MDL-1 fusion protein of claim 1, wherein said fusion protein increases said MDL-1 activity.

7. The MDL-1 fusion protein of claim 1, wherein said fusion protein comprises an amino acid sequence encoding an Fc fragment and an amino acid sequence encoding an extracellular domain of MDL-1.

8. The MDL-1 fusion protein of claim 7, wherein said amino acid sequence encoding an Fc fragment is SEQ ID NO: 7.

9. The MDL-1 fusion protein of claim 7, wherein said amino acid sequence encoding an extracellular domain of MDL-1 is SEQ ID NO: 11.

10. The MDL-1 fusion protein of claim 7, wherein said amino acid sequence is SEQ ID NO: 1 or SEQ ID NO: 3.

11. The MDL-1 fusion protein of claim 7, wherein said amino acid sequence encoding an Fc fragment is SEQ ID NO: 9.

12. The MDL-1 fusion protein of claim 7, wherein said amino acid sequence encoding an extracellular domain of MDL-1 is SEQ ID NO: 13.

13. The MDL-1 fusion protein of claim 7, wherein said amino acid sequence is SEQ ID NO: 5.

14. The MDL-1 fusion protein of claim 1, wherein said MDL-1 fusion protein binds to an MDL-1 cognate ligand.

15. The MDL-1 fusion protein of claim 14, wherein said MDL-1 fusion protein binds to the MDL-1 binding site of said cognate ligand.

16. The MDL-1 fusion protein of claim 1, wherein said mammalian cell is a human cell.

17. The MDL-1 fusion protein of claim 1, wherein said modulation of an MDL-1 activity is in vivo.

18. The MDL-1 fusion protein of claim 17, wherein said modulation of an MDL-1 activity is in a mammal.

19. The MDL-1 fusion protein of claim 18, wherein said mammal is a human.

20. A method for treatment of a disease in a patient, using the MDL-1 fusion protein of any one of claims 1-19, wherein said disease comprises an inflammatory process.

21. The method of claim 20, wherein said disease is inflammatory bowel disease (IBD).

22. The method of claim 21, wherein said patient is a human.

23. A method for treatment of an MDL-1 mediated disease in a patient, using the MDL-1 fusion protein of any one of claims 1-19.

24. The method of claim 23, wherein said MDL-1 mediated disease is inflammatory bowel disease (IBD).

25. The method of claim 24, wherein said patient is a human.

26. A method of diagnosing or monitoring treatment of an MDL-1 mediated disease in a patient, using the MDL-1 fusion protein of any one of claims 1-19.

27. The method of claim 26, wherein said MDL-1 mediated disease is inflammatory bowel disease (IBD).

28. The method of claim 27, wherein said patient is a human.

29. The use of the MDL-1 fusion protein of any one of claims 1-19, in the manufacture of a medicament for the treatment of a disease, wherein the disease comprises an inflammatory process.

30. The use of the MDL-1 fusion protein of any one of claims 1-19, in the manufacture of a medicament for the treatment of inflammatory bowel disease IBD.

31. A diagnostic or therapeutic imaging agent comprising the MDL-1 fusion protein of any one of claims 1-19.

32. A kit for diagnosing or monitoring treatment of an MDL-1 mediated disease in a patient, wherein said kit comprises the MDL-1 fusion protein of any one of claims 1-19.

33. The kit of claim 32, wherein said MDL-1 mediated disease is inflammatory bowel disease (IBD).

34. The kit of claim 33, wherein said patient is a human.

35. An expression vector encoding the MDL-1 fusion protein of any one of claims 1-19.

36. The expression vector of claim 35, wherein the sequence of said expression vector is SEQ ID NO: 19.

37. The expression vector of claim 35, wherein the sequence of said expression vector is SEQ ID NO: 20.

38. A cell comprising the expression vector of any one of claims 35, 36, and 37, wherein said MDL-1 fusion protein is expressed in said cell.

39. The cell of claim 38, wherein said cell is a mammalian cell.

40. The cell of claim 39, wherein said cell is a Chinese Hamster Ovary (CHO) cell.
